(19) United States
(12) Patent Application Publication
(10) Pub. No.: US 2016/0008376 A1
(43) Pub. Date: Jan. 14, 2016

(54) Compositions, Methods and Uses for Treating Gender-Biased Immune Disorders

(71) Applicant: Biocopea Limited, London (GB)

(72) Inventors: Gregory A. Stoloff, London (GB); John Brew, Hertfordshire (GB); Robin M. Bannister, Essex (GB); Olga Pleguezuelos Mateo, Bicester (GB); Wilson Caparrós-Wanderley, Buckinghamshire (GB)

(73) Assignee: Biocopea Limited, London (GB)

(21) Appl. No.: 14/796,582

(22) Filed: Jul. 10, 2015

Related U.S. Application Data
(60) Provisional application No. 62/023,082, filed on Jul. 10, 2014.

(51) Int. Cl. A61K 31/57; A61K 31/4196

(52) U.S. Cl. CPC A61K 31/57; A61K 31/4196

(57) ABSTRACT

The present specification discloses compositions comprising at least one therapeutic compound capable of modulating a level and/or activity of a hormone and methods and uses for treating a gender-biased immune disorder using such compositions.

This patent application claims the right of priority pursuant to 35 U.S.C. §119(e) and is entitled to the benefit of the filing date of U.S. Provisional Patent Application 62/023,082, filed on Jul. 10, 2014, the content of which is hereby expressly incorporated by reference in its entirety.

The major histocompatibility complex (MHC) protein family, also referred to as human leukocyte antigen (HLA), are a group of cell-surface glycoproteins that act as support structures for the presentation of endogenous and exogenous antigens to T and B lymphocytes for recognition and response. These proteins play a central role in enabling the immune system to differentiate between self from non-self (foreign) molecules, cells, and other substances. As such, these cell-surface histocompatibility antigens are the basis for protective immunity against pathogenic infections and disease and are the principal determinants of tissue type and transplant compatibility.

The three major classes of MHC proteins are the classical, highly polymorphic class I (MHC-I) and class II (MHC-II) glycoproteins and Class III MHC glycoproteins of the complement system (i.e. C2, C4a, C4b, Bf) that help to activate and maintain the inflammatory process of an immune response.

MHC-I glycoproteins are present on the surface of almost all nucleated cells (except red blood cells) and are important for displaying endogenous non-self antigens, that is, antigens produced inside the cell in order to elicit cellular immunity. Cells typically contain aberrant cytosolic protein, carbohydrates, polynucleotides, or other molecules like toxins derived either from protein turnover, defective replication, transcription or translation, or abnormal metabolism or catabolism, or during viral infection, intracellular microorganism infection, or cancerous transformation. Such cytosolic polypeptide fragments are degraded in the proteosome and peptides typically between 8 and 11 amino acids in length are bound to the cleft of MHC class I glycoproteins and displayed as epitopes on the cell surface to CD8⁺ cytotoxic T cells. Cytotoxic T cells (also known as TC, killer T cell, or cytotoxic T-lymphocyte (CTL)) are a population of T cells expressing CD8 receptors, in addition to T cell receptors (TCRs) and are specialized for inducing the death of other cells. When a CD8⁺ cytotoxic T cells CD8 receptor docks to a MHC class I molecule, if the CTL's TCR fits the epitope within the MHC class I molecule, the CTL triggers the cell to undergo programmed cell death by apoptosis. Thus, MHC class I helps mediate cellular immunity, the primary means to mediate the destruction of infected or malignant host cells. MHC-I presentation also determines compatibility of donors for organ transplant as well as one's susceptibility to an autoimmune disease via cross-reacting immunization.

MHC-II glycoproteins are expressed mainly by antigen-presenting cells (APCs), mostly dendritic cells, macrophages, B cells, and thymic epithelial cells, but are also expressed on certain normal cells and by cells under certain extreme situations. MHC-II glycoproteins are important for displaying exogenous non-self antigens, that is, antigens originating outside the cell in order to establish specific immunity or elicit humoral immunity. Blood, lymph, and body fluids circulate polypeptides derived from viruses, toxins, pollen, or bacteria. APC endocytosis these polypeptides into endosome, which then fuse with lysosomes whose acidic enzymes cleave the polypeptides into many different peptides. These peptides, typically between 13 and 17 amino acids in length are bound to the cleft of MHC-II glycoproteins and displayed as epitopes on the cell surface to CD4⁺ inducer (or helper) T cells. CD4⁺ helper T cell express both CD4 receptors as well as TCRs. When a CD4⁺ helper T cell's is naïve, its TCR can be imprinted by the epitope coupled within the MHC-II glycoprotein, thereby priming the naïve CD4⁺ helper T cell. Depending, in part, on the types of cytokines secreted by the APCs in the microenvironment, the primed CD4⁺ helper T cell (Th0) polarizes into either a memory Th cell, an effector Th cell of type 1 (Th1), type 2 (Th2), type 17 (Th17), or regulatory/suppressor (Treg). Thus, MHC class II helps mediate specific immunity, the primary means to activate antigen-specific effector Th cell and release of various cytokines in response to an antigen, and humoral immunity, the primary means to stimulate B-cells to elicit an antibody response against an antigen or immune tolerance of an antigen.

Currently, there is no permanent cure for gender-biased immune disorders. Existing medications include nonsteroidal anti-inflammatory drugs (NSAIDs), corticosteroids, antimalarial drugs, immunosuppressive or cytotoxic drugs, and B-cell-suppressing drugs. However, undesirable side effects are associated with each of these medications. For example, common side effects of NSAIDs are stomach upset, abdominal pain, ulcers, and even ulcer bleeding. Side effects of corticosteroids include weight gain, thinning of the bones and skin, infection, diabetes, facial puffiness, cataracts, and death (necrosis) of the tissues in large joints. Immunosuppressive or cytotoxic drugs can seriously depress blood-cell counts and increase risks of infection and bleeding, liver toxicity, and impair kidney function. In addition, these drugs cannot be taken during pregnancy or conception because of risk to the fetus.

Thus, there still exists a need for the development of pharmaceutical compositions and/or therapeutic compounds effective at treating gender-biased immune disorders.

### SUMMARY

Aspects of the present specification disclose compositions comprising a therapeutic compound capable of modulating activity of a hormone. Therapeutic compounds include, without limitation, a progesterone agonist, a selective progesterone receptor modulator (SPRM), an estrogen antagonist, a selective estrogen receptor modulator (SERM), an estrogen biosynthesis enzyme inhibitor, an estrogen agonist, an androgen agonist, a selective androgen receptor modulator (SARM), or any combination thereof. The composition disclosed herein may reduce an unwanted side and/or reduce a symptom associated with a gender-biased immune disorder.

Aspects of the present specification also disclose methods of treating a gender-biased immune disorder in an individual. The disclosed methods comprising the step of administering a pharmaceutical composition disclosed herein to an individual, wherein administration reduces a symptom associated with a gender-biased immune disorder. Administration of a pharmaceutical composition may also reduce an unwanted side in the individual.

Aspects of the present specification disclose uses of the disclosed compositions and/or therapeutic compounds in the manufacture of a medicament for the treatment of a gender-biased immune disorder.

Aspects of the present specification disclose uses of the disclosed compositions and/or therapeutic compounds in the treatment of a gender-biased immune disorder.

### BRIEF DESCRIPTION OF THE DRAWINGS

FIG. 1 shows the steroidogenesis pathway for sex hormones, including the enzymes involved in the pathway.

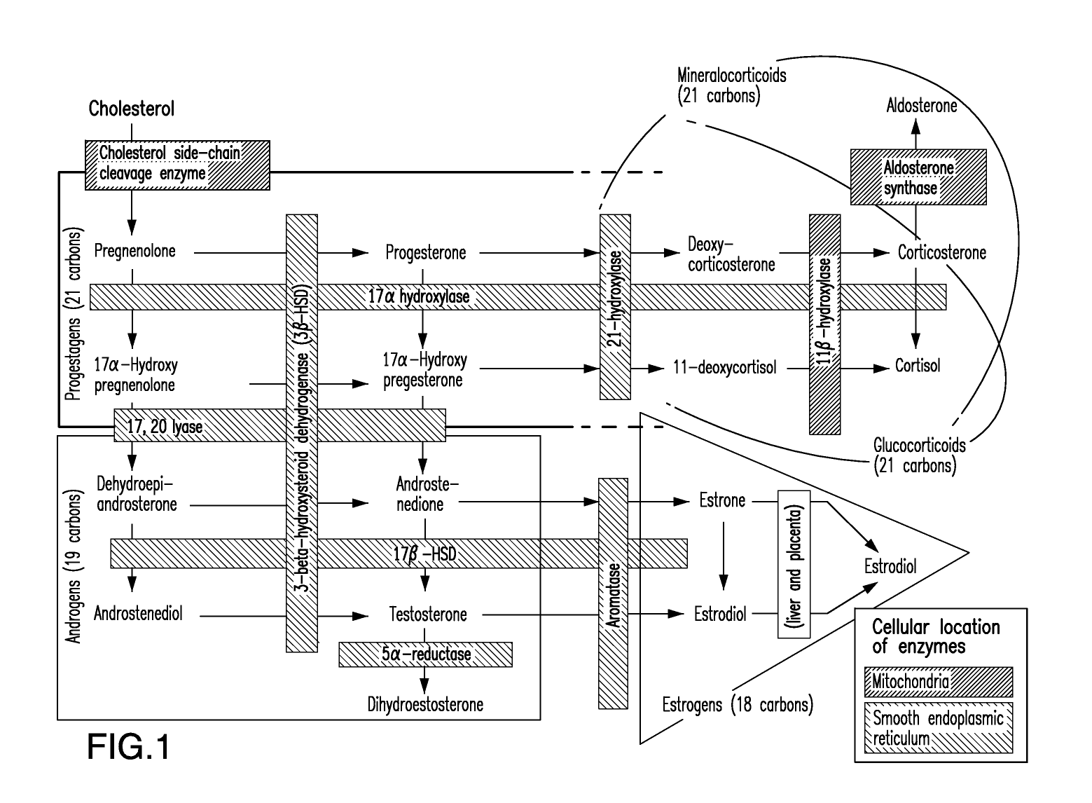

FIG. 2 shows the ability of Testosterone (FIG. 2A) or Progesterone (FIG. 2B) to modulate the response of antibodies to their target. 1A, Control Group (Formulation 1); 1B Control Group (Formulation 2); 2A, CH47 Group (Formulation 1); and 2B, CH47 Group (Formulation 2).

### DESCRIPTION

The present specification is based on the finding that presentation of self antigens is a normal occurrence but at very low levels that are below the thresehold of immune activation. For example, during apoptosis, nuclear proteins can be modified through molecular cleavage and redistributed on the cell surface. Under physiological conditions, these proteins do not induce any autoimmune response because they are rapidly removed by macrophages, and as such, no harm is caused due to induced self-tolerance or non-clinical outcomes. However, in circumstances where abnormal hormonal levels occur, the same antigens are processed and presented differently or in amounts above the thresehold for immune activation. For example, inappropriate clearance of apoptotic bodies due to abnormal hormonal levels can lead to the persistence of auto-antigens that can break tolerance and initiate production of autoantibodies. Under these pathophysiological conditions, these antigens induce an autoimmune response resulting in a loss of self-tolerance and manifestation of a disorder or disease.

One explanation for this effect could be that aberrant hormonal level increase expression or activity of proteolytic enzymes resulting in the same epitopes becoming immunodominant and/or trigger production or activation of a different set of enzymes that would cleave the protein on different sites generating new epitopes and a different level of response. As one non-limiting example, hormones could influence whether a self antigen is trafficked into the endogenous pathway used to associate peptide fragments with MHC-I glycoproteins or whether it is trafficked into the exogenous pathway used to associate peptide fragments with MHC-II glycoproteins. As another non-limiting example, hormones could alter expression of one or more enzymes present in proteosomes involved in the processing of antigens for MHC-I presentation and/or they could alter expression of one or more enzymes present in lysosomes involved in the processing of antigens for MHC-II presentation, which in turn could influence the levels an antigen is presented to B cells, CD8⁺ or CD4⁺ T cells respectively.

Autoimmunity is the failure of an organism in recognizing its own constituent parts as self, thus leading to an immune response against its own cells and tissues. Any disease that results from such an aberrant immune response is termed an autoimmune disease. Most autoimmune diseases are characterized by the presentation of antigens bound to MHC-II glycoproteins to other immune cells in a manner that induces a specific response against the antigen. This may confuse killer T-cells, which respond inappropriately by attacking these cells.

Strikingly, most autoimmune diseases are gender bias. For example, the incidence of Sjogren syndrome is 9 to 20 times more likely in females than males; the incidence of Hashimoto's thyroiditis is 10 times more likely in females as oppose to males; the incidence of primary biliary cirrhosis is 9 to 10 times more likely in females than males; the incidence of systemic lupus erythematosus (SLE) and antiphospholipid antibodies are each 9 times more likely in females than males; the incidence of mixed connective tissue disease, chronic active hepatitis and autoimmune thyrid disease are each 8 times more likely in females than males; the incidence of Graves' disease is 7 times more likely in females than males; the incidence of autoimmune hepatitis is 5 times more likely in females than males; the incidence of systemic sclerosis (scleroderma) is 3 to 5 times more likely in females than males; the incidence of rheumatoid arthritis is 3 to 4 times more likely in females than males; the incidence of myasthenia gravis and multiple sclerosis are each 2 to 3 times more likely in females than males; and the incidence of autoimmune thrombocytopenic purpura and dermatomyositis are each 2 times more likely in females than males. On the other hand certain autoimmune disease are more prevalent in males rather than females. For example, primary sclerosing cholangitis, ankylosing spondylitis and diabetes mellitus type 1 each occur in twice as many men as women. In addition, men are more likely than women to develop gastritis, Wegener's granulomatosis, Crohn's disease, autoimmune myocarditis and psoriasis. Although not an immune disorder, the incidence of Chagas disease is significantly greater in males as opposed to females

The present specification discloses that there is a tight balance of hormonal levels where estrogen typically has the opposite effects to progesterone. When the controls regulating this balance are disrupted, allowing hormaonal levels to shift one way or another, antigens that shouldn't be presented to immune cells are because they are processed differently. So a treatment that rebalances hormonal levels either by increasing one or lowering the other, or both, should return the processing of antigens to the normal non-reactive pathway or below the reactive threshold.

The present specification discloses compositions of various compounds that produce therapeutic effects in an individual suffering from a gender-biased immune disorder by reducing a symptom associated with a gender-biased immune disorder. The present specification discloses the finding that hormone treatment can be used to enhance the antigen processing pathway suppressed, disfavoured or otherwise underutilized in individuals suffering from a gender-biased immune disorder. Concomitant to this treatment, an additional hormone treatment can be used to suppress the antigen processing pathway causal to a gender-biased immune disorder. As such, the concept is to shift MHC-I and/or MHC-II antigen presentation to a beneficial state as a means of disease management where antibody production is a concern. For example, a compound can be administered to an individual suffering from a female-biased immune disorder, like SLE, that increases Progesterone and/or Testosterone levels, thereby returning intracellular antigen presentation to a non-activating one. In conjugation with this Progesterone and/or Testosterone treatment, a compound that decreases Estrogen levels can be concomitantly administered in order to maximize the effect of Progesterone and Testosterone. Similarly, a compound can be administered to an individual suffering from a male-biased immune disorder, like ankylosing spondylitis, that increases Estrogen levels, thereby returning intracellular antigen presentation to a non-activating one. In conjugation with or without this Estrogen treatment, a compound that decreases Progesterone and/or Testosterone levels can be concomitantly administered in order to maximize the effect of Estrogen.

It has long been known that hormones like Estrogen, Testosterone, Progesterone, modulate the secondary sex characteristics of an individual undergoing puberty. However, as disclosed herein, it has been discovered that short-term exposure to hormone also have the ability to modulate the immune system. Because this exposure is acute, being hours in duration, the sexual characteristics of the individual undergoing treatment remains unaffected. Thus, hormone therapies over short periods of time to trigger the immune response without triggering sexuality function has been found to be effective in treating a gender-biased immune disorder by suppressing the antigen processing pathway causal to a gender-biased immune disorder and/or enhance the antigen processing pathway suppressed by a gender-biased immune disorder, thereby shifting MHC I/-II antigen presentation to a beneficial state as a means of disease management.

Aspects of the present specification disclose, in part, a pharmaceutical composition. As used herein, the term “pharmaceutical composition” is synonymous with “pharmaceutically acceptable composition” and refers to a therapeutically effective concentration of an active ingredient, such as, e.g., any of the therapeutic compounds disclosed herein. As used herein, the term “pharmaceutically acceptable” refers to any molecular entity or composition that does not produce an adverse, allergic or other untoward or unwanted reaction when administered to an individual. A pharmaceutical composition disclosed herein is useful for medical and veterinary applications. A pharmaceutical composition may be administered to an individual alone, or in combination with other supplementary active ingredients, agents, drugs or hormones.

A pharmaceutical composition disclosed herein may comprise one or more therapeutic compounds disclosed herein. In one embodiment, pharmaceutical composition disclosed herein may comprise only a single a therapeutic compound capable of modulating activity of a hormone. In another embodiment, pharmaceutical composition disclosed herein may comprise a plurality of therapeutic compounds capable of modulating activity of a hormone. In aspects of this embodiment, a pharmaceutical composition disclosed herein comprises at least one therapeutic compound capable of modulating activity of a hormone, at least two therapeutic compounds capable of modulating activity of a hormone, at least three therapeutic compounds capable of modulating activity of a hormone, or at least four therapeutic compounds capable of modulating activity of a hormone. In other aspects of this embodiment, a pharmaceutical composition disclosed herein comprises at most two therapeutic compounds capable of modulating activity of a hormone, at most three therapeutic compounds capable of modulating activity of a hormone, or at most four therapeutic compounds capable of modulating activity of a hormone. In yet other aspects of this embodiment, a pharmaceutical composition disclosed herein comprises one to three therapeutic compounds capable of modulating activity of a hormone, two to four therapeutic compounds capable of modulating activity of a hormone, two to five therapeutic compounds capable of modulating activity of a hormone, three to five therapeutic compounds capable of modulating activity of a hormone, or two to three therapeutic compounds capable of modulating activity of a hormone. In aspects of this embodiment, a therapeutic compound capable of modulating activity of a hormone includes, without limitation, a progesterone agonist, a SPRM, a progesterone antagonist, an estrogen antagonist, a SERM, an estrogen biosynthesis enzyme inhibitor, an estrogen agonist, an androgen agonist, a SARM, an androgen antagonist, or any combination thereof.

A pharmaceutical composition disclosed herein may reduces the occurrence of an unwanted side effect elicited by administration of one or more of the therapeutic compounds contained in the pharmaceutical composition. Examples of an unwanted side effect, include, without limitation, masculinization, increased muscle mass, increased fat deposition, menorrhea, increased hair growth on face, torso, and/or extremities, feminization, mammary gland development, cancer, growth hormone effects, diabetes, mood swings, hair loss or growth on face, torso, and/or extremities, change in voice resonance or pitch, reduced sexual desire, reduced sexual arousal, reduced sexual orgasm, erectile dysfunction, impotence, infertility, or any combination thereof.

Aspects of the present specification disclose, in part, a therapeutic compound. A therapeutic compound is a compound that provides pharmacological activity or other direct effect in the diagnosis, cure, mitigation, treatment, or prevention of disease, or to affect the structure or any function of the body of man or animals. Any suitable form of a therapeutic compound may be chosen. A therapeutic compound disclosed herein may be used in the form of a pharmaceutically acceptable salt, solvate, or solvate of a salt, e.g. the hydrochloride. Additionally, therapeutic compound disclosed herein may be provided as racemates, or as individual enantiomers, including the R- or S-enantiomer. Thus, the therapeutic compound disclosed herein may comprise a R-enantiomer only, a S-enantiomer only, or a combination of both a R-enantiomer and a S-enantiomer of a therapeutic compound. A therapeutic compound disclosed herein may also be provided as prodrug or active metabolite.

A therapeutic compound disclosed herein may be capable of modulating activity of a hormone. As used herein, the term “capable of modulating activity of a hormone” refers to the ability of the therapeutic compound disclosed herein to directly or indirectly increase the level of a progesterone in an individual, directly or indirectly increase the level of an androgen in an individual, directly or indirectly decrease the level of an estrogen in an individual, or any combination thereof.

In an embodiment, a therapeutic compound disclosed herein has a hormone modulating activity capable of reducing the level and/or activity of an estrogen. Such reduction will typically decrease estrogen level and/or activity to a physiologically normal or base-line level/activity or decrease estrogen level and/or activity to below a physiologically normal or base-line level/activity. In an aspect of this embodiment, a therapeutic compound disclosed herein has a hormone modulating activity capable of reducing a level and/or activity of an estrone (E1), an estradiol (E2), an estriol (E3), estetrol (E4), or any combination thereof. In aspects of this embodiment, a therapeutic compound disclosed herein has a hormone modulating activity capable of reducing the level and/or activity of an estrogen by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In other aspects of this embodiment, a therapeutic compound disclosed herein has a hormone modulating activity capable of reducing the level and/or activity of an estrogen by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90% or at most 95%. In yet other aspects of this embodiment, a therapeutic compound disclosed herein has a hormone modulating activity capable of reducing the level and/or activity of an estrogen in a range from, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In an embodiment, a therapeutic compound disclosed herein has a hormone modulating activity capable of reducing a level and/or activity of a biosynthetic enzyme for an estrogen. Such reduction will typically decrease a biosynthetic enzyme level and/or activity to a physiologically normal or base-line level and/or activity or decrease a biosynthetic enzyme level and/or activity to below a physiologically normal or base-line level/activity. In an aspect of this embodiment, a therapeutic compound disclosed herein has a hormone modulating activity capable of reducing the level and/or activity of a 3-β-hydroxysteroid dehydrogenase, an aromatase, or any combination thereof. In aspects of this embodiment, a therapeutic compound disclosed herein has a hormone modulating activity capable of reducing a level and/or activity of a biosynthetic enzyme for an estrogen by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In other aspects of this embodiment, a therapeutic compound disclosed herein has a hormone modulating activity capable of reducing a level and/or activity of a biosynthetic enzyme for an estrogen by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90% or at most 95%. In yet other aspects of this embodiment, a therapeutic compound disclosed herein has a hormone modulating activity capable of reducing a level and/or activity of a biosynthetic enzyme for an estrogen in a range from, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In an embodiment, a therapeutic compound disclosed herein has a hormone modulating activity capable of increasing the level and/or activity of an estrogen. Such increase will typically elevate estrogen level and/or activity to a physiologically normal or base-line level/activity or elevate estrogen level and/or activity above a physiologically normal or base-line level/activity. In an aspect of this embodiment, a therapeutic compound disclosed herein has a hormone modulating activity capable of increasing a level and/or activity of an estrone (E1), an estradiol (E2), an estriol (E3), estetrol (E4), or any combination thereof. In aspects of this embodiment, a therapeutic compound disclosed herein has a hormone modulating activity capable of increasing the level and/or activity of an estrogen by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In other aspects of this embodiment, a therapeutic compound disclosed herein has a hormone modulating activity capable of increasing the level and/or activity of an estrogen by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90% or at most 95%. In yet other aspects of this embodiment, a therapeutic compound disclosed herein has a hormone modulating activity capable of increasing the level and/or activity of an estrogen in a range from, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In an embodiment, a therapeutic compound disclosed herein has a hormone modulating activity capable of increasing a level and/or activity of a biosynthetic enzyme for an estrogen. Such increase will typically elevate a biosynthetic enzyme level and/or activity to a physiologically normal or base-line level and/or activity or elevate a biosynthetic enzyme level and/or activity above a physiologically normal or base-line level/activity. In an aspect of this embodiment, a therapeutic compound disclosed herein has a hormone modulating activity capable of increasing the level and/or activity of a 3-β-hydroxysteroid dehydrogenase, an aromatase, or any combination thereof. In aspects of this embodiment, a therapeutic compound disclosed herein has a hormone modulating activity capable of increasing a level and/or activity of a biosynthetic enzyme for an estrogen by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In other aspects of this embodiment, a therapeutic compound disclosed herein has a hormone modulating activity capable of increasing a level and/or activity of a biosynthetic enzyme for an estrogen by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90% or at most 95%. In yet other aspects of this embodiment, a therapeutic compound disclosed herein has a hormone modulating activity capable of increasing a level and/or activity of a biosynthetic enzyme for an estrogen in a range from, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In an embodiment, a therapeutic compound disclosed herein has a hormone modulating activity capable of reducing a level and/or activity of an androgen. Such reduction will typically decrease androgen levels to a physiologically normal or base-line level or decrease androgen level and/or activity to below a physiologically normal or base-line level/activity. In an aspect of this embodiment, a therapeutic compound disclosed herein has a hormone modulating activity capable of reducing the levels of an Androstenediol, an Androstenedione, an Androsterone, a Dehydroepiandrosterone (DHEA), a Dihydrotestosterone (DHT), a Testosterone, or any combination thereof. In aspects of this embodiment, a therapeutic compound disclosed herein has a hormone modulating activity capable of reducing the level and/or activity of an androgen by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In other aspects of this embodiment, a therapeutic compound disclosed herein has a hormone modulating activity capable of reducing the level and/or activity of an androgen by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90% or at most 95%. In yet other aspects of this embodiment, a therapeutic compound disclosed herein has a hormone modulating activity capable of reducing the level and/or activity of an androgen in a range from, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In an embodiment, a therapeutic compound disclosed herein has a hormone modulating activity capable of reducing a level and/or activity of a biosynthetic enzyme for an androgen. Such reduction will typically decrease biosynthetic enzyme level and/or activity to a physiologically normal or base-line level/activity or decrease a biosynthetic enzyme level and/or activity to below a physiologically normal or base-line level and/or activity. In an aspect of this embodiment, a therapeutic compound disclosed herein has a hormone modulating activity capable of reducing the level and/or activity of a 17α hydeoxylase, a 17α-hydroxypregesterone, a 17,20 lyase, a 3-β-hydroxysteroid dehydrogenase, a 5α reductase, or any combination thereof. In aspects of this embodiment, a therapeutic compound disclosed herein has a hormone modulating activity capable of reducing a level and/or activity of a biosynthetic enzyme for an androgen by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In other aspects of this embodiment, a therapeutic compound disclosed herein has a hormone modulating activity capable of reducing a level and/or activity of a biosynthetic enzyme for an androgen by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90% or at most 95%. In yet other aspects of this embodiment, a therapeutic compound disclosed herein has a hormone modulating activity capable of reducing a level and/or activity of a biosynthetic enzyme for an androgen in a range from, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In an embodiment, a therapeutic compound disclosed herein has a hormone modulating activity capable of increasing a level and/or activity of an androgen. Such increase will typically elevate androgen levels to a physiologically normal or base-line level or elevate androgen level and/or activity above a physiologically normal or base-line level/activity. In an aspect of this embodiment, a therapeutic compound disclosed herein has a hormone modulating activity capable of increasing the levels of an Androstenediol, an Androstenedione, an Androsterone, a Dehydroepiandrosterone (DHEA), a Dihydrotestosterone (DHT), a Testosterone, or any combination thereof. In aspects of this embodiment, a therapeutic compound disclosed herein has a hormone modulating activity capable of increasing the level and/or activity of an androgen by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In other aspects of this embodiment, a therapeutic compound disclosed herein has a hormone modulating activity capable of increasing the level and/or activity of an androgen by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90% or at most 95%. In yet other aspects of this embodiment, a therapeutic compound disclosed herein has a hormone modulating activity capable of increasing the level and/or activity of an androgen in a range from, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In an embodiment, a therapeutic compound disclosed herein has a hormone modulating activity capable of increasing a level and/or activity of a biosynthetic enzyme for an androgen. Such increase will typically elevate a biosynthetic enzyme level and/or activity to a physiologically normal or base-line level/activity or elevate a biosynthetic enzyme level and/or activity above a physiologically normal or base-line level and/or activity. In an aspect of this embodiment, a therapeutic compound disclosed herein has a hormone modulating activity capable of increasing the level and/or activity of a 17α hydeoxylase, a 17α-hydroxypregesterone, a 17,20 lyase, a 3-β-hydroxysteroid dehydrogenase, a 5α reductase, or any combination thereof. In aspects of this embodiment, a therapeutic compound disclosed herein has a hormone modulating activity capable of increasing a level and/or activity of a biosynthetic enzyme for an androgen by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In other aspects of this embodiment, a therapeutic compound disclosed herein has a hormone modulating activity capable of increasing a level and/or activity of a biosynthetic enzyme for an androgen by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90% or at most 95%. In yet other aspects of this embodiment, a therapeutic compound disclosed herein has a hormone modulating activity capable of increasing a level and/or activity of a biosynthetic enzyme for an androgen in a range from, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In an embodiment, a therapeutic compound disclosed herein has a hormone modulating activity capable of reducing a level and/or activity of a progesterone. Such reduction will typically decrease progesterone levels to a physiologically normal or base-line level or decrease progesterone level and/or activity below a physiologically normal or base-line level and/or activity. In an aspect of this embodiment, a therapeutic compound disclosed herein has a hormone modulating activity capable of reducing the level and/or activity of a 17-hydroxyprogesterone, a 17-hydroxypregnenolone, a pregnenolone, a progesterone, or any combination thereof. In aspects of this embodiment, a therapeutic compound disclosed herein has a hormone modulating activity capable of reducing the level and/or activity of a progesterone by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In other aspects of this embodiment, a therapeutic compound disclosed herein has a hormone modulating activity capable of reducing the level and/or activity of a progesterone by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90% or at most 95%. In yet other aspects of this embodiment, a therapeutic compound disclosed herein has a hormone modulating activity capable of reducing the level and/or activity of a progesterone in a range from, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In an embodiment, a therapeutic compound disclosed herein has a hormone modulating activity capable of reducing a level and/or activity of a biosynthetic enzyme for a progesterone. Such reduction will typically decrease a biosynthetic enzyme level and/or activity to a physiologically normal or base-line level/activity or decrease a biosynthetic enzyme level and/or activity below a physiologically normal or base-line level and/or activity. In an aspect of this embodiment, a therapeutic compound disclosed herein has a hormone modulating activity capable of reducing the level and/or activity of a cholesterol side-chain cleavage enzyme, a 17α hydroxylase, a 3-β-hydroxysteroid dehydrogenase, or any combination thereof. In aspects of this embodiment, a therapeutic compound disclosed herein has a hormone modulating activity capable of reducing a level and/or activity of a biosynthetic enzyme for a progesterone by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In other aspects of this embodiment, a therapeutic compound disclosed herein has a hormone modulating activity capable of reducing a level and/or activity of a biosynthetic enzyme for a progesterone by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90% or at most 95%. In yet other aspects of this embodiment, a therapeutic compound disclosed herein has a hormone modulating activity capable of reducing a level and/or activity of a biosynthetic enzyme for a progesterone in a range from, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In an embodiment, a therapeutic compound disclosed herein has a hormone modulating activity capable of increasing a level and/or activity of a progesterone. Such increase will typically elevate progesterone levels to a physiologically normal or base-line level or elevate progesterone level and/or activity above a physiologically normal or base-line level and/or activity. In an aspect of this embodiment, a therapeutic compound disclosed herein has a hormone modulating activity capable of increasing the level and/or activity of a 17-hydroxyprogesterone, a 17-hydroxypregnenolone, a pregnenolone, a progesterone, or any combination thereof. In aspects of this embodiment, a therapeutic compound disclosed herein has a hormone modulating activity capable of increasing the level and/or activity of a progesterone by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In other aspects of this embodiment, a therapeutic compound disclosed herein has a hormone modulating activity capable of increasing the level and/or activity of a progesterone by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90% or at most 95%. In yet other aspects of this embodiment, a therapeutic compound disclosed herein has a hormone modulating activity capable of increasing the level and/or activity of a progesterone in a range from, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In an embodiment, a therapeutic compound disclosed herein has a hormone modulating activity capable of increasing a level and/or activity of a biosynthetic enzyme for a progesterone. Such increase will typically elevate a biosynthetic enzyme level and/or activity to a physiologically normal or base-line level/activity or elevate a biosynthetic enzyme level and/or activity above a physiologically normal or base-line level and/or activity. In an aspect of this embodiment, a therapeutic compound disclosed herein has a hormone modulating activity capable of increasing the level and/or activity of a cholesterol side-chain cleavage enzyme, a 17α hydroxylase, a 3-β-hydroxysteroid dehydrogenase, or any combination thereof. In aspects of this embodiment, a therapeutic compound disclosed herein has a hormone modulating activity capable of increasing a level and/or activity of a biosynthetic enzyme for a progesterone by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In other aspects of this embodiment, a therapeutic compound disclosed herein has a hormone modulating activity capable of increasing a level and/or activity of a biosynthetic enzyme for a progesterone by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90% or at most 95%. In yet other aspects of this embodiment, a therapeutic compound disclosed herein has a hormone modulating activity capable of increasing a level and/or activity of a biosynthetic enzyme for a progesterone in a range from, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In another embodiment, a therapeutic compound disclosed herein reduces a level and/or activity of a gender-biased immune disorder-causing antigen. A gender-biased immune disorder-causing antigen can be one which elicits an immune response from a cytotoxic T lymphocyte (CTL), CD8⁺ T cell and/or CD4⁺ T cell, and thus can be a cytotoxic T lymphocyte (CTL) antigen, CD8⁺ T cell antigen and/or CD4⁺ T cell antigen. A gender-biased immune disorder-causing antigen can be a MHC II antigen. Examples of a gender-biased immune disorder-causing antigen include. Without exception a peptide, a polypeptide, a nucleic acid, or polynucleic acid produced by an individual. In aspects of this embodiment, a therapeutic compound disclosed herein reduces a level and/or activity of a gender-biased immune disorder-causing antigen by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In other aspects of this embodiment, a therapeutic disclosed herein reduces a level and/or activity of a gender-biased immune disorder-causing antigen by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In another embodiment, a therapeutic compound disclosed herein reduces antigenicity of a gender-biased immune disorder-causing antigen. In aspects of this embodiment, a therapeutic compound disclosed herein reduces an antigenicity of a gender-biased immune disorder-causing antigen by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In other aspects of this embodiment, a therapeutic disclosed herein reduces antigenicity of a gender-biased immune disorder-causing antigen by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In another embodiment, a therapeutic compound disclosed herein reduces the ability of an immune system to react to a gender-biased immune disorder-causing antigen. In aspects of this embodiment, a therapeutic compound disclosed herein reduces the ability of an immune system to react to a gender-biased immune disorder-causing antigen by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In other aspects of this embodiment, a therapeutic disclosed herein reduces the ability of an immune system to react to a gender-biased immune disorder-causing antigen by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In another embodiment, a therapeutic compound disclosed herein reduces the ability of an immune system to react to a SLE-causing antigen and/or an α-SLE antibody. In aspects of this embodiment, a therapeutic compound disclosed herein reduces the ability of an immune system to react to a SLE causing antigen and/or an α-SLE antibody by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In other aspects of this embodiment, a therapeutic disclosed herein reduces the ability of an immune system to react to a SLE causing antigen and/or an α-SLE antibody by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In another embodiment, a therapeutic compound disclosed herein reduces antigenicity of a P1/P2 antigen and/or an α-P1/P2 ribosomal protein antibody. In aspects of this embodiment, a therapeutic compound disclosed herein reduces antigenicity a P1/P2 antigen and/or an α-P1/P2 ribosomal protein antibody by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In other aspects of this embodiment, a therapeutic compound disclosed herein reduces antigenicity of a P1/P2 antigen and/or an α-P1/P2 ribosomal protein antibody by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In another embodiment, a therapeutic compound disclosed herein reduces antigenicity of a ssDNA antigen and/or an α-ssDNA antibody. In aspects of this embodiment, a therapeutic compound disclosed herein reduces antigenicity a ssDNA antigen and/or an α-ssDNA antibody by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In other aspects of this embodiment, a therapeutic compound disclosed herein reduces antigenicity of a ssDNA antigen and/or an α-ssDNA antibody by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In another embodiment, a therapeutic compound disclosed herein reduces apoptosis. In aspects of this embodiment, a therapeutic compound disclosed herein reduces apoptosis by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In other aspects of this embodiment, a therapeutic compound disclosed herein reduces apoptosis by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In an embodiment, a compound capable of modulating activity of a hormone is a progesterone agonist. Progesterone agonist are a group of naturally-occurring hormones or chemically-synthesized compound that function in maintaining pregnancy (pro-gestational), although they are also present at other phases of the estrous and menstrual cycle. Characterized by its basic 21-carbon skeleton, called a pregnane skeleton, a progesterone agonist, includes both endogenous and exogenous naturally occurring compounds. Synthetic progesterone agonist can be based upon a pregnane skeleton or structures not derived from this skeleton. Progesterone also known as P4 (pregn-4-ene-3,20-dione) is the major endogenous naturally occurring human progesterone agonist. Progestins refer to synthetic progesterone agonists, or exogenous naturally occurring progesterone agonists, that have effects similar to progesterone.

A progesterone agonist mediates its effects through a progesterone receptor (PR, also known as NR3C3 or nuclear receptor subfamily 3, group C, member 3), a nuclear receptor found inside cells. In humans, PR is encoded by a single PGR gene residing on chromosome 11q22, and it produces two main forms, A and B, that differ in their molecular weight. In common with other steroid receptors, a PR comprises a N-terminal NB domain is able to transactivate gene transcription in the absence of bound ligand. While this region is able to activate gene transcription without ligand, this activation is weak and more selective compared to the activation provided by the E domain. The C domain, also known as the DNA-binding domain, includes a highly-conserved zinc-finger that binds to target DNA sequences called progesterone-responsive elements (PRE). The D domain is a hinge region that connects the C and E domains. The E domain contains the ligand binding cavity as well as binding sites for coactivator and corepressor proteins. The E-domain in the presence of bound ligand is able to activate gene transcription. The C-terminal F domain function is not entirely clear and is variable in length. A special transcription activation function (TAF), called TAF-3, is present in the progesterone receptor-B, in a B-upstream segment (BUS) at the amino acid terminal. This segment is not present in the receptor-A. PRs function as dimeric molecules in nuclei to regulate the transcription of target genes in a ligand-responsive manner.

Progesterone agonist binding is necessary to induce PR activity. In the absence of androgen agonist binding, the carboxyl terminal end of PR inhibits transcription, even though this receptor weakly associated with nuclear components. After progesterone agonist binding, PR restructuring with dimerization follows that 1) induces a structural change that removes the inhibitory action; 2) greatly increases receptor affinity for the PRE; and 3) the complex enters the nucleus and specifically binds to DNA at the PREs. There transcription takes place, resulting in formation of messenger RNA that is translated by ribosomes to produce specific proteins. Examples of suitable progesterone agonists include, without limitation, 11-Dehydroprogesterone, 11-Deoxycorticosterone, 17-Hydroxyprogesterone, 19-Norprogesterone, Algestone, Algestone acetonide, Algestone acetophenide, Allylestrenol, Altrenogest, Amadinone, Amadinone acetate, Anagestone, Anagestone acetate, Chlormadinone, Chlormadinone acetate, Cingestol, Cismadinone, Cismadinone acetate, Clogestone, Clogestone acetate, Clomegestone, Cyproterone, Cyproterone acetate, Delmadinone, Delmadinone acetate, Demegestone, Deoxycorticosterone, Desogestrel, Dienogest, Dimethisterone, Drospirenone, Dydrogesterone, Edogesterone, Ethisterone, Ethynerone, Ethynodiol, Ethynodiol diacetate, Etonogestrel, Etynodiol, Flugestone, Flugestone acetate, Gestaclone, Gestadienol, Gestodene, Gestonorone, Gestonorone caproate, Gestovister, Gestrinone, Gestronol, Haloprogesterone, Hydromadinone, Hydroxyprogesterone acetate, Hydroxyprogesterone caproate, Hydroxyprogesterone heptanoate, Hydroxyprogesterone hexanoate, Levonorgestrel, Lutenyl, Lynestrenol, Med rogestone, Medroxyprogesterone, Medroxyprogesterone acetate, Megestrol, Megestrol acetate, Melengestrol, Melengestrol acetate, Methylestrenolone, Methynodiol, Methynodiol diacetate, Metogest, Nandrolone, Nestorone, Nomegestrol, Nomegestrol acetate, Norelgestromin, Norethindrone, Norethindrone acetate, Norethisterone, Norethisterone acetate, Norethisterone acetate oxime, Norethisterone enanthate, Norethynodrel, Norethynodrel diacetate, Norgesterone, Norgestimate, Norgestomet, Norgestrel, Norgestrienone, Norvinisterone, Org-2058, Oxogestone, Oxogestone phenpropionate, Pentagestrone, Pregnane, Progesterone, Proligestone, Promegestone, Quingestanol, Quingestanol acetate, Quingestrone, Segesterone, Spironenone, Spironolactone, Tanaproget, Tibolone, Tigestol, Tosagestin, Trengestone, Trimegestone, and ZM-182,345.

A progesterone agonist may be a full agonist or a partial agonist. A full progesterone agonist is able to activate a PR and result in a maximal biological response. The natural endogenous ligand with the greatest efficacy for a given receptor is by definition a full agonist (100% efficacy). In aspects of this embodiment, a full progesterone agonist activates a PR and results in a biological response that is, e.g., about 90%, about 91%, about 92%, about 93%, about 94%, about 95%, about 96%, about 97%, about 98%, about 99%, or about 100% of a maximal biological response elicited by progesterone. In other aspects of this embodiment, a full progesterone agonist activates a PR and results in a biological response that is, e.g., at least 90%, at least 91%, at least 92%, at least 93%, at least 94%, at least 95%, at least 96%, at least 97%, at least 98%, or at least 99% of a maximal biological response elicited by progesterone. In yet other aspects of this embodiment, a full progesterone agonist activates a PR and results in a biological response that is between, e.g., about 90% to about 98%, about 91% to about 98%, about 92% to about 98%, about 93% to about 98%, about 94% to about 98%, about 95% to about 98%, about 90% to about 100%, about 91% to about 100%, about 92% to about 100%, about 93% to about 100%, about 94% to about 100%, or about 95% to about 100% of a maximal biological response elicited by progesterone.

Although activating a PR, a partial progesterone agonist does not fully activate the receptor, causing responses which are partial compared to a full agonist. In aspects of this embodiment, a partial progesterone agonist activates a PR and results in a biological response that is, e.g., about 40%, about 45% about 50%, about 55%, about 60%, about 65%, about 70%, about 75%, about 80%, about 85%, or about 90% of a maximal biological response elicited by progesterone. In other aspects of this embodiment, a partial progesterone agonist activates a PR and results in a biological response that is, e.g., at least 40%, at least 45% at least 50%, at least 55%, at least 60%, at least 65%, at least 70%, at least 75%, at least 80%, at least 85%, or at least 90% of a maximal biological response elicited by progesterone. In yet other aspects of this embodiment, a partial progesterone agonist activates a PR and results in a biological response that is, e.g., at most 40%, at most 45% at most 50%, at most 55%, at most 60%, at most 65%, at most 70%, at most 75%, at most 80%, at most 85%, or at most 90% of a maximal biological response elicited by progesterone. In still other aspects of this embodiment, a partial progesterone agonist activates a PR and results in a biological response that is between, e.g., about 40% to about 85%, about 45% to about 85%, about 50% to about 85%, about 55% to about 85%, about 60% to about 85%, about 65% to about 85%, about 70% to about 85%, about 75% to about 85%, about 80% to about 85%, about 40% to about 90%, about 45% to about 90%, about 50% to about 90%, about 55% to about 90%, about 60% to about 90%, about 65% to about 90%, about 70% to about 90%, about 75% to about 90%, about 80% to about 90%, or about 85% to about 90% of a maximal biological response elicited by progesterone.

In another embodiment, a compound capable of modulating activity of a hormone is a selective progesterone receptor modulator (SPRM). A SPRM differs in chemical structure from the endogenous hormone progesterone, but nevertheless bind to the same PR. A SPRM has the ability to promote PR interactions with different proteins such as transcriptional coactivator or corepressors. Furthermore, the ratio of coactivator to corepressor varies in different tissues. As a consequence, the same SPRM may be an agonist in some tissue (where coactivators predominate) while antagonistic in other tissues (where corepressors dominate). Examples of suitable SPRMs include, without limitation, Asoprisnil, Asoprisnil ecamate, J1042, LG-120,838, and Telapristone.

In aspects of this embodiment, a SPRM activates a PR and results in a biological response that is, e.g., about 5%, about 10%, about 15% about 20%, about 25%, about 30%, about 35%, about 40%, about 45%, about 50%, about 55%, about 60%, about 65%, about 70%, about 75%, about 80%, about 85%, about 90%, about 95%, or about 100% of a maximal biological response elicited by progesterone. In other aspects of this embodiment, a SPRM activates a PR and results in a biological response that is, e.g., at least 5%, at least 10%, at least 15% at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of a maximal biological response elicited by progesterone. In yet other aspects of this embodiment, a SPRM activates a PR and results in a biological response that is, e.g., at most 5%, at most 10%, at most 15% at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, or at most 95% of a maximal biological response elicited by progesterone. In still other aspects of this embodiment, a SPRM activates a PR and results in a biological response that is, e.g., about 5% to about 100%, about 10% to about 100%, about 15% to about 100%, about 20% to about 100%, about 25% to about 100%, about 30% to about 100%, about 35% to about 100%, about 40% to about 100%, about 45% to about 100%, or about 50% to about 100% of a maximal biological response elicited by progesterone.

In another embodiment, a compound capable of modulating activity of a hormone is a progesterone antagonist. Characterized by its basic 21-carbon skeleton, called a pregnane skeleton, a progesterone antagonist, includes both endogenous and exogenous naturally occurring compounds. Synthetic progesterone agonist can be based upon a pregnane skeleton or structures not derived from this skeleton.

Like a progesterone agonists disclosed herein, a progesterone antagonist mediates its effects through a progesterone receptor (PR). Progesterone antagonist binding is necessary to suppress PR activity. Examples of suitable progesterone antagonists include, without limitation, Aglepristone, Lilopristone, Lonaprisan, Mifepristone, Onapristone, Toripristone, ZM-150,271, and ZM-172,406.

A progesterone antagonist mediates its effects through a PR and may be a full antagonist, a partial antagonist, or an inverse agonist. A full progesterone antagonist binds to a PR, but does not activate the nuclear hormone receptor. This results in receptor blockage, inhibiting the binding of progesterone agonists, progesterone partial agonist, and/or progesterone inverse agonists.

In aspects of this embodiment, a full progesterone antagonist blocks a PR activity that results in a biological response that is, e.g., about 0%, about 1%, about 2%, about 3%, about 4%, about 5%, about 6%, about 7%, about 8%, about 9%, or about 10% of a maximal biological response elicited by progesterone. In other aspects of this embodiment, a full progesterone antagonist blocks a PR activity that results in a biological response that is, e.g., at most 1%, at most 2%, at most 3%, at most 4%, at most 5%, at most 6%, at most 7%, at most 8%, at most 9%, or at most 10% of a maximal biological response elicited by progesterone. In yet other aspects of this embodiment, a full progesterone antagonist blocks a PR activity that results in a biological response that is between, e.g., about 0% to about 5%, about 0% to about 6%, about 0% to about 7%, about 0% to about 8%, about 0% to about 9%, about 0% to about 10%, about 1% to about 5%, about 1% to about 6%, about 1% to about 7%, about 1% to about 8%, about 1% to about 9%, about 1% to about 10%, about 2% to about 5%, about 2% to about 6%, about 2% to about 7%, about 2% to about 8%, about 2% to about 9%, or about 2% to about 10% of a maximal biological response elicited by progesterone.

Although blocking a PR, a partial progesterone antagonist does not fully block receptor activity, causing responses which are partial compared to a full agonist. In aspects of this embodiment, a partial progesterone antagonist blocks a PR activity that results in a biological response that is, e.g., about 10%, about 15% about 20%, about 25%, about 30%, about 35%, or about 40% of a maximal biological response elicited by progesterone. In other aspects of this embodiment, a partial progesterone antagonist blocks a PR activity that results in a biological response that is, e.g., at most 10%, at most 15% at most 20%, at most 25%, at most 30%, at most 35%, or at most 40% of a maximal biological response elicited by progesterone. In yet other aspects of this embodiment, a partial progesterone antagonist blocks a PR activity that results in a biological response that is between, e.g., about 10% to about 20%, about 10% to about 25%, about 10% to about 30%, about 10% to about 35%, about 10% to about 40%, about 15% to about 20%, about 15% to about 25%, about 15% to about 30%, about 15% to about 35%, about 15% to about 40%, about 20% to about 25%, about 20% to about 30%, about 20% to about 35%, or about 20% to about 40% of a maximal biological response elicited by progesterone.

In another embodiment, a compound capable of modulating activity of a hormone is an estrogen antagonist. Estrogens are a group of compounds named for their importance in the estrous cycle of humans and other animals. They are the primary female sex hormones. Natural estrogens are steroid hormones, while some synthetic ones are non-steroidal. Characterized by its basic 18-carbon skeleton, called an estrane skeleton, an estrogen antagonist, includes both endogenous and exogenous naturally occurring compounds. Synthetic estrogen antagonists can be based upon an estrane skeleton or structures not derived from this skeleton.

The three major naturally occurring estrogens in women are estrone (E1), estradiol (E2), and estriol (E3). They make up 10-20%, 10-30%, and 60-80% of circulating estrogens, respectively. Though estriol is the most plentiful of the three estrogens it is also the weakest, whereas estradiol is the strongest with a potency of approximately 80× that of estriol. Thus, estradiol is the most important estrogen in non-pregnant females who are between the menarche and menopause stages of life. However, during pregnancy this role shifts to estriol, and in postmenopausal women estrone becomes the primary form of estrogen in the body. Another type of estrogen called estetrol (E4) is produced only during pregnancy. All of the different forms of estrogen are synthesized from androgens, specifically testosterone and androstenedione, by the enzyme aromatase.

The actions of estrogen are mediated by the estrogen receptor (ER), a dimeric nuclear protein that binds to DNA and controls gene expression. Like other steroid hormones, estrogen enters passively into the cell where it binds to and activates the estrogen receptor. The estrogen:ER complex binds to specific DNA sequences called a Estrogen Response Element (ERE) to activate the transcription of some 137 ER-regulated genes, of which 89 are direct target genes. However, it also has additional functions independent of DNA binding. Since estrogen enters all cells, its action is dependent on the presence of the ER in the cell. The ER is expressed in specific tissues including the ovary, uterus and breast.

In humans, there are two different forms of the estrogen receptor, usually referred to as α and β, each encoded by a separate gene (ESR1 and ESR2, respectively). ERs form dimers, and, since the two forms are coexpressed in many cell types, the receptors may form ERα (αα) or ERβ (ββ) homodimers or ERαβ (αβ) heterodimers. Estrogen receptor alpha and beta show significant overall sequence homology, and both are composed of five domains (listed from the N- to C-terminus; amino acid sequence numbers refer to human ER):(A-F domain). The N-terminal NB domain is able to transactivate gene transcription in the absence of bound ligand. While this region is able to activate gene transcription without ligand, this activation is weak and more selective compared to the activation provided by the E domain. The C domain, also known as the DNA-binding domain, binds to EREs in DNA. The D domain is a hinge region that connects the C and E domains. The E domain contains the ligand binding cavity as well as binding sites for coactivator and corepressor proteins. The E-domain in the presence of bound ligand is able to activate gene transcription. The C-terminal F domain function is not entirely clear and is variable in length. Due to alternative RNA splicing, several ER isoforms are known to exist. At least three ERα and five ERβ isoforms have been identified. The ERβ isoforms receptor subtypes can transactivate transcription only when as a heterodimer with ERβ1. Examples of suitable estrogen antagonist include, without limitation, 2-Hydroxyestrone, 16-Hydroxyestrone, Acefluranol, Clometerone, Delmadinone, Dimepregnen, Epitiostanol, Fulvestrant, Gestrinone, ICI-164,384, Mepitiostane, MPP, PHTPP, RU-58,668, SS1020, ZK-164,015, and ZK-191,703.

An estrogen antagonist mediates its effects through an ER and may be a full antagonist, a partial antagonist, or an inverse agonist. A full estrogen antagonist binds to an ER, but does not activate the nuclear hormone receptor. This results in receptor blockage, inhibiting the binding of estrogen agonists, estrogen partial agonist, and/or estrogen inverse agonists.

In aspects of this embodiment, a full estrogen antagonist blocks an ER activity that results in a biological response that is, e.g., about 0%, about 1%, about 2%, about 3%, about 4%, about 5%, about 6%, about 7%, about 8%, about 9%, or about 10% of a maximal biological response elicited by estrogen. In other aspects of this embodiment, a full estrogen antagonist blocks an ER activity that results in a biological response that is, e.g., at most 1%, at most 2%, at most 3%, at most 4%, at most 5%, at most 6%, at most 7%, at most 8%, at most 9%, or at most 10% of a maximal biological response elicited by estrogen. In yet other aspects of this embodiment, a full estrogen antagonist blocks an ER activity that results in a biological response that is between, e.g., about 0% to about 5%, about 0% to about 6%, about 0% to about 7%, about 0% to about 8%, about 0% to about 9%, about 0% to about 10%, about 1% to about 5%, about 1% to about 6%, about 1% to about 7%, about 1% to about 8%, about 1% to about 9%, about 1% to about 10%, about 2% to about 5%, about 2% to about 6%, about 2% to about 7%, about 2% to about 8%, about 2% to about 9%, or about 2% to about 10% of a maximal biological response elicited by estrogen.

Although blocking an ER, a partial estrogen antagonist does not fully block receptor activity, causing responses which are partial compared to a full agonist. In aspects of this embodiment, a partial estrogen antagonist blocks an ER activity that results in a biological response that is, e.g., about 10%, about 15% about 20%, about 25%, about 30%, about 35%, or about 40% of a maximal biological response elicited by estrogen. In other aspects of this embodiment, a partial estrogen antagonist blocks an ER activity that results in a biological response that is, e.g., at most 10%, at most 15% at most 20%, at most 25%, at most 30%, at most 35%, or at most 40% of a maximal biological response elicited by estrogen. In yet other aspects of this embodiment, a partial estrogen antagonist blocks an ER activity that results in a biological response that is between, e.g., about 10% to about 20%, about 10% to about 25%, about 10% to about 30%, about 10% to about 35%, about 10% to about 40%, about 15% to about 20%, about 15% to about 25%, about 15% to about 30%, about 15% to about 35%, about 15% to about 40%, about 20% to about 25%, about 20% to about 30%, about 20% to about 35%, or about 20% to about 40% of a maximal biological response elicited by estrogen.

An inverse estrogen agonist reduces an activity of an ER by inhibiting their constitutive or baseline activity (negative efficacy). In aspects of this embodiment, an inverse estrogen antagonist reduces a constitutive or baseline ER activity by, e.g., about 5%, about 10%, about 15% about 20%, about 25%, about 30%, about 35%, about 40%, about 45%, about 50%, about 55%, about 60%, about 65%, about 70%, about 75%, about 80%, about 85%, about 90%, about 95%, or about 100% from the constitutive or baseline activity. In other aspects of this embodiment, an inverse estrogen antagonist reduces a constitutive or baseline ER activity by, e.g., at least 5%, at least 10%, at least 15% at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% from the constitutive or baseline activity. In yet other aspects of this embodiment, an inverse estrogen antagonist reduces a constitutive or baseline ER activity by, e.g., at most 5%, at most 10%, at most 15% at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, or at most 95% from the constitutive or baseline activity. In still other aspects of this embodiment, an inverse estrogen antagonist reduces a constitutive or baseline ER activity by, e.g., about 5% to about 100%, about 10% to about 100%, about 15% to about 100%, about 20% to about 100%, about 25% to about 100%, about 30% to about 100%, about 35% to about 100%, about 40% to about 100%, about 45% to about 100%, or about 50% to about 100% from the constitutive or baseline activity.

In another embodiment, a compound capable of modulating activity of a hormone is a selective estrogen receptor modulator (SERM). A SERM differs in chemical structure from the endogenous hormone estrogen, but nevertheless bind to the same ER. A SERM has the ability to promote ER interactions with different proteins such as transcriptional coactivator or corepressors. Furthermore, the ratio of coactivator to corepressor varies in different tissues. As a consequence, the same SERM may be an agonist in some tissue (where coactivators predominate) while antagonistic in other tissues (where corepressors dominate). SERMs preferentially bind to either the α-subtype or the β-subtype of the ER. In addition, the different ER combinations may respond differently to various SERMs, which may translate into tissue selective agonistic and antagonistic effects. Tamoxifen, for example, is an antagonist in breast and is, therefore, used as a breast cancer treatment but an ER agonist in bone (thereby preventing osteoporosis) and a partial agonist in the endometrium (increasing the risk of uterine cancer).

In aspects of this embodiment, a SERM blocks an ER activity that results in a biological response that is, e.g., about 5%, about 10%, about 15% about 20%, about 25%, about 30%, about 35%, about 40%, about 45%, about 50%, about 55%, about 60%, about 65%, about 70%, about 75%, about 80%, about 85%, about 90%, about 95%, or about 100% of a maximal biological response elicited by estrogen. In other aspects of this embodiment, a SERM blocks an ER activity that results in a biological response that is, e.g., at least 5%, at least 10%, at least 15% at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of a maximal biological response elicited by estrogen. In yet other aspects of this embodiment, a SERM blocks an ER activity that results in a biological response that is, e.g., at most 5%, at most 10%, at most 15% at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, or at most 95% of a maximal biological response elicited by estrogen. In still other aspects of this embodiment, a SERM blocks an ER activity that results in a biological response that is, e.g., about 5% to about 100%, about 10% to about 100%, about 15% to about 100%, about 20% to about 100%, about 25% to about 100%, about 30% to about 100%, about 35% to about 100%, about 40% to about 100%, about 45% to about 100%, or about 50% to about 100% of a maximal biological response elicited by estrogen. Examples of suitable SERMs include, without limitation, 2-Hydroxyestrone, Acolbifene, Afimoxifene, Arzoxifene, Bazedoxifene, Clomifene, Clomifenoxide, Cyclofenil, Droloxifene, Enclomifene, Endoxifen, Epimestrol, Femarelle, Fispemifene, GW5638, Idoxifene, Lasofoxifene, Levormeloxifene, Menerba, Mepitiostane, Miproxifene, Nafoxidine, Nitromifene, Ormeloxifene, Ospemifene, Panomifene, Pipendoxifene, Prinaberel, Raloxifene, Resveratrol, SS1010, Sivifene, Tamoxifen, TAS-108, Tesmilifene, Tibolone, Toremifene, Trioxifene, Y-134, Zindoxifene, and Zuclomifene.

In another embodiment, a compound capable of modulating activity of a hormone is an estrogen biosynthesis enzyme inhibitor. In females, synthesis of estrogens starts in theca interna cells in the ovary, by the synthesis of androstenedione from cholesterol. Androstenedione is a substance of moderate androgenic activity. This compound crosses the basal membrane into the surrounding granulosa cells, where it is converted to estrone or estradiol, either immediately or through testosterone. The conversion of testosterone to estradiol, and of androstenedione to estrone, is catalyzed by the enzyme aromatase.

Aromatase, also called estrogen synthetase or estrogen synthase, is an enzyme responsible for a key step in the biosynthesis of estrogens. It is a member of the cytochrome P450 superfamily (EC 1.14.14.1), which are monooxygenases that catalyze many reactions involved in steroidogenesis. In particular, aromatase is responsible for the aromatization of androgens into estrogens. The aromatase enzyme can be found in many tissues including gonads, brain, adipose tissue, placenta, blood vessels, skin, bone, and endometrium, as well as in tissue of endometriosis, uterine fibroids, breast cancer, and endometrial cancer. Aromatase inhibitors prevent the conversion of testosterone into estrogen, thereby diverting testosterone into the biosynthetic pathway of progesterone. Thus, inhibition of aromatase leads to profound hypoestrogenism (low estrogen levels). An aromatase inhibitor may be an irreversible inhibitor that permanent deactivates aromatase or reversible inhibitor that competes with aromatase for the substrate binding. In addition, an aromatase inhibitor may be selective or non-selective. Examples of suitable aromatase inhibitors include, without limitation, 1,4,6-Androstatriene-3,17-dione (ATD), 4-Androstene-3,6,17-trione (6-OXO), 4-Cyclohexylaniline, 4-Hydroxyandrostenedione, 4-Hydroxytestosterone, 5α-DHNET, Abyssinone II, Aminoglutethimide, Anastrozole, Ascorbic acid (Vitamin C), Atamestane, Bifonazole, CGP-45,688, CGS-47,645, Clotrimazole, DHT, Difeconazole, Econazole, Exemestane, Fadrozole, Fenarimol, Finrozole, Formestane, Imazalil, Isoconazole, Ketoconazole, Letrozole, Liarozole, MEN-11066, Miconazole, Minamestane, Nimorazole, NKS01, Norethindrone, ORG-33,201, Penconazole, Plomestane, Prochloraz, Propioconazole, Pyridoglutethimide, Rog letimide, Rotenone, Talarozole, Testolactone, Tioconazole, Triadimefon, Triadimenol, Troglitazone, Vorozole, YM511, and Zinc.

CYP17A1 (also known as cytochrome P450 17A1, or steroid 17-alpha-monooxygenase, or 17α-hydroxylase/17,20 lyase/17,20 desmolase) is a key enzyme in the steroidogenic pathway that produces progestins, mineralocorticoids, glucocorticoids, androgens, and estrogens. CYP17A1 is a cytochrome P450 enzyme that acts upon pregnenolone and progesterone to add a hydroxyl (—OH) group at carbon 17 of the steroid D ring (the 17alpha-hydroxylase activity), or acts upon 17-hydroxyprogesterone and 17-hydroxypregnenolone to split the side-chain off the steroid nucleus (the 17,20-lyase activity). CYP17A1 also converts cholesterol to pregnenolone via its 20,22-desmolase activity. Examples of suitable CYP17A1 inhibitors include, without limitation, a 17α-Hydroxylase inhibitor, a 17,20-Lyase inhibitor, and a 20,22-Desmolase inhibitor. Examples of suitable 17α-Hydroxylase inhibitors include, without limitation, 22-ABC, 22-Oxime, Abiraterone, Bifonazole, Clotrimazole, Cyanoketone, Cyproterone, Danazol, Desogestrel, Econazole, Galeterone, Gestrinone, Isoconazole, Ketoconazole, L-39, Levonorgestrel, and Liarozole. Examples of suitable 17,20-Lyase inhibitors include, without limitation, 22-ABC, 22-Oxime, Abiraterone, Bifonazole, Clotrimazole, Cyanoketone, Cyproterone, Danazol, Desogestrel, Econazole, Galeterone, Gestrinone, Isoconazole, Ketoconazole, L-39, Levonorgestrel, Liarozole, LY-207,320, MDL-27,302, Miconazole, Mifepristone, Norethisterone, Orteronel, Pioglitazone, Rosiglitazone, Spironolactone, Stanozolol, SU-10,603, Tioconazole, TGF-β, Troglitazone, VN/87-1, and YM116. Examples of suitable 20,22-Desmolase inhibitors include, without limitation, 22-ABC, 3,3′-Dimethoxybenzidine, 3-Methoxybenzidine, Aminoglutethimide, Cyanoketone, Danazol, Etomidate, Mitotane, and Trilostane.

3-β-HSD (or 3-β-hydroxysteroid dehydrogenase/Δ-5-4 isomerase) is an enzyme complex that catalyses the synthesis of progesterone from pregnenolone, 17-hydroxyprogesterone from 17-hydroxypregnenolone, and androstenedione from dehydroepiandrosterone (DHEA), and testosterone from androstenediol in the adrenal gland. It is the only enzyme in the adrenal pathway of corticosteroid synthesis that is not a member of the Cytochrome P450 family. In humans, there are two 3-β-HSD isozymes encoded by the HSD3B1 and HSD3B2 genes, respectively. It is also known as delta 5-delta 4-isomerase, which catalyzes the oxidative conversion of delta 5-3 beta-hydroxysteroids to the delta 4-3-keto configuration and is, therefore, essential for the biosynthesis of all classes of hormonal steroids, namely progesterone, glucocorticoids, mineralocorticoids, androgens, and estrogens. Examples of suitable 3β-HSD inhibitors include, without limitation, 4-MA, Azastene, Cyanoketone, Danazol, Desogestrel, Epostane, Genistein, Gestrinone, Levonorgestrel, Medroxyprogesterone, Medroxyprogesterone acetate, Metyrapone, Norethisterone, Oxymetholone, Pioglitazone, Rosiglitazone, Trilostane, and Troglitazone.

17β-Hydroxysteroid dehydrogenases (17β-HSD), also known as 17-ketosteroid oxidoreductases or simply as 17-ketosteroid reductases (17-KSR), are a group of alcohol oxidoreductases which catalyse the dehydrogenation of 17-hydroxysteroids in steroidogenesis. This includes interconversion of DHEA and androstenediol, androstenedione and testosterone, and estrone and estradiol, respectively. Examples of suitable 17β-HSD inhibitors include, without limitation, Danazol and Simvastatin.

In another embodiment, a compound capable of modulating activity of a hormone is an estrogen agonist. Estrogen agonists are a group of naturally-occurring hormones or chemically-synthesized compound that stimulates or controls the development and maintenance of female characteristics in vertebrates by binding to an estrogen receptor. This includes the activity of the accessory female sex organs and development of female secondary sex characteristics. Androgens are also the original anabolic steroids and the precursor of all estrogens, the female sex hormones. Characterized by its basic 18-carbon skeleton, called an estrane skeleton, an estrogen agonist, includes both endogenous and exogenous naturally occurring compounds. Synthetic estrogen agonist can be based upon an estrane skeleton or structures not derived from this skeleton. The primary and most well-known naturally occurring estrogen agonists are estrone (E1), estradiol (E2), estriol (E3), and estetrol (E4)

Like an estrogen antagonists disclosed herein, an estrogen agonist mediates its effects through an estrogen receptor (ER). Estrogen agonist binding is necessary to induce ER activity. Examples of suitable estrogen agonists include, without limitation, 5α-Androstane-3β,17β-diol, 16α-Hydroxyestrone, Alestramustine, Almestrone, Benzestrol, Bifluranol, Broparestrol, Carbestrol, Chlorotrianisene, Cloxestradiol, Daidzein, DHEA, Dienestrol, Diethylstilbestrol, Diethylstilbestrol diphosphate, Diosgenin, Doisynoestrol, DPN, Epiestriol, Epimestrol, Eptamestrol/Etamestrol, ERB-041, Equilenin, Equilin, Estetrol, Estradiol, Estradiol benzoate, Estradiol butyrylacetate, Estradiol cypionate, Estradiol dienanthate, Estradiol dipropionate, Estradiol diundecylate, Estradiol diundecylenate, Estradiol enanthate, Estradiol furoate, Estradiol hemihydrate, Estradiol hemisuccinate, Estradiol hexahydrobenzoate, Estradiol monopropionate, Estradiol palmitate, Estradiol pivalate, Estradiol propoxyphenylpropionate, Estradiol stearate, Estradiol succinate, Estradiol undecylate, Estradiol valerate, Estramustine, Estramustine phosphate, Estrapronicate, Estrazinol, Estriol, Estriol succinate, Estriol tripropionate, Estrofurate, Estromustine, Estrone, Estrone acetate, Estrone cyanate, Estrone sulfate, Estrone tetraacetylglucoside, Estropipate, Etamestrol, Ethinylestradiol, Ethinylestradiol 3-isopropylsulfonate, Etynodiol diacetate, FERb 033, Fenestrel, Fosfestrol, Furostilbestrol, Hexestrol, Hexestrol diacetate, Hexestrol dicaprylate, Hexestrol diphosphate, Hexestrol dipropionate, Hydroxyestrone diacetate, Liquiritigenin, Mestranol, Methallenestril, Methestrol, Methestrol dipropionate, Moxestrol, Nilestriol, Orestrate, Polyestradiol phosphate, PPT, Promestriene, Promethoestrol, Quinestradol, Quinestrol, β-Sitosterol, and WAY-199,070.

An estrogen agonist may be a full agonist or a partial agonist. A full estrogen agonist is able to activate an ER and result in a maximal biological response. The natural endogenous ligand with the greatest efficacy for a given receptor is by definition a full agonist (100% efficacy). In aspects of this embodiment, a full estrogen agonist activates an ER and results in a biological response that is, e.g., about 90%, about 91%, about 92%, about 93%, about 94%, about 95%, about 96%, about 97%, about 98%, about 99%, or about 100% of a maximal biological response elicited by estrogen. In other aspects of this embodiment, a full estrogen agonist activates an ER and results in a biological response that is, e.g., at least 90%, at least 91%, at least 92%, at least 93%, at least 94%, at least 95%, at least 96%, at least 97%, at least 98%, or at least 99% of a maximal biological response elicited by estrogen. In yet other aspects of this embodiment, a full estrogen agonist activates an ER and results in a biological response that is between, e.g., about 90% to about 98%, about 91% to about 98%, about 92% to about 98%, about 93% to about 98%, about 94% to about 98%, about 95% to about 98%, about 90% to about 100%, about 91% to about 100%, about 92% to about 100%, about 93% to about 100%, about 94% to about 100%, or about 95% to about 100% of a maximal biological response elicited by estrogen.

Although activating an ER, a partial estrogen agonist does not fully activate the receptor, causing responses which are partial compared to a full agonist. In aspects of this embodiment, a partial estrogen agonist activates an ER and results in a biological response that is, e.g., about 40%, about 45% about 50%, about 55%, about 60%, about 65%, about 70%, about 75%, about 80%, about 85%, or about 90% of a maximal biological response elicited by estrogen. In other aspects of this embodiment, a partial estrogen agonist activates an ER and results in a biological response that is, e.g., at least 40%, at least 45% at least 50%, at least 55%, at least 60%, at least 65%, at least 70%, at least 75%, at least 80%, at least 85%, or at least 90% of a maximal biological response elicited by estrogen. In yet other aspects of this embodiment, a partial estrogen agonist activates an ER and results in a biological response that is, e.g., at most 40%, at most 45% at most 50%, at most 55%, at most 60%, at most 65%, at most 70%, at most 75%, at most 80%, at most 85%, or at most 90% of a maximal biological response elicited by estrogen. In still other aspects of this embodiment, a partial estrogen agonist activates an ER and results in a biological response that is between, e.g., about 40% to about 85%, about 45% to about 85%, about 50% to about 85%, about 55% to about 85%, about 60% to about 85%, about 65% to about 85%, about 70% to about 85%, about 75% to about 85%, about 80% to about 85%, about 40% to about 90%, about 45% to about 90%, about 50% to about 90%, about 55% to about 90%, about 60% to about 90%, about 65% to about 90%, about 70% to about 90%, about 75% to about 90%, about 80% to about 90%, or about 85% to about 90% of a maximal biological response elicited by estrogen.

In another embodiment, a compound capable of modulating activity of a hormone is an androgen agonist. Androgen agonists are a group of naturally-occurring hormones or chemically-synthesized compound that stimulates or controls the development and maintenance of male characteristics in vertebrates by binding to an androgen receptor. This includes the activity of the accessory male sex organs and development of male secondary sex characteristics. Androgens are also the original anabolic steroids and the precursor of all estrogens, the female sex hormones. Characterized by its basic 19-carbon skeleton, called an andrane skeleton, an androgen agonist, includes both endogenous and exogenous naturally occurring compounds. Synthetic androgen agonist can be based upon an andrane skeleton or structures not derived from this skeleton. The primary and most well-known naturally occurring androgen agonist is Testosterone, other less important naturally occurring androgen are Dihydrotestosterone (DHT), Dehydroepiandrosterone (DHEA), Androsterone, Androstenediol, and Androstenedione.

An androgen agonist mediates its effects through an androgen receptor (AR, also known as NR3C4 or nuclear receptor subfamily 3, group C, member 4), a nuclear receptor found inside cells. In humans, the androgen receptor is encoded by the AR gene located on the X chromosome at Xq11-12, and it produces two main forms, A and B, that differ in their molecular weight. In common with other steroid receptors, an AR comprises a N-terminal NB domain is able to transactivate gene transcription in the absence of bound ligand. While this region is able to activate gene transcription without ligand, this activation is weak and more selective compared to the activation provided by the E domain. The C domain, also known as the DNA-binding domain, includes a highly-conserved zinc-finger that binds to target DNA sequences called androgen-responsive elements (ARE). The D domain is a hinge region that connects the C and E domains. The E domain contains the ligand binding cavity as well as binding sites for coactivator and corepressor proteins. The E-domain in the presence of bound ligand is able to activate gene transcription. The C-terminal F domain function is not entirely clear and is variable in length. ARs function as dimeric molecules in nuclei to regulate the transcription of target genes in a ligand-responsive manner.

Androgen agonist binding is necessary to induce AR activity. In the absence of androgen agonist binding, the carboxyl terminal end of AR inhibits transcription, even though this receptor weakly associated with nuclear components. After androgen agonist binding, AR restructuring with dimerization follows that 1) induces a structural change that removes the inhibitory action; 2) greatly increases receptor affinity for ARE; and 3) the complex enters the nucleus and specifically binds to DNA at the AREs. There transcription takes place, resulting in formation of messenger RNA that is translated by ribosomes to produce specific proteins. Examples of suitable androgen agonists include, without limitation, 1-Androstenediol, 1-Testosterone, 1,4-Androstenedione, 4-Androstenediol, 4-Chlordehydromethyltestosterone, 4-Hydroxytestosterone, 5-Androstenediol, 5α-Androst-1-ene-3,17-dione, 11-Ketotestosterone, 17-((1-Oxoheptyl)oxy)androstan-3-one, 17-((1-Oxopentyl)oxy)androstan-3-one, 17-(1-Oxopropoxy)androstan-3-one, 17β-Hydroxyandrost-2-ene-2-carbonitrile, 19-Norandrostenediol, 19-Norandrostenedione, 19-Norandrosterone, 19-Nordehydrotestosterone, Adrenosterone, Androisoxazole, Androstanolone, Androstenediol, Androstenedione, Androsterone, Bolandiol, Bolasterone, Bolazine, Boldenone, Boldione, Bolenol, Bolmantalate, Calusterone, Clostebol, Cloxotestosterone, Danazol, DHEA, Dehydroepiandrosterone sulfate (DHEA-S), Desogestrel, Desoxymethyltestosterone, DHT, Drostanolone, Enestebol, Epitiostanol, Ethisterone, Ethyldienolone, Ethylestrenol, Fluoxymesterone, Formebolone, Furazabol, Hexabolan, Human Growth Hormone (HGH), Hydroxystenozole, Levonorgestrel, Mebolazine, Mepitiostane, Mesabolone, Mestanolone, Mesterolone, Metandienone, Methandrostenolone, Metenolone, Metenolone acetate, Metenolone enanthate, Methandriol, Methandriol propionate, Methasterone, Methylestrenolone, Methyltestosterone, Metribolone, Mibolerone, Nandrolone, Nandrolone caproate, Nandrolone cypionate, Nandrolone cyclohexane carboxylate, Nandrolone cyclohexylpropionate, Nandrolone cyclotate, Nandrolone decanoate, Nandrolone furylpropionate, Nandrolone hexyloxyphenylpropionate, Nandrolone hydrogen succinate, Nandrolone laurate, Nandrolone phenylpropionate, Nandrolone propionate, Nandrolone undecyclate, Norbolethone, Norclostebol, Norethandrolone, Norethisterone, Norgestrel, Oxabolone, Oxabolone cypionate, Oxandrolone, Oxendolone, Oxymesterone, Oxymetholone, Penmesterol, Propetandrol, Quinbolone, Roxibolone, Silandrone, Stanozolol, Stenbolone, Stenbolone acetate, Testolactone, Testosterone, Testosterone enanthate, Testosterone ketolaurate, Testosterone nicotinate, Testosterone propionate, Testosterone undecanoate, THG, Thiomesterone, Tibolone, Tiomesterone, Trenbolone, Trenbolone acetate, and Trestolone.

An androgen agonist may be a full agonist or a partial agonist. A full androgen agonist is able to activate an AR and result in a maximal biological response. The natural endogenous ligand with the greatest efficacy for a given receptor is by definition a full agonist (100% efficacy). In aspects of this embodiment, a full androgen agonist activates an AR and results in a biological response that is, e.g., about 90%, about 91%, about 92%, about 93%, about 94%, about 95%, about 96%, about 97%, about 98%, about 99%, or about 100% of a maximal biological response elicited by testosterone. In other aspects of this embodiment, a full androgen agonist activates an AR and results in a biological response that is, e.g., at least 90%, at least 91%, at least 92%, at least 93%, at least 94%, at least 95%, at least 96%, at least 97%, at least 98%, or at least 99% of a maximal biological response elicited by testosterone. In yet other aspects of this embodiment, a full androgen agonist activates an AR and results in a biological response that is between, e.g., about 90% to about 98%, about 91% to about 98%, about 92% to about 98%, about 93% to about 98%, about 94% to about 98%, about 95% to about 98%, about 90% to about 100%, about 91% to about 100%, about 92% to about 100%, about 93% to about 100%, about 94% to about 100%, or about 95% to about 100% of a maximal biological response elicited by testosterone.

Although activating an AR, a partial androgen agonist does not fully activate the receptor, causing responses which are partial compared to a full agonist. In aspects of this embodiment, a partial androgen agonist activates an AR and results in a biological response that is, e.g., about 40%, about 45% about 50%, about 55%, about 60%, about 65%, about 70%, about 75%, about 80%, about 85%, or about 90% of a maximal biological response elicited by testosterone. In other aspects of this embodiment, a partial androgen agonist activates an AR and results in a biological response that is, e.g., at least 40%, at least 45% at least 50%, at least 55%, at least 60%, at least 65%, at least 70%, at least 75%, at least 80%, at least 85%, or at least 90% of a maximal biological response elicited by testosterone. In yet other aspects of this embodiment, a partial androgen agonist activates an AR and results in a biological response that is, e.g., at most 40%, at most 45% at most 50%, at most 55%, at most 60%, at most 65%, at most 70%, at most 75%, at most 80%, at most 85%, or at most 90% of a maximal biological response elicited by testosterone. In still other aspects of this embodiment, a partial androgen agonist activates an AR and results in a biological response that is between, e.g., about 40% to about 85%, about 45% to about 85%, about 50% to about 85%, about 55% to about 85%, about 60% to about 85%, about 65% to about 85%, about 70% to about 85%, about 75% to about 85%, about 80% to about 85%, about 40% to about 90%, about 45% to about 90%, about 50% to about 90%, about 55% to about 90%, about 60% to about 90%, about 65% to about 90%, about 70% to about 90%, about 75% to about 90%, about 80% to about 90%, or about 85% to about 90% of a maximal biological response elicited by testosterone.

In another embodiment, a compound capable of modulating activity of a hormone is a selective androgen receptor modulator (SARM). A SARM differs in chemical structure from the endogenous hormone progesterone, but nevertheless bind to the same AR. A SARM has the ability to promote AR interactions with different proteins such as transcriptional coactivator or corepressors. Furthermore, the ratio of coactivator to corepressor varies in different tissues. As a consequence, the same SARM may be an agonist in some tissue (where coactivators predominate) while antagonistic in other tissues (where corepressors dominate). Examples of suitable SARMs include, without limitation, AC-262,356, Andarine, BMS-564,929, Enobosarm, LGD-2226, LGD-3303, S-23, and S-40503.

In aspects of this embodiment, a SARM activates an AR and results in a biological response that is, e.g., about 5%, about 10%, about 15% about 20%, about 25%, about 30%, about 35%, about 40%, about 45%, about 50%, about 55%, about 60%, about 65%, about 70%, about 75%, about 80%, about 85%, about 90%, about 95%, or about 100% of a maximal biological response elicited by testosterone. In other aspects of this embodiment, a SARM activates an AR and results in a biological response that is, e.g., at least 5%, at least 10%, at least 15% at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of a maximal biological response elicited by testosterone. In yet other aspects of this embodiment, a SARM activates an AR and results in a biological response that is, e.g., at most 5%, at most 10%, at most 15% at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, or at most 95% of a maximal biological response elicited by testosterone. In still other aspects of this embodiment, a SARM activates an AR and results in a biological response that is, e.g., about 5% to about 100%, about 10% to about 100%, about 15% to about 100%, about 20% to about 100%, about 25% to about 100%, about 30% to about 100%, about 35% to about 100%, about 40% to about 100%, about 45% to about 100%, or about 50% to about 100% of a maximal biological response elicited by testosterone.

In another embodiment, a compound capable of modulating activity of a hormone is an androgen antagonist. Androgen antagonists are a group of naturally-occurring hormones or chemically-synthesized compound that suppress or controls the development and maintenance of male characteristics in vertebrates by binding to an androgen receptor. This includes the activity of the accessory male sex organs and development of male secondary sex characteristics. Characterized by its basic 19-carbon skeleton, called an andrane skeleton, an androgen antagonist, includes both endogenous and exogenous naturally occurring compounds. Synthetic androgen antagonist can be based upon an andrane skeleton or structures not derived from this skeleton.

Like an androgen agonists disclosed herein, an androgen antagonist mediates its effects through an androgen receptor (AR). Androgen antagonist binding is necessary to supress AR activity. Examples of suitable androgen antagonist include, without limitation, ARN-509, Benorterone, Bicalutamide, BMS-641,988, BOMT, Canrenoic acid, Canrenone, Chlormadinone acetate, Cimetidine, Cioteronel, Cyproterone, Cyproterone acetate, Delanterone, Dienogeste, Enzalutamide, Epitestosterone, Flutamide, Galeterone, Hydroxyflutamide, Hydroxyprogesterone caproate, Inocoterone, Ketoconazole, Megestrol acetate, Metogest, Mifepristone, Nilutamide, Nomegestrol, Nordinone, Norgestimate, Osaterone, Oxendolone, Potassium canrenoate, Progesterone, R2956, Rosterolone, RU-58642, RU-58841, Spironolactone, Topterone, and Zanoterone.

An androgen antagonist mediates its effects through an AR and may be a full antagonist, a partial antagonist, or an inverse agonist. A full androgen antagonist binds to an AR, but does not activate the nuclear hormone receptor. This results in receptor blockage, inhibiting the binding of androgen agonists, androgen partial agonist, and/or androgen inverse agonists.

In aspects of this embodiment, a full androgen antagonist blocks an AR activity that results in a biological response that is, e.g., about 0%, about 1%, about 2%, about 3%, about 4%, about 5%, about 6%, about 7%, about 8%, about 9%, or about 10% of a maximal biological response elicited by androgen. In other aspects of this embodiment, a full androgen antagonist blocks an AR activity that results in a biological response that is, e.g., at most 1%, at most 2%, at most 3%, at most 4%, at most 5%, at most 6%, at most 7%, at most 8%, at most 9%, or at most 10% of a maximal biological response elicited by androgen. In yet other aspects of this embodiment, a full androgen antagonist blocks an AR activity that results in a biological response that is between, e.g., about 0% to about 5%, about 0% to about 6%, about 0% to about 7%, about 0% to about 8%, about 0% to about 9%, about 0% to about 10%, about 1% to about 5%, about 1% to about 6%, about 1% to about 7%, about 1% to about 8%, about 1% to about 9%, about 1% to about 10%, about 2% to about 5%, about 2% to about 6%, about 2% to about 7%, about 2% to about 8%, about 2% to about 9%, or about 2% to about 10% of a maximal biological response elicited by androgen.

Although blocking an AR, a partial androgen antagonist does not fully block receptor activity, causing responses which are partial compared to a full agonist. In aspects of this embodiment, a partial androgen antagonist blocks an AR activity that results in a biological response that is, e.g., about 10%, about 15% about 20%, about 25%, about 30%, about 35%, or about 40% of a maximal biological response elicited by androgen. In other aspects of this embodiment, a partial androgen antagonist blocks an AR activity that results in a biological response that is, e.g., at most 10%, at most 15% at most 20%, at most 25%, at most 30%, at most 35%, or at most 40% of a maximal biological response elicited by androgen. In yet other aspects of this embodiment, a partial androgen antagonist blocks an AR activity that results in a biological response that is between, e.g., about 10% to about 20%, about 10% to about 25%, about 10% to about 30%, about 10% to about 35%, about 10% to about 40%, about 15% to about 20%, about 15% to about 25%, about 15% to about 30%, about 15% to about 35%, about 15% to about 40%, about 20% to about 25%, about 20% to about 30%, about 20% to about 35%, or about 20% to about 40% of a maximal biological response elicited by androgen.

A pharmaceutical composition disclosed herein may optionally include a pharmaceutically-acceptable carrier that facilitates processing of an active ingredient into pharmaceutically-acceptable compositions. As used herein, the term “pharmacologically-acceptable carrier” is synonymous with “pharmacological carrier” and means any carrier that has substantially no long term or permanent detrimental effect when administered and encompasses terms such as “pharmacologically acceptable vehicle, stabilizer, diluent, additive, auxiliary or excipient.” Such a carrier generally is mixed with an active compound or permitted to dilute or enclose the active compound and can be a solid, semi-solid, or liquid agent. It is understood that the active ingredients can be soluble or can be delivered as a suspension in the desired carrier or diluent. Any of a variety of pharmaceutically acceptable carriers can be used including, without limitation, aqueous media such as, e.g., water, saline, glycine, hyaluronic acid and the like; solid carriers such as, e.g., mannitol, lactose, starch, magnesium stearate, sodium saccharin, talcum, cellulose, glucose, sucrose, magnesium carbonate, and the like; solvents; dispersion media; coatings; antibacterial and antifungal agents; isotonic and absorption delaying agents; or any other inactive ingredient. Selection of a pharmacologically acceptable carrier can depend on the mode of administration. Except insofar as any pharmacologically acceptable carrier is incompatible with the active ingredient, its use in pharmaceutically acceptable compositions is contemplated. Non-limiting examples of specific uses of such pharmaceutical carriers can be found in Pharmaceutical Dosage Forms and Drug Delivery Systems (Howard C. Ansel et al., eds., Lippincott Williams & Wilkins Publishers, 7th ed. 1999); REMINGTON: THE SCIENCE AND PRACTICE OF PHARMACY (Alfonso R. Gennaro ed., Lippincott, Williams & Wilkins, 20th ed. 2000); Goodman & Gilman's The Pharmacological Basis of Therapeutics (Joel G. Hardman et al., eds., McGraw-Hill Professional, 10th ed. 2001); and Handbook of Pharmaceutical Excipients (Raymond C. Rowe et al., APhA Publications, 4th edition 2003). These protocols are routine procedures and any modifications are well within the scope of one skilled in the art and from the teaching herein.

A pharmaceutical composition disclosed herein can optionally include, without limitation, other pharmaceutically acceptable components (or pharmaceutical components), including, without limitation, buffers, preservatives, tonicity adjusters, salts, antioxidants, osmolality adjusting agents, physiological substances, pharmacological substances, bulking agents, emulsifying agents, wetting agents, flavoring agents, coloring agents, and the like. Various buffers and means for adjusting pH can be used to prepare a pharmaceutical composition disclosed herein, provided that the resulting preparation is pharmaceutically acceptable. Such buffers include, without limitation, acetate buffers, citrate buffers, phosphate buffers, neutral buffered saline, phosphate buffered saline and borate buffers. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole and butylated hydroxytoluene. Useful preservatives include, without limitation, benzalkonium chloride, chlorobutanol, thimerosal, phenylmercuric acetate, phenylmercuric nitrate, a stabilized oxy chloro composition and chelants, such as, e.g., DTPA or DTPA-bisamide, calcium DTPA, and CaNaDTPA-bisamide. Tonicity adjustors useful in a pharmaceutical composition include, without limitation, salts such as, e.g., sodium chloride, potassium chloride, mannitol or glycerin and other pharmaceutically acceptable tonicity adjustor. The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. It is understood that these and other substances known in the art of pharmacology can be included in a pharmaceutical composition.

A therapeutic compound disclosed herein, or a pharmaceutical composition disclosed herein, may be formulated for either local or systemic delivery using topical, enteral or parenteral routes of administration. Additionally, a therapeutic compound disclosed herein may be formulated by itself in a pharmaceutical composition, or may be formulated together with one or more other therapeutic compounds disclosed herein in a single pharmaceutical composition. In one embodiment, a compound or a pharmaceutical composition disclosed herein is used in the manufacture of a medicament.

A therapeutic compound disclosed herein, or a pharmaceutical composition disclosed herein, may be made into an inhaled formulation. Inhaled formulations suitable for enteral or parenteral administration include, without limitation, aerosols, dry powders. A therapeutic compound or composition disclosed herein intended for such administration may be prepared according to any method known to the art for the manufacture of pharmaceutical compositions. In such inhaled dosage forms, the therapeutic compound may be prepared for delivery as an aerosol in a liquid propellant for use in a pressurised (PDI) or other metered dose inhaler (MDI). Propellants suitable for use in a PDI or MDI include, without limitation, CFC-12, HFA-134a, HFA-227, HCFC-22 (difluorochloromethane), HFA-152 (difluoroethane and isobutane). A therapeutic compound may also be delivered using a nebulisers or other aerosol delivery system. A therapeutic compound may be prepared for delivery as a dry powder for use in a dry powder inhaler (DPI). A dry powder for use in the inhalers will usually have a mass median aerodynamic diameter of less than 30 pm, preferably less than 20 pm and more preferably less than 10 pm. Microparticles having aerodynamic diameters in the range of about 5 pm to about 0.5 pm will generally be deposited in the respiratory bronchioles, whereas smaller particles, having aerodynamic diameters in the range of about 2 pm to about 0.05 pm, are likely to be deposited in the alveoli. A DPI may be a passive delivery mechanism, which relies on the individual's inspiration to introduce the particles into the lungs, or an active delivery mechanism, requiring a mechanism for delivering the powder to the individual. In inhalatory formulations, a therapeutically effective amount of a therapeutic compound disclosed herein for an inhaled formulation may be between about 0.0001% (w/v) to about 60% (w/v), about 0.001% (w/v) to about 40.0% (w/v), or about 0.01% (w/v) to about 20.0% (w/v). In inhalatory formulations, a therapeutically effective amount of a therapeutic compound disclosed herein for an inhaled formulation may also be between about 0.0001% (w/w) to about 60% (w/w), about 0.001% (w/w) to about 40.0% (w/w), or about 0.01% (w/w) to about 20.0% (w/w).

A therapeutic compound disclosed herein, or a pharmaceutical composition disclosed herein, may be made into a solid formulation. Solid formulations suitable for enteral or parenteral administration include, without limitation, capsules, tablets, pills, troches, lozenges, powders and granules suitable for inhalation or for reconstitution into sterile injectable solutions or dispersions. A therapeutic compound or composition disclosed herein intended for such administration may be prepared according to any method known to the art for the manufacture of pharmaceutical compositions. In such solid dosage forms, the therapeutic compound may be admixed with (a) at least one inert customary excipient (or carrier), such as, e.g., sodium citrate or dicalcium phosphate or (b) fillers or extenders, as for example, starch, lactose, sucrose, glucose, mannitol, isomalt, and silicic acid, (c) binders, such as, e.g., carboxymethylcellulose, alignates, gelatin, polyvinylpyrrolidone, sucrose and acacia, (d) humectants, such as, e.g., glycerol, (e) disintegrating agents, such as, e.g., agar-agar, calcium carbonate, corn starch, potato starch, tapioca starch, alginic acid, certain complex silicates and sodium carbonate, (f) solution retarders, such as, e.g., paraffin, (g) absorption accelerators, such as, e.g., quaternary ammonium compounds, (h) wetting agents, such as, e.g., cetyl alcohol and glycerol monostearate, (i) adsorbents, such as, e.g., kaolin and bentonite, (j) lubricants, such as, e.g., talc, stearic acid, calcium stearate, magnesium stearate, solid polyethylene glycols, sodium lauryl sulfate or mixtures thereof, and (k) buffering agents. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. In solid formulations, a therapeutically effective amount of a therapeutic compound disclosed herein typically may be between about 0.0001% (w/w) to about 60% (w/w), about 0.001% (w/w) to about 40.0% (w/w), or about 0.01% (w/w) to about 20.0% (w/w).

A therapeutic compound disclosed herein, or a pharmaceutical composition disclosed herein, may be made into a semi-solid formulation. Semi-solid formulations suitable for topical administration include, without limitation, ointments, creams, salves, and gels. A therapeutic compound or composition disclosed herein intended for such administration may be prepared according to any method known to the art for the manufacture of pharmaceutical compositions. In semi-solid formulations, a therapeutically effective amount of a therapeutic compound disclosed herein typically may be between about 0.0001% (w/v) to about 60% (w/v), about 0.001% (w/v) to about 40.0% (w/v), or about 0.01% (w/v) to about 20.0% (w/v). In semi-solid formulations, a therapeutically effective amount of a therapeutic compound disclosed herein typically may also be between about 0.0001% (w/w) to about 60% (w/w), about 0.001% (w/w) to about 40.0% (w/w), or about 0.01% (w/w) to about 20.0% (w/w).

A therapeutic compound disclosed herein, or a pharmaceutical composition disclosed herein, may be made into a liquid formulation. Liquid formulations suitable for enteral or parenteral administration include, without limitation, solutions, syrups, elixirs, dispersions, emulsions, and suspensions. A therapeutic compound or composition disclosed herein intended for such administration may be prepared according to any method known to the art for the manufacture of pharmaceutical compositions. In such liquid dosage forms, a therapeutic compound or composition disclosed herein may be admixed with (a) suitable aqueous and nonaqueous carriers, (b) diluents, (c) solvents, such as, e.g., water, ethanol, propylene glycol, polyethyleneglycol, glycerol, vegetable oils, such as, e.g., rapeseed oil and olive oil, and injectable organic esters such as ethyl oleate; and/or fluidity agents, such as, e.g., surfactants or coating agents like lecithin. In the case of dispersions and suspensions, fluidity can also be controlled by maintaining a particular particle size. In liquid formulations, a therapeutically effective amount of a therapeutic compound disclosed herein typically may be between about 0.0001% (w/v) to about 60% (w/v), about 0.001% (w/v) to about 40.0% (w/v), or about 0.01% (w/v) to about 20.0% (w/v).

Syrups and elixirs may be formulated with sweetening agents, for example glycerol, propylene glycol, sorbitol or sucrose. Such formulations may also contain a demulcent, a preservative, flavoring agents, and coloring agents.

Liquid suspensions may be formulated by suspending a therapeutic compound disclosed herein in admixture with excipients suitable for the manufacture of aqueous suspensions. Such excipients are suspending agents, for example sodium carboxymethylcellulose, methylcellulose, hydroxypropylmethylcellulose, sodium alginate, pectin, polyvinyl pyrrolidone, polyvinyl alcohol, natural gum, agar, gum tragacanth and gum acacia; dispersing or wetting agents may be a naturally occurring phosphatide, for example lecithin, or condensation products of an alkylene oxide with fatty acids, for example polyoxyethylene stearate, or condensation products of ethylene oxide with long-chain aliphatic alcohols, for example heptadecaethyleneoxycetanol, or condensation products of ethylene oxide with partial esters derived from fatty acids, for example polyoxyethylene sorbitan monooleate.

Oily suspensions may be formulated by suspending a therapeutic compound disclosed herein in admixture with (a) vegetable oils, such as, e.g., almond oil, arachis oil, avocado oil, canola oil, castor oil, coconut oil, corn oil, cottonseed oil, grape seed oil, hazelnut oil, hemp oil, linseed oil, olive oil, palm oil, peanut oil, rapeseed oil, rice bran oil, safflower oil, sesame oil, soybean oil, soya oil, sunflower oil, walnut oil, wheat germ oil, or a combination thereof, (b) a saturated fatty acid, an unsaturated fatty acid, or a combination thereof, such as, e.g., palmitic acid, stearic acid, oleic acid, linoleic acid, linolenic acid, or a combination thereof, (c) mineral oil such as, e.g., liquid paraffin, (d) surfactants or detergents. The oily suspensions may contain a thickening agent, for example beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the combined therapeutic compounds in admixture with a dispersing or wetting agent, suspending agent and one or more preservatives.

A therapeutic compound disclosed herein may be in the form of oil-in-water emulsions. The oily phase may be a vegetable oil as disclosed herein or a mineral oil as disclosed herein or mixtures thereof. Suitable emulsifying agents may be naturally occurring gums, such as, e.g., gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate.

A therapeutic compound disclosed herein, or a pharmaceutical composition disclosed herein, may also be incorporated into a drug delivery platform in order to achieve a controlled release profile over time. Such a drug delivery platform comprises a therapeutic compound disclosed herein dispersed within a polymer matrix, typically a biodegradable, bioerodible, and/or bioresorbable polymer matrix. As used herein, the term “polymer” refers to synthetic homo- or copolymers, naturally occurring homo- or copolymers, as well as synthetic modifications or derivatives thereof having a linear, branched or star structure. Copolymers can be arranged in any form, such as, e.g., random, block, segmented, tapered blocks, graft, or triblock. Polymers are generally condensation polymers. Polymers can be further modified to enhance their mechanical or degradation properties by introducing cross-linking agents or changing the hydrophobicity of the side residues. If crosslinked, polymers are usually less than 5% crosslinked, usually less than 1% crosslinked.

Suitable polymers include, without limitation, alginates, aliphatic polyesters, polyalkylene oxalates, polyamides, polyamidoesters, polyanhydrides, polycarbonates, polyesters, polyethylene glycol, polyhydroxyaliphatic carboxylic acids, polyorthoesters, polyoxaesters, polypeptides, polyphosphazenes, polysaccharides, and polyurethanes. The polymer usually comprises at least about 10% (w/w), at least about 20% (w/w), at least about 30% (w/w), at least about 40% (w/w), at least about 50% (w/w), at least about 60% (w/w), at least about 70% (w/w), at least about 80% (w/w), or at least about 90% (w/w) of the drug delivery platform. Examples of biodegradable, bioerodible, and/or bioresorbable polymers and methods useful to make a drug delivery platform are described in, e.g., Drost, et. al., Controlled Release Formulation, U.S. Pat. No. 4,756,911; Smith, et. al., Sustained Release Drug Delivery Devices, U.S. Pat. No. 5,378,475; Wong and Kochinke, Formulation for Controlled Release of Drugs by Combining Hyrophilic and Hydrophobic Agents, U.S. Pat. No. 7,048,946; Hughes, et. al., Compositions and Methods for Localized Therapy of the Eye, U.S. Patent Publication 2005/0181017; Hughes, Hypotensive Lipid-Containing Biodegradable Intraocular Implants and Related Methods, U.S. Patent Publication 2005/0244464; Altman, et al., Silk Fibroin Hydrogels and Uses Thereof, U.S. Patent Publication 2011/0008437; each of which is incorporated by reference in its entirety.

In aspects of this embodiment, a polymer composing the matrix is a polypeptide such as, e.g., silk fibroin, keratin, or collagen. In other aspects of this embodiment, a polymer composing the matrix is a polysaccharide such as, e.g., cellulose, agarose, elastin, chitosan, chitin, or a glycosaminoglycan like chondroitin sulfate, dermatan sulfate, keratan sulfate, or hyaluronic acid. In yet other aspects of this embodiment, a polymer composing the matrix is a polyester such as, e.g., D-lactic acid, L-lactic acid, racemic lactic acid, glycolic acid, caprolactone, and combinations thereof. One of ordinary skill in the art appreciates that the selection of a suitable polymer for forming a suitable disclosed drug delivery platform depends on several factors. The more relevant factors in the selection of the appropriate polymer(s), include, without limitation, compatibility of polymer with drug, desired release kinetics of drug, desired biodegradation kinetics of platform at implantation site, desired bioerodible kinetics of platform at implantation site, desired bioresorbable kinetics of platform at implantation site, in vivo mechanical performance of platform, processing temperatures, biocompatibility of platform, and patient tolerance. Other relevant factors that, to some extent, dictate the in vitro and in vivo behavior of the polymer include the chemical composition, spatial distribution of the constituents, the molecular weight of the polymer and the degree of crystallinity.

A drug delivery platform includes both a sustained release drug delivery platform and an extended release drug delivery platform. As used herein, the term “sustained release” refers to the release of a therapeutic compound disclosed herein over a period of about seven days or more. As used herein, the term “extended release” refers to the release of a therapeutic compound disclosed herein over a period of time of less than about seven days.

In aspects of this embodiment, a sustained release drug delivery platform releases a therapeutic compound disclosed herein with substantially zero order release kinetics over a period of, e.g., about 7 days after administration, about 15 days after administration, about 30 days after administration, about 45 days after administration, about 60 days after administration, about 75 days after administration, or about 90 days after administration. In other aspects of this embodiment, a sustained release drug delivery platform releases a therapeutic compound disclosed herein with substantially zero order release kinetics over a period of, e.g., at least 7 days after administration, at least 15 days after administration, at least 30 days after administration, at least 45 days after administration, at least 60 days after administration, at least 75 days after administration, or at least 90 days after administration.

In aspects of this embodiment, a sustained release drug delivery platform releases a therapeutic compound disclosed herein with substantially first order release kinetics over a period of, e.g., about 7 days after administration, about 15 days after administration, about 30 days after administration, about 45 days after administration, about 60 days after administration, about 75 days after administration, or about 90 days after administration. In other aspects of this embodiment, a sustained release drug delivery platform releases a therapeutic compound disclosed herein with substantially first order release kinetics over a period of, e.g., at least 7 days after administration, at least 15 days after administration, at least 30 days after administration, at least 45 days after administration, at least 60 days after administration, at least 75 days after administration, or at least 90 days after administration.

In aspects of this embodiment, a drug delivery platform releases a therapeutic compound disclosed herein with substantially zero order release kinetics over a period of, e.g., about 1 day after administration, about 2 days after administration, about 3 days after administration, about 4 days after administration, about 5 days after administration, or about 6 days after administration. In other aspects of this embodiment, a drug delivery platform releases a therapeutic compound disclosed herein with substantially zero order release kinetics over a period of, e.g., at most 1 day after administration, at most 2 days after administration, at most 3 days after administration, at most 4 days after administration, at most 5 days after administration, or at most 6 days after administration.

In aspects of this embodiment, a drug delivery platform releases a therapeutic compound disclosed herein with substantially first order release kinetics over a period of, e.g., about 1 day after administration, about 2 days after administration, about 3 days after administration, about 4 days after administration, about 5 days after administration, or about 6 days after administration. In other aspects of this embodiment, a drug delivery platform releases a therapeutic compound disclosed herein with substantially first order release kinetics over a period of, e.g., at most 1 day after administration, at most 2 days after administration, at most 3 days after administration, at most 4 days after administration, at most 5 days after administration, or at most 6 days after administration.

Aspects of the present specification disclose, in part, a method of treating an individual with a gender-biased immune disorder. In one embodiment, the method comprises the step of administering to an individual in need thereof a pharmaceutical composition disclosed herein, wherein administration reduces a symptom associated with the gender-biased immune disorder, thereby treating the individual.

Aspects of the present specification disclose, in part, use of a compound or a pharmaceutical composition disclosed herein to treat a gender-biased immune disorder.

A gender-biased immune disorder refers to an immune-based disease that preferentially affects one gender more than the other. In one embodiment, the incidence of a gender-biased immune disorder affects more women than men. In aspects of this embodiment, the incidence of a gender-biased immune disorder affects at least 2 times more women than men, at least 3 times more women than men, at least 4 times more women than men, at least 5 times more women than men, at least 6 times more women than men, at least 7 times more women than men, at least 2 times more women than men, at least 9 times more women than men, or at least 10 times more women than men. In another embodiment, the incidence of a gender-biased immune disorder affects more men than women. In aspects of this embodiment, the incidence of a gender-biased immune disorder affects at least 2 times more men than women, at least 3 times more men than women, at least 4 times more men than women, at least 5 times more men than women, at least 6 times more men than women, at least 7 times more men than women, at least 2 times more men than women, at least 9 times more men than women, or at least 10 times more men than women.

One example of a gender-biased immune disorders are autoimmune disorders. An autoimmune disorders arises from an overactive immune response of the body against substances and tissues normally present in the body resulting in a break in tolerance toward self-antigens. In other words, the body actually attacks its own cells because the immune system mistakes some part of the body as a pathogen and attacks it. Characterized by the development of pathogenic T cell populations infiltrating the target organ or tissue, autoimmune disorders may be restricted to certain organs or involve a particular tissue in different places. An autoimmune disorder can be a systemic autoimmune disorder or an organ-specific autoimmune disorder.

Non-limiting examples of an autoimmune disorder that can be treated using a compound or a pharmaceutical composition disclosed herein include an acute disseminated encephalomyelitis (ADEM), an Addison's disease, an allergy, allergic rhinitis, an Alzheimer's disease, alopecia areata, amyotrophic lateral sclerosis, anemia, ankylosing spondylitis, an anti-GBM nephritis, an anti-TBM nephritis, an anti-phospholipid antibody syndrome (APS), aplastic anemia, an arthritis such as, e.g., a monoarthritis, an oligoarthritis, or a polyarthritis like an osteoarthritis, a rheumatoid arthritis, a juvenile idiopathic arthritis, a septic arthritis, a spondyloarthropathy, a gout, a pseudogout, or Still's disease, an asthma, atopic allergy, an autoimmune deficiency syndrome (AIDS), an autoimmune hemolytic anemia, an autoimmune hepatitis, an autoimmune inner ear disease, an autoimmune lymphoproliferative syndrome (ALPS), a Balo disease, a Barrett's esophagus, a Behcet's disease, a Berger's disease (IgA nephropathy), a bullous pemphigoid, a bursitis, a cardiomyopathy, a celiac disease, a Chagas disease, a chronic obstructive pulmonary disease (COPD), Churg Strauss syndrome, cicatricial pemphigoid, Cogan's syndrome, cold agglutunin disease, a cranial arteritis, a CREST syndrome, a Cushing's syndrome, a Dego's disease, a dermatitis, a dermatomyositis, a devic disease, a diabetes mellitus type 1 (IDDM), a diabetes mellitus type 2, Dressler's syndrome, eczema, eosinophilic fasciitis, epidermolysis bullosa acquisita, an endometriosis, essential mixed cryoglobulinemia, Evan's syndrome, fibromyalgia, fibrosing alveolitis, a gastrointestinal disorder such as, e.g., digestive dysfunction, diverticulitis, diverticulosis, an irritable bowel disease or an inflammatory bowel disease like Crohn's disease or an ulcerative colitis, a giant cell arteritis, a glomerulonephritis, a Goodpasture's syndrome, a Graves' disease, a Guillain-Barré syndrome (GBS), a Hashimoto's thyroiditis, a hemolytic anemia, a hemorrhoids, a Henoch-Schönlein purpura, a hepatitis, a hiatal hernia, a hidradenitis suppurativa, a Hughes syndrome, an idiopathic adrenal atrophy, an idiopathic pulmonary fibrosis, an idiopathic thrombocytopenia purpura, inflammatory demylinating polyneuropathy, an interstitial cystitis, a Kawasaki's disease, a leaky gut syndrome, a lichen planus, a lupoid hepatitis, a lupus, such as, e.g., a discoid lupus erythematosus, a drug-induced lupus erythematosus, a lupus nephritis, a neonatal lupus, a subacute cutaneous lupus erythematosus, or a systemic lupus erythematosus, a Lyme disease, a Meniere's disease, a mixed connective tissue disease, a morphea, a multiple myeloma, a multiple sclerosis (MS), a myasthenia gravis, a myopathy such as, e.g., a dermatomyositis, an inclusion body myositis, or a polymyositis, a myositis, a narcolepsy, a neuromyotonia, an ocular cicatricial pemphigoid, an osteoporosis, a Parkinson's disease, a pars planitis, a pemphigus vulgaris, a pernicious anaemia, a polyglandular autoimmune syndrome, a polymyalgia rheumatic, a polymyositis, a primary biliary cirrhosis, a primary sclerosing cholangitis, a proctitis, a Raynaud's phenomenon, a Reiter's syndrome, a recurrent disseminated encephalomyelitis, a rheumatic fever, a schizophrenia, a scleritis, a scleroderma, a Sjögren's syndrome, a skin disorder such as, e.g., dermatitis, an eczema, a statis dermatitis, a hidradenitis suppurativa, a psoriasis, a rosacea or a sarcoidosis, a sticky blood syndrome, a stiff man syndrome, a Still's disease, a sydenham chorea, a tenosynovitis, a uveitis, vasculitis such as, e.g., a Buerger's disease, a cerebral vasculitis, a Churg-Strauss arteritis, a cryoglobulinemia, an essential cryoglobulinemic vasculitis, a giant cell arteritis, a Golfers vasculitis, a Henoch-Schönlein purpura, a hypersensitivity vasculitis, a Kawasaki disease, a microscopic polyarteritis/polyangiitis, a polyarteritis nodosa, a polymyalgia rheumatica (PMR), a rheumatoid vasculitis, a Takayasu arteritis, a temporal arteritis, or a Wegener's granulomatosis, a vitiligo, a Wilson's syndrome, or a transplant rejection include a hyperacute rejection, an acute rejection, or a chronic rejection, as well as, a graft-versus-host-disease.

Gender-biased immune disorders that primarily affects women include, without limitation, Hashimoto's thyroiditis, systemic lupus erythematosus (SLE), Sjogren syndrome, Graves' disease, autoimmune hepatitis, rheumatoid arthritis, osteoarthritis, osteoporosis, chronic fatigue syndrome, fibromyalgia, lupus, irritable bowel syndrome, cataracts, asthma, interstitial cystitis, Thrombocytopenia purpura, myasthenia gravis, multiple sclerosis, systemic sclerosis, and dermatomyositis. Gender-biased immune disorders that primarily affects men include, without limitation primary sclerosing cholangitis, ankylosing spondylitis, myocarditis, dilated cardiomyopathy, pernicious anemia, type 1 diabetes mellitus, gastritis, Wegener's granulomatosis, idiopathic pulmonary fibrosis, Crohn's disease, and psoriasis.

An individual preferentially mounts either a Th1-driven immune response or a Th2-driven immune response. Based primarily on genetic make-up, an individual is more susceptible to a Th1 or Th2 driven disease. In one embodiment, a gender-biased immune disorder is exclusively a Th1-based disorder. In one embodiment, a gender-biased immune disorder is exclusively a Th2-based disorder. In another embodiment, a gender-biased immune disorder is not a Th1-based disorder. In another embodiment, a gender-biased immune disorder is not a Th2-based disorder.

In an embodiment, a gender-biased immune disorder is a Th1-based immune disorder. A Th1-based immune disorder may be one associated with abnormal or pathologically high levels of CD8⁺ helper T cell activity. Such activity stimulated a cellular immune response, results in increased levels of Th1-type pro-inflammatory cytokines, and attacks and destroys antigen presenting cells. Examples of a Th1-based immune disorder include, without limitation, rheumatoid arthritis, reactive arthritis, multiple sclerosis, Chagus disease, Crohn's disease, psoriasis, psoriatic arthritis, juvenile-onset rheumatoid arthritis, uvetis, age-related macular degeneration, pandemic flu, respiratory syncytical virus, cystic fibrosis, genital herpes, sepsis, septic shock, dengue hemorrhagic fever, type 1 diabetes, endometrosis, and prostatis.

In an embodiment, a gender-biased immune disorder is a Th2-based immune disorder. A Th2-based immune disorder may be one associate with abnormal or pathologic B cell activity resulting in complement-driven inflammation. Examples of a Th2-based immune disorder include, without limitation, asthma, allergic asthma, systemic lupus erythematosus, cutaneous lupus, atopic disease, eczema, allergic rhinitis, ulcerative colitis, scleroderma, and sarcoidosis.

A gender-biased immune disorder, like as Th1-based or Th2-based immune disorder, can be classified based on its hypersensitivity. Hypersensitivity (also called hypersensitivity reaction or intolerance) refers to undesirable reactions produced by the normal immune system, including allergies and autoimmunity. These reactions may be damaging, uncomfortable, or occasionally fatal. Hypersensitivity reactions require a pre-sensitized (immune) state of the host. They are classified in five groups.

Type I hypersensitivity (or immediate hypersensitivity) is an allergic reaction provoked by reexposure to a specific type of antigen referred to as an allergen. In type 1 hypersensitivity, an antigen is presented to CD4⁺ Th2 cells specific to the antigen that stimulate B-cell production of IgE antibodies also specific to the antigen. The difference between a normal infectious immune response and a type 1 hypersensitivity response is that in type 1 hypersensitivity the antibody is IgE instead of IgA, IgG, or IgM. During sensitization, the IgE antibodies bind to Fcε receptors on the surface of tissue mast cells and blood basophils. Mast cells and basophils coated by IgE antibodies are “sensitized.” Later exposure to the same allergen cross-links the bound IgE on sensitized cells, resulting in degranulation and the secretion of pharmacologically active mediators such as histamine, leukotriene (LTC4 and LTD4), and prostaglandin that act on the surrounding tissues. The principal effects of these products are vasodilation and smooth-muscle contraction. Type 1 hypersensitivity can be further classified into an immediate and late-phase reaction. The immediate hypersensitivity reaction occurs minutes after exposure and includes release of vasoactive amines and lipid mediators, whereas the late-phase reaction occurs 2-4 hours after exposure and includes the release of cytokines. Examples of Type I hypersensitivity include allergic asthma, allergic conjunctivitis, allergic rhinitis (“hay fever”), allergic urticarial, anaphylaxis, angioedema, asthma, atopy, atopic eczema, cephalosporin allergy, eosinophilia, food allergy (such as, e.g., milk, egg, peanut, tree nut, seafood, soy, wheat), penicillin allergy, and urticaria (hives).

Type II hypersensitivity (or cytotoxic hypersensitivity) is an antibody-dependent reaction. In type II hypersensitivity, antibodies produced by the immune response bind to antigens on the patient's own cell surfaces. The antigens recognized in this way may either be intrinsic (“self” antigen, innately part of the patient's cells) or extrinsic (adsorbed onto the cells during exposure to some foreign antigen, possibly as part of infection with a pathogen). These cells are recognized by macrophages or dendritic cells, which act as antigen-presenting cells. This causes a B cell response, wherein antibodies are produced against the foreign antigen. Examples of Type II hypersensitivity include autoimmune hemolytic anemia, bullous pemphigoid, erythroblastosis fetalis, Goodpasture's syndrome, thrombocytopenia, membranous neuropathy, pemphigus vulgaris, and rheumatic fever.

Type III hypersensitivity occurs when antigen-antibody complexes that are not adequately cleared by innate immune cells accumulate, giving rise to an inflammatory response and attraction of leukocytes. In type III hypersensitivity, there is an excess of antigen which leads to small immune complexes being formed that do not fix complement and are not cleared from the circulation. It is characterized by solvent antigens that are not bound to cell surfaces (which is the case in type II hypersensitivity). When these antigens bind antibodies, immune complexes of different sizes form. Large complexes can be cleared by macrophages but macrophages have difficulty in the disposal of small immune complexes. These immune complexes insert themselves into small blood vessels, joints, and glomeruli, causing symptoms. Unlike the free variant, a small immune complex bound to sites of deposition (like blood vessel walls) are far more capable of interacting with complement; these medium-sized complexes, formed in the slight excess of antigen, are viewed as being highly pathogenic. Such depositions in tissues often induce an inflammatory response, and can cause damage wherever they precipitate. The cause of damage is as a result of the action of cleaved complement anaphylotoxins C3a and C5a, which, respectively, mediate the induction of granule release from mast cells (from which histamine can cause urticaria), and recruitment of inflammatory cells into the tissue (mainly those with lysosomal action, leading to tissue damage through frustrated phagocytosis by PMNs and macrophages). Examples of Type III hypersensitivity include those elicited by a foreign body like arthus reaction, extrinsic allergic alveolitis (hypersensitivity pneumonitis), farmers lung, Henoch-Schónlein purpura, hypersensitivity vasculitis, post-streptococcal glomerulonephritis, reactive arthritis, and serum sickness; and those elicited as an autoimmune response like lupus nephritis, rheumatoid arthritis, subacute bacterial endocarditis, and systemic lupus erythematosus.

Type IV hypersensitivity (delayed-type hypersensitivity) is a type of cell-mediated response (not antibody mediated). In type IV hypersensitivity, CD4+ helper T cells recognize antigen in a complex with Class 2 major histocompatibility complex. The antigen-presenting cells in this case are macrophages that secrete IL-12, which stimulates the proliferation of further CD4+ Th1 cells. CD4+ T cells secrete IL-2 and IFγ, further inducing the release of other Th1 cytokines, thus mediating the immune response. Activated CD8+ T cells destroy target cells on contact, whereas activated macrophages produce hydrolytic enzymes and, on presentation with certain intracellular pathogens, transform into multinucleated giant cells. Type IV hypersensitivity is often called delayed type hypersensitivity as the reaction takes two to three days to develop. Examples of Type III hypersensitivity include those elicited by a foreign body like allergic contact dermatitis and tuberculosis; and those elicited as an autoimmune response like Coeliac disease, Crohn's disease, diabetes mellitus type 1, Hashimoto's thyroiditis, Giant-cell arteritis, Guillain-Barré syndrome, multiple sclerosis, rheumatoid arthritis, and graft versus host disease (chronic transplant rejection and transfusion-associated graft versus host disease).

Type V hypersensitivity (receptor-mediated hypersensitivity) is an antibody-dependent reaction. In type V hypersensitivity, instead of binding to cell surface components (like Type II), the antibodies recognize and bind to the cell surface receptors, which either prevents the intended ligand binding with the receptor or mimics the effects of the ligand, thus impairing cell signaling. Examples of Type V hypersensitivity include Graves' disease and myasthenia gravis.

Aspects of the present specification disclose, in part, treating an individual suffering from a gender-biased immune disorder. As used herein, the term “treating,” refers to reducing or eliminating in an individual a clinical symptom of a gender-biased immune disorder; or delaying or preventing in an individual the onset of a clinical symptom of a gender-biased immune disorder. For example, the term “treating” can mean reducing a symptom of a gender-biased immune disorder by, e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 100%. As another example, the term “treating” can mean controlling a symptom of a gender-biased immune disorder such as, e.g., reducing the number of symptoms per given time period and/or the severity of a symptom. The actual symptoms associated with a gender-biased immune disorder are well known and can be determined by a person of ordinary skill in the art by taking into account factors, including, without limitation, the location of the gender-biased immune disorder, the cause of the gender-biased immune disorder, the severity of the gender-biased immune disorder, and/or the cells, tissue or organ affected by the gender-biased immune disorder. Those of skill in the art will know the appropriate symptoms or indicators associated with a specific type of a gender-biased immune disorder and will know how to determine if an individual is a candidate for treatment as disclosed herein.

Symptoms of a gender-biased immune disorder include, without limitation, allergy, inflammation, immune problem, drowsiness, fainting, nausea, fatigue, fever and high body temperature, extreme sensitivity to cold, malaise, burning pain, abdominal cramping, abdominal swelling, abdominal tenderness, abdominal pain, muscle tone loss, muscle cramping, muscle weakness, muscle stiffness, muscle swelling, muscle tenderness, muscle pain, joint weakness, joint stiffness, joint swelling, joint tenderness, joint inflammation, joint pain, arthritis, temporary loss of cognitive abilities, cognitive fogging, dizziness, drowsiness, chronic anxiety, chronic bloating, chronic constipation, chronic depression, chronic diarrhea, chronic fatigue syndrome (CFS), chronic headache, chronic indigestion, chronic insomnia, chronic irritability, chronic migraine, chronic nausea, chronic pain, malar blush, disoid rash, alopecia, Raynaud phenomenon, livedo reticularis, panniculitis (lupus profundus), bullous lesions, vasculitic purpura, telangiectasias, urticaria, photosensitivity, a skin lesion, skin disorder, nasal inflammation, hemorrhoids, rectal bleeding, blood in stool, constipation, intestinal bleeding, intestinal obstruction, acid reflux, diarrhea, digestive problems, indigestion, fistula, mesenteric vasculitis, bowel infarction, flatulence, excess gas, hematuria, proteinuria, uremia, nephritis, glomerulonephritis, edema, bloating, weight gain, hyperlipidemia, high cholesterol, pericarditis, hypertension, vasculitis, myocarditis, endocarditis, atherosclerosis, dyspnea, angina, encephalopathy, cerebritis, headache, migraine, cognitive dysfunction, mood disorder, confusion, insomnia, irritability, depression, memory loss, cerebrovascular disease, seizures, polyneuropathy, anxiety disorder, agitation, psychosis, personality disorder, intracranial pressure, papilledema, nerve paresis, paranoia, hallucination, Guillain-Barré syndrome, aseptic meningitis, autonomic disorder, demyelinating syndrome, mononeuropathy, movement disorder, myasthenia gravis, myelopathy, cranial neuropathy, plexopathy, aseptic meningitis, myelopathy, optic neuropathy, a demyelinating disorder, myelitis, paraparesis, ischemia, transient ischemic attack, stroke, pleuritis, pleurisy, pleural effusion, pneumonitis, hemoptysis, chronic diffuse interstitial lung disease, pulmonary hypertension, low or high blood pressure, pulmonary emboli, pulmonary hemorrhage, shrinking lung syndrome, anemia, thrombocytopenia, leucopenia, lymphopenia, phlebitis, menstrual problem, miscarriage, tissue degeneration, ulcer, dry eye, vision loss, peritonitis, gland or lymph problem, hormonal imbalance, pancreatitis, hormonal imbalance, infertility or reduced sex drive (low libido), low blood sugar level, high blood sugar level, blood sugar changes, jaundice, abnormal growths, abnormal tissue formation, polyps, abscess, hair loss, general infection, bacterial infection, fungal infection, parasitic infection, and/or yeast infection, and depending on the type of autoimmune disease, an increase in the size of an organ or tissue, or the destruction of an organ or tissue. Non-limiting examples of an inflammation symptom reduced by a method of treating an autoimmune disorder disclosed herein include edema, hyperemia, erythema, bruising, tenderness, stiffness, swollenness, fever, a chill, congestion of the respiratory tract including nose, and bronchi, congestion of a sinus, a breathing problem, fluid retention, a blood clot, a loss of appetite, an increased heart rate, a formation of granulomas, fibrinous, pus, or non-viscous serous fluid, a formation of an ulcer, or pain.

In one embodiment, a therapeutic compound disclosed herein reduces a symptom associated with a gender-biased immune disorder. In aspects of this embodiment, a therapeutic compound disclosed herein reduces a symptom associated with a gender-biased immune disorder by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In other aspects of this embodiment, a therapeutic compound disclosed herein reduces a symptom associated with a gender-biased immune disorder by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In another embodiment, a therapeutic compound disclosed herein reduces the frequency of a symptom of a gender-biased immune disorder incurred over a given time period. In aspects of this embodiment, a therapeutic compound disclosed herein reduces the frequency of a symptom of a gender-biased immune disorder incurred over a given time period by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In other aspects of this embodiment, a therapeutic compound disclosed herein reduces the frequency of a symptom of a gender-biased immune disorder incurred over a given time period by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In another embodiment, a therapeutic compound disclosed herein reduces the number of symptoms of a gender-biased immune disorder incurred over a given time period. In aspects of this embodiment, a therapeutic compound disclosed herein reduces the number of symptoms of a gender-biased immune disorder incurred over a given time period by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In other aspects of this embodiment, a therapeutic compound disclosed herein reduces the number of symptoms of a gender-biased immune disorder incurred over a given time period by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In another embodiment, a therapeutic compound disclosed herein reduces the severity of a symptom of a gender-biased immune disorder. In aspects of this embodiment, a therapeutic compound disclosed herein reduces the severity of a symptom of a gender-biased immune disorder by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In other aspects of this embodiment, a therapeutic compound disclosed herein reduces the severity of a symptom of a gender-biased immune disorder by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

A composition or compound is administered to an individual. An individual is typically a human being. An individual may be a female, a male or either. In aspects of this embodiment, a method or use disclosed herein is for the treatment of only females suffering from a gender-biased immune disorder. In other aspects of this embodiment, a method or use disclosed herein is for the treatment of only males suffering from a gender-biased immune disorder. Typically, any individual who is a candidate for a conventional treatment is a candidate for a gender-biased immune disorder treatment disclosed herein. Pre-operative evaluation typically includes routine history and physical examination in addition to thorough informed consent disclosing all relevant risks and benefits of the procedure.

A pharmaceutical composition disclosed herein may comprise a therapeutic compound in a therapeutically effective amount. As used herein, the term “effective amount” is synonymous with “therapeutically effective amount”, “effective dose”, or “therapeutically effective dose” and when used in reference to treating a gender-biased immune disorder refers to the minimum dose of a therapeutic compound disclosed herein necessary to achieve the desired therapeutic effect and includes a dose sufficient to reduce a symptom associated with a gender-biased immune disorder. The effectiveness of a therapeutic compound disclosed herein in treating a gender-biased immune disorder can be determined by observing an improvement in an individual based upon one or more clinical symptoms, and/or physiological indicators associated with the gender-biased immune disorder. An improvement in a gender-biased immune disorder also can be indicated by a reduced need for a concurrent therapy. In addition, a therapeutically effective amount is one where an individual does not begin to manifest secondary sex characteristics.

The appropriate effective amount of a therapeutic compound disclosed herein to be administered to an individual for a particular immune disorder can be determined by a person of ordinary skill in the art by taking into account factors, including, without limitation, the type of immune disorder, the location of the gender-biased immune disorder, the cause of the gender-biased immune disorder, the severity of the gender-biased immune disorder, the degree of relief desired, the duration of relief desired, the particular therapeutic compound used, the rate of excretion of the therapeutic compound used, the pharmacodynamics of the therapeutic compound used, the nature of the other compounds to be included in the composition, the particular route of administration, the particular characteristics, history and risk factors of the patient, such as, e.g., age, weight, general health and the like, or any combination thereof. Additionally, where repeated administration of a therapeutic compound is used, an effective amount of a therapeutic compound will further depend upon factors, including, without limitation, the frequency of administration, the half-life of the therapeutic compound, or any combination thereof. In is known by a person of ordinary skill in the art that an effective amount of a therapeutic compound disclosed herein can be extrapolated from in vitro assays and in vivo administration studies using animal models prior to administration to humans.

Wide variations in the necessary effective amount are to be expected in view of the differing efficiencies of the various routes of administration. For instance, oral administration of a therapeutic compound disclosed herein generally would be expected to require higher dosage levels than administration by inhalation. Similarly, systemic administration of a therapeutic compound disclosed herein would be expected to require higher dosage levels than a local administration. Variations in these dosage levels can be adjusted using standard empirical routines of optimization, which are well-known to a person of ordinary skill in the art. The precise therapeutically effective dosage levels and patterns are preferably determined by the attending physician in consideration of the above-identified factors. One skilled in the art will recognize that the condition of the individual can be monitored throughout the course of therapy and that the effective amount of a therapeutic compound disclosed herein that is administered can be adjusted accordingly.

In aspects of this embodiment, a therapeutically effective amount of a therapeutic compound disclosed herein reduces a symptom associated with a gender-biased immune disorder by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a therapeutic compound disclosed herein reduces a symptom associated with a gender-biased immune disorder by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a therapeutic compound disclosed herein reduces a symptom associated with a gender-biased immune disorder by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In other aspects of this embodiment, a therapeutically effective amount of a therapeutic compound disclosed herein generally is in the range of about 0.01 ng/kg/day to about 100 ng/kg/day. In aspects of this embodiment, an effective amount of a therapeutic compound disclosed herein may be, e.g., at least 0.01 ng/kg/day, at least 0.1 ng/kg/day, at least 1.0 ng/kg/day, at least 5.0 ng/kg/day, at least 10 ng/kg/day, at least 15 ng/kg/day, at least 20 ng/kg/day, at least 25 ng/kg/day, at least 30 ng/kg/day, at least 35 ng/kg/day, at least 40 ng/kg/day, at least 45 ng/kg/day, or at least 50 ng/kg/day. In other aspects of this embodiment, an effective amount of a therapeutic compound disclosed herein may be in the range of, e.g., about 0.01 ng/kg/day to about 10 ng/kg/day, about 0.01 ng/kg/day to about 15 ng/kg/day, about 0.01 ng/kg/day to about 20 ng/kg/day, about 0.01 ng/kg/day to about 25 ng/kg/day, about 0.01 ng/kg/day to about 30 ng/kg/day, about 0.01 ng/kg/day to about 35 ng/kg/day, about 0.01 ng/kg/day to about 40 ng/kg/day, about 0.01 ng/kg/day to about 45 ng/kg/day, about 0.01 ng/kg/day to about 50 ng/kg/day, about 0.01 ng/kg/day to about 75 ng/kg/day, or about 0.01 ng/kg/day to about 100 ng/kg/day. In yet other aspects of this embodiment, an effective amount of a therapeutic compound disclosed herein may be in the range of, e.g., about 0.1 ng/kg/day to about 10 ng/kg/day, about 0.1 ng/kg/day to about 15 ng/kg/day, about 0.1 ng/kg/day to about 20 ng/kg/day, about 0.1 ng/kg/day to about 25 ng/kg/day, about 0.1 ng/kg/day to about 30 ng/kg/day, about 0.1 ng/kg/day to about 35 ng/kg/day, about 0.1 ng/kg/day to about 40 ng/kg/day, about 0.1 ng/kg/day to about 45 ng/kg/day, about 0.1 ng/kg/day to about 50 ng/kg/day, about 0.1 ng/kg/day to about 75 ng/kg/day, or about 0.1 ng/kg/day to about 100 ng/kg/day.

In still other aspects of this embodiment, an effective amount of a therapeutic compound disclosed herein may be in the range of, e.g., about 1 ng/kg/day to about 10 ng/kg/day, about 1 ng/kg/day to about 15 ng/kg/day, about 1 ng/kg/day to about 20 ng/kg/day, about 1 ng/kg/day to about 25 ng/kg/day, about 1 ng/kg/day to about 30 ng/kg/day, about 1 ng/kg/day to about 35 ng/kg/day, about 1 ng/kg/day to about 40 ng/kg/day, about 1 ng/kg/day to about 45 ng/kg/day, about 1 ng/kg/day to about 50 ng/kg/day, about 1 ng/kg/day to about 75 ng/kg/day, or about 1 ng/kg/day to about 100 ng/kg/day. In other aspects of this embodiment, an effective amount of a therapeutic compound disclosed herein may be in the range of, e.g., about 5 ng/kg/day to about 10 ng/kg/day, about 5 ng/kg/day to about 15 ng/kg/day, about 5 ng/kg/day to about 20 ng/kg/day, about 5 ng/kg/day to about 25 ng/kg/day, about 5 ng/kg/day to about 30 ng/kg/day, about 5 ng/kg/day to about 35 ng/kg/day, about 5 ng/kg/day to about 40 ng/kg/day, about 5 ng/kg/day to about 45 ng/kg/day, about 5 ng/kg/day to about 50 ng/kg/day, about 5 ng/kg/day to about 75 ng/kg/day, or about 5 ng/kg/day to about 100 ng/kg/day.

In aspects of this embodiment, an effective amount of a therapeutic compound disclosed herein may be in the range of about 1 ng/day to about 10,000 ng/day. In other aspects of this embodiment an effective amount of a therapeutic compound disclosed herein may be, e.g., at least 1 ng/day, at least 10 ng/day, at least 25 ng/day, at least 50 ng/day, at least 100 ng/day, at least 150 ng/day, at least 200 ng/day, at least 250 ng/day, at least 300 ng/day, at least 350 ng/day, at least 400 ng/day, at least 450 ng/day, at least 500 ng/day, at least 550 ng/day, at least 600 ng/day, at least 650 ng/day, at least 700 ng/day, at least 750 ng/day, at least 800 ng/day, at least 850 ng/day, at least 900 ng/day, at least 950 ng/day, at least 1,000 ng/day, at least 1,50 ng/day, at least 1,100 ng/day, at least 1,150 ng/day, at least 1,200 ng/day, at least 1,250 ng/day, at least 1,300 ng/day, at least 1,350 ng/day, at least 1,400 ng/day, at least 1,450 ng/day, at least 1,500 ng/day, at least 1,600 ng/day, at least 1,700 ng/day, at least 1,800 ng/day, at least 1,900 ng/day, at least 2,000 ng/day, at least 2,100 ng/day, at least 2,200 ng/day, at least 2,300 ng/day, at least 2,400 ng/day, at least 2,500 ng/day, at least 2,600 ng/day, at least 2,700 ng/day, at least 2,800 ng/day, at least 2,900 ng/day, at least 3,000 ng/day, at least 3,500 ng/day, at least 4,000 ng/day, at least 4,500 ng/day, at least 5,000 ng/day, at least 5,500 ng/day, at least 6,000 ng/day, at least 6,500 ng/day, at least 7,000 ng/day, at least 7,500 ng/day, at least 8,000 ng/day, at least 8,500 ng/day, at least 9,000 ng/day, at least 9,500 ng/day, or at least 10,000 ng/day.

In other aspects of this embodiment an effective amount of a therapeutic compound disclosed herein may be, e.g., at most 1 ng/day, at most 10 ng/day, at most 25 ng/day, at most 50 ng/day, at most 100 ng/day, at most 150 ng/day, at most 200 ng/day, at most 250 ng/day, at most 300 ng/day, at most 350 ng/day, at most 400 ng/day, at most 450 ng/day, at most 500 ng/day, at most 550 ng/day, at most 600 ng/day, at most 650 ng/day, at most 700 ng/day, at most 750 ng/day, at most 800 ng/day, at most 850 ng/day, at most 900 ng/day, at most 950 ng/day, at most 1,000 ng/day, at most 1,50 ng/day, at most 1,100 ng/day, at most 1,150 ng/day, at most 1,200 ng/day, at most 1,250 ng/day, at most 1,300 ng/day, at most 1,350 ng/day, at most 1,400 ng/day, at most 1,450 ng/day, at most 1,500 ng/day, at most 1,600 ng/day, at most 1,700 ng/day, at most 1,800 ng/day, at most 1,900 ng/day, at most 2,000 ng/day, at most 2,100 ng/day, at most 2,200 ng/day, at most 2,300 ng/day, at most 2,400 ng/day, at most 2,500 ng/day, at most 2,600 ng/day, at most 2,700 ng/day, at most 2,800 ng/day, at most 2,900 ng/day, at most 3,000 ng/day, at most 3,500 ng/day, at most 4,000 ng/day, at most 4,500 ng/day, at most 5,000 ng/day, at most 5,500 ng/day, at most 6,000 ng/day, at most 6,500 ng/day, at most 7,000 ng/day, at most 7,500 ng/day, at most 8,000 ng/day, at most 8,500 ng/day, at most 9,000 ng/day, at most 9,500 ng/day, or at most 10,000 ng/day.

In yet other aspects of this embodiment an effective amount of a therapeutic compound disclosed herein may be between, e.g., about 1 ng/day to about 1,000 ng/day, about 10 ng/day to about 1,000 ng/day, about 25 ng/day to about 1,000 ng/day, about 50 ng/day to about 1,000 ng/day, about 100 ng/day to about 1,000 ng/day, about 150 ng/day to about 1,000 ng/day, about 200 ng/day to about 1,000 ng/day, about 250 ng/day to about 1,000 ng/day, about 300 ng/day to about 1,000 ng/day, about 350 ng/day to about 1,000 ng/day, about 400 ng/day to about 1,000 ng/day, about 450 ng/day to about 1,000 ng/day, about 500 ng/day to about 1,000 ng/day, about 50 ng/day to about 1,500 ng/day, about 100 ng/day to about 1,500 ng/day, about 150 ng/day to about 1,500 ng/day, about 200 ng/day to about 1,500 ng/day, about 250 ng/day to about 1,500 ng/day, about 300 ng/day to about 1,500 ng/day, about 350 ng/day to about 1,500 ng/day, about 400 ng/day to about 1,500 ng/day, about 450 ng/day to about 1,500 ng/day, about 500 ng/day to about 1,500 ng/day, about 1,000 ng/day to about 3,000 ng/day, about 1,100 ng/day to about 3,000 ng/day, about 1,200 ng/day to about 3,000 ng/day, about 1,3000 ng/day to about 3,000 ng/day, about 1,400 ng/day to about 3,000 ng/day, about 1,500 ng/day to about 3,000 ng/day, about 1,600 ng/day to about 3,000 ng/day, about 1,700 ng/day to about 3,000 ng/day, about 1,800 ng/day to about 3,000 ng/day, about 1,900 ng/day to about 3,000 ng/day, about 2,000 ng/day to about 3,000 ng/day, about 1,000 ng/day to about 4,000 ng/day, about 1,100 ng/day to about 4,000 ng/day, about 1,200 ng/day to about 4,000 ng/day, about 1,3000 ng/day to about 4,000 ng/day, about 1,400 ng/day to about 4,000 ng/day, about 1,500 ng/day to about 4,000 ng/day, about 1,600 ng/day to about 4,000 ng/day, about 1,700 ng/day to about 4,000 ng/day, about 1,800 ng/day to about 4,000 ng/day, about 1,900 ng/day to about 4,000 ng/day, about 2,000 ng/day to about 4,000 ng/day, about 2,500 ng/day to about 4,000 ng/day, about 3,000 ng/day to about 4,000 ng/day, about 1,000 ng/day to about 5,000 ng/day, about 1,100 ng/day to about 5,000 ng/day, about 1,200 ng/day to about 5,000 ng/day, about 1,3000 ng/day to about 5,000 ng/day, about 1,400 ng/day to about 5,000 ng/day, about 1,500 ng/day to about 5,000 ng/day, about 1,600 ng/day to about 5,000 ng/day, about 1,700 ng/day to about 5,000 ng/day, about 1,800 ng/day to about 5,000 ng/day, about 1,900 ng/day to about 5,000 ng/day, about 2,000 ng/day to about 5,000 ng/day, about 2,500 ng/day to about 5,000 ng/day, about 3,000 ng/day to about 5,000 ng/day, about 3,500 ng/day to about 5,000 ng/day, about 4,000 ng/day to about 5,000 ng/day, about 1,000 ng/day to about 6,000 ng/day, about 1,100 ng/day to about 6,000 ng/day, about 1,200 ng/day to about 6,000 ng/day, about 1,3000 ng/day to about 6,000 ng/day, about 1,400 ng/day to about 6,000 ng/day, about 1,500 ng/day to about 6,000 ng/day, about 1,600 ng/day to about 6,000 ng/day, about 1,700 ng/day to about 6,000 ng/day, about 1,800 ng/day to about 6,000 ng/day, about 1,900 ng/day to about 6,000 ng/day, about 2,000 ng/day to about 6,000 ng/day, about 2,500 ng/day to about 6,000 ng/day, about 3,000 ng/day to about 6,000 ng/day, about 3,500 ng/day to about 6,000 ng/day, about 4,000 ng/day to about 6,000 ng/day, about 4,500 ng/day to about 6,000 ng/day, or about 5,000 ng/day to about 6,000 ng/day, about 2,000 ng/day to about 7,000 ng/day, about 2,500 ng/day to about 7,000 ng/day, about 3,000 ng/day to about 7,000 ng/day, about 3,500 ng/day to about 7,000 ng/day, about 4,000 ng/day to about 7,000 ng/day, about 4,500 ng/day to about 7,000 ng/day, about 5,000 ng/day to about 7,000 ng/day, about 5,500 ng/day to about 7,000 ng/day, or about 6,000 ng/day to about 7,000 ng/day, about 2,000 ng/day to about 8,000 ng/day, about 2,500 ng/day to about 8,000 ng/day, about 3,000 ng/day to about 8,000 ng/day, about 3,500 ng/day to about 8,000 ng/day, about 4,000 ng/day to about 8,000 ng/day, about 4,500 ng/day to about 8,000 ng/day, about 5,000 ng/day to about 8,000 ng/day, about 5,500 ng/day to about 8,000 ng/day, about 6,000 ng/day to about 8,000 ng/day, about 6,500 ng/day to about 8,000 ng/day, or about 7,000 ng/day to about 8,000 ng/day.

In aspects of this embodiment, a therapeutically effective amount of a therapeutic compound disclosed herein generally is in the range of about 0.001 mg/kg/day to about 10 mg/kg/day. In other aspects of this embodiment, a therapeutically effective amount of a therapeutic compound disclosed herein may be, e.g., at least 0.001 mg/kg/day, at least 0.0025 mg/kg/day, at least 0.005 mg/kg/day, at least 0.0075 mg/kg/day, at least 0.01 mg/kg/day, at least 0.025 mg/kg/day, at least 0.05 mg/kg/day, at least 0.075 mg/kg/day, at least 0.1 mg/kg/day, at least 0.25 mg/kg/day, at least 0.5 mg/kg/day, at least 0.75 mg/kg/day, at least 1.0 mg/kg/day, at least 2.5 mg/kg/day, at least 5.0 mg/kg/day, at least 7.5 mg/kg/day, or at least 10 mg/kg/day. In yet other aspects of this embodiment, a therapeutically effective amount of a therapeutic compound disclosed herein may be, e.g., about 0.001 mg/kg/day to about 0.1 mg/kg/day, about 0.001 mg/kg/day to about 0.5 mg/kg/day, about 0.001 mg/kg/day to about 1 mg/kg/day, about 0.001 mg/kg/day to about 5 mg/kg/day, about 0.001 mg/kg/day to about 10 mg/kg/day, about 0.01 mg/kg/day to about 0.1 mg/kg/day, about 0.01 mg/kg/day to about 0.5 mg/kg/day, about 0.01 mg/kg/day to about 1 mg/kg/day, about 0.01 mg/kg/day to about 5 mg/kg/day, about 0.01 mg/kg/day to about 10 mg/kg/day, about 0.1 mg/kg/day to about 0.1 mg/kg/day, about 0.1 mg/kg/day to about 0.5 mg/kg/day, about 0.1 mg/kg/day to about 1 mg/kg/day, about 0.1 mg/kg/day to about 5 mg/kg/day, or about 0.1 mg/kg/day to about 10 mg/kg/day.

In aspects of this embodiment, a therapeutically effective amount of a therapeutic compound disclosed herein generally is in the range of about 0.1 mg/day to about 800 mg/day. In other aspects of this embodiment a therapeutically effective amount of a therapeutic compound disclosed herein may be, e.g., at least 0.1 mg/day, at least 0.5 mg/day, at least 1 mg/day, at least 5 mg/day, at least 10 mg/day, at least 20 mg/day, at least 30 mg/day, at least 40 mg/day, at least 50 mg/day, at least 60 mg/day, at least 70 mg/day, at least 80 mg/day, at least 90 mg/day, at least 100 mg/day, at least 150 mg/day, at least 200 mg/day, at least 250 mg/day, at least 300 mg/day, at least 350 mg/day, at least 400 mg/day, at least 450 mg/day, at least 500 mg/day, at least 550 mg/day, at least 600 mg/day, at least 650 mg/day, at least 700 mg/day, at least 750 mg/day, or at least 800 mg/day. In yet other aspects of this embodiment, a therapeutically effective amount of a therapeutic compound disclosed herein may be, e.g., about 0.1 mg/day to about 10 mg/day, about 0.1 mg/day to about 20 mg/day, about 0.1 mg/day to about 40 mg/day, about 0.1 mg/day to about 60 mg/day, about 0.1 mg/day to about 80 mg/day, about 0.1 mg/day to about 100 mg/day, about 1 mg/day to about 10 mg/day, about 1 mg/day to about 20 mg/day, about 1 mg/day to about 40 mg/day, about 1 mg/day to about 60 mg/day, about 1 mg/day to about 80 mg/day, about 1 mg/day to about 100 mg/day, about 1 mg/day to about 150 mg/day, about 1 mg/day to about 200 mg/day, about 1 mg/day to about 250 mg/day, about 1 mg/day to about 300 mg/day, about 1 mg/day to about 350 mg/day, about 1 mg/day to about 400 mg/day, about 1 mg/day to about 450 mg/day, about 1 mg/day to about 500 mg/day, about 1 mg/day to about 550 mg/day, about 1 mg/day to about 600 mg/day, about 1 mg/day to about 650 mg/day, about 1 mg/day to about 700 mg/day, about 1 mg/day to about 750 mg/day, about 1 mg/day to about 800 mg/day, about 2.5 mg/day to about 10 mg/day, about 2.5 mg/day to about 20 mg/day, about 2.5 mg/day to about 40 mg/day, about 2.5 mg/day to about 60 mg/day, about 2.5 mg/day to about 80 mg/day, about 2.5 mg/day to about 100 mg/day, about 2.5 mg/day to about 100 mg/day, about 2.5 mg/day to about 150 mg/day, about 2.5 mg/day to about 200 mg/day, about 2.5 mg/day to about 250 mg/day, about 2.5 mg/day to about 300 mg/day, about 2.5 mg/day to about 350 mg/day, about 2.5 mg/day to about 400 mg/day, about 2.5 mg/day to about 450 mg/day, about 2.5 mg/day to about 500 mg/day, about 2.5 mg/day to about 550 mg/day, about 2.5 mg/day to about 600 mg/day, about 2.5 mg/day to about 650 mg/day, about 2.5 mg/day to about 700 mg/day, about 2.5 mg/day to about 750 mg/day, about 2.5 mg/day to about 800 mg/day, about 10 mg/day to about 100 mg/day, about 10 mg/day to about 150 mg/day, about 10 mg/day to about 200 mg/day, about 10 mg/day to about 250 mg/day, about 10 mg/day to about 300 mg/day, about 10 mg/day to about 350 mg/day, about 10 mg/day to about 400 mg/day, about 10 mg/day to about 450 mg/day, about 10 mg/day to about 500 mg/day, about 10 mg/day to about 550 mg/day, about 10 mg/day to about 600 mg/day, about 10 mg/day to about 650 mg/day, about 10 mg/day to about 700 mg/day, about 10 mg/day to about 750 mg/day, or about 10 mg/day to about 800 mg/day.

Dosing can be single dosage or cumulative (serial dosing), and can be readily determined by one skilled in the art. For instance, treatment of a gender-biased immune disorder may comprise a one-time administration of an effective dose of a pharmaceutical composition disclosed herein. Alternatively, treatment of a gender-biased immune disorder may comprise multiple administrations of an effective dose of a pharmaceutical composition carried out over a range of time periods, such as, e.g., once daily, twice daily, trice daily, once every few days, or once weekly. The timing of administration can vary from individual to individual, depending upon such factors as the severity of an individual's symptoms. For example, an effective dose of a pharmaceutical composition disclosed herein can be administered to an individual once daily for an indefinite period of time, or until the individual no longer requires therapy. A person of ordinary skill in the art will recognize that the condition of the individual can be monitored throughout the course of treatment and that the effective amount of a pharmaceutical composition disclosed herein that is administered can be adjusted accordingly.

Methods of treating and uses pertaining to the compounds and pharmaceutical compositions disclosed herein encompass therapies spanning short periods of time or short duration. This is to ensure that the compounds and pharmaceutical compositions disclosed herein redress a Th1 and/or Th2 response predominance in order to restore a well-balanced immune response, but avoid triggering the biological mechanism regulating the manifestation of secondary sex characteristics in an individual.

In an aspect of this embodiment, a method of treating and use disclosed herein administers a compound and a pharmaceutical composition disclosed herein in an individual in an amount effective to treat a gender-biased immune disorder without resulting in the appearance or manifestation of any secondary sex characteristics in the individual. In an embodiment, a compound and a pharmaceutical composition disclosed herein is administered to an individual for a first period of time followed by a second period of time wherein the compound and/or the pharmaceutical composition disclosed herein is not administered to the individual. In another embodiment, a compound and a pharmaceutical composition disclosed herein is administered to an individual for one or more cycles, wherein the one or more cycles comprises administering a compound and/or a pharmaceutical composition disclosed herein to an individual for a first period of time followed by a second period of time where the compound and/or pharmaceutical composition disclosed herein is not administered to the individual.

In another embodiment, a compound and a pharmaceutical composition disclosed herein is administered to an individual for a plurality of cycles, wherein each of the plurality of cycles comprises administering a compound and/or a pharmaceutical composition disclosed herein to an individual for a first period of time followed by a second period of time where the compound and/or pharmaceutical composition disclosed herein is not administered to the individual. In an aspect of this embodiment, a compound and a pharmaceutical composition disclosed herein is administered to an individual for a plurality of cycles that is maintained for a definite period of time. In another aspect of this embodiment, a compound and a pharmaceutical composition disclosed herein is administered to an individual for a plurality of cycles that is perpetually maintained.

In aspects of this embodiment, a compound and a pharmaceutical composition disclosed herein is administered to an individual for, e.g., about 1 cycles, about 2 cycles, about 3 cycles, about 4 cycles, about 5 cycles, about 6 cycles, about 7 cycles, about 8 cycles, about 9 cycles, about 10 cycles, about 11 cycles, about 12 cycles, about 13 cycles, about 14 cycles, or about 15 cycles. In other aspects of this embodiment, a compound and a pharmaceutical composition disclosed herein is administered to an individual for, e.g., at least one cycle, at least 2 cycles, at least 3 cycles, at least 4 cycles, at least 5 cycles, at least 6 cycles, at least 7 cycles, at least 8 cycles, at least 9 cycles, at least 10 cycles, at least 11 cycles, at least 12 cycles, at least 13 cycles, at least 14 cycles, or at least 15 cycles. In yet other aspects of this embodiment, a compound and a pharmaceutical composition disclosed herein is administered to an individual for, e.g., at most 1 cycle, at most 2 cycles, at most 3 cycles, at most 4 cycles, at most 5 cycles, at most 6 cycles, at most 7 cycles, at most 8 cycles, at most 9 cycles, at most 10 cycles, at most 11 cycles, at most 12 cycles, at most 13 cycles, at most 14 cycles, or at most 15 cycles.

In yet other aspects of this embodiment, a compound and a pharmaceutical composition disclosed herein is administered to an individual for, e.g., about 1 cycle to about 2 cycles, about 1 cycle to about 3 cycles, about 1 cycle to about 4 cycles, about 1 cycle to about 5 cycles, about 1 cycle to about 6 cycles, about 1 cycle to about 7 cycles, about 1 cycle to about 8 cycles, about 1 cycle to about 9 cycles, about 1 cycle to about 10 cycles, about 1 cycle to about 11 cycles, about 1 cycle to about 12 cycles, about 1 cycle to about 13 cycles, about 1 cycle to about 14 cycles, about 1 cycle to about 15 cycles, about 2 cycles to about 3 cycles, about 2 cycles to about 4 cycles, about 2 cycles to about 5 cycles, about 2 cycles to about 6 cycles, about 2 cycles to about 7 cycles, about 2 cycles to about 8 cycles, about 2 cycles to about 9 cycles, about 2 cycles to about 10 cycles, about 2 cycles to about 11 cycles, about 2 cycles to about 12 cycles, about 2 cycles to about 13 cycles, about 2 cycles to about 14 cycles, about 2 cycles to about 15 cycles, about 3 cycles to about 4 cycles, about 3 cycles to about 5 cycles, about 3 cycles to about 6 cycles, about 3 cycles to about 7 cycles, about 3 cycles to about 8 cycles, about 3 cycles to about 9 cycles, about 3 cycles to about 10 cycles, about 3 cycles to about 11 cycles, about 3 cycles to about 12 cycles, about 3 cycles to about 13 cycles, about 3 cycles to about 14 cycles, about 3 cycles to about 15 cycles, about 4 cycles to about 5 cycles, about 4 cycles to about 6 cycles, about 4 cycles to about 7 cycles, about 4 cycles to about 8 cycles, about 4 cycles to about 9 cycles, about 4 cycles to about 10 cycles, about 4 cycles to about 11 cycles, about 4 cycles to about 12 cycles, about 4 cycles to about 13 cycles, about 4 cycles to about 14 cycles, about 4 cycles to about 15 cycles, about 5 cycles to about 6 cycles, about 5 cycles to about 7 cycles, about 5 cycles to about 8 cycles, about 5 cycles to about 9 cycles, about 5 cycles to about 10 cycles, about 5 cycles to about 11 cycles, about 5 cycles to about 12 cycles, about 5 cycles to about 13 cycles, about 5 cycles to about 14 cycles, about 5 cycles to about 15 cycles, about 6 cycles to about 7 cycles, about 6 cycles to about 8 cycles, about 6 cycles to about 9 cycles, about 6 cycles to about 10 cycles, about 6 cycles to about 11 cycles, about 6 cycles to about 12 cycles, about 6 cycles to about 13 cycles, about 6 cycles to about 14 cycles, about 6 cycles to about 15 cycles, about 7 cycles to about 8 cycles, about 7 cycles to about 9 cycles, about 7 cycles to about 10 cycles, about 7 cycles to about 11 cycles, about 7 cycles to about 12 cycles, about 7 cycles to about 13 cycles, about 7 cycles to about 14 cycles, about 7 cycles to about 15 cycles, about 8 cycles to about 9 cycles, about 8 cycles to about 10 cycles, about 8 cycles to about 11 cycles, about 8 cycles to about 12 cycles, about 8 cycles to about 13 cycles, about 8 cycles to about 14 cycles, about 8 cycles to about 15 cycles, about 9 cycles to about 10 cycles, about 9 cycles to about 11 cycles, about 9 cycles to about 12 cycles, about 9 cycles to about 13 cycles, about 9 cycles to about 14 cycles, about 9 cycles to about 15 cycles, about 10 cycles to about 11 cycles, about 10 cycles to about 12 cycles, about 10 cycles to about 13 cycles, about 10 cycles to about 14 cycles, about 10 cycles to about 15 cycles, about 11 cycles to about 12 cycles, about 11 cycles to about 13 cycles, about 11 cycles to about 14 cycles, about 11 cycles to about 15 cycles, about 12 cycles to about 13 cycles, about 12 cycles to about 14 cycles, about 12 cycles to about 15 cycles, about 13 cycles to about 14 cycles, about 13 cycles to about 15 cycles, or about 14 cycles to about 15 cycles.

A first period of time during which a compound and/or a pharmaceutical composition disclosed herein may be administered to an individual may be of any duration so long as the appearance or manifestation of any secondary sex characteristics in an individual does not occur. In aspects of this embodiment, a first period of time during which a compound and/or a pharmaceutical composition disclosed herein may administered to an individual may be for, e.g., about 1 day, about 2 days, about 3 days, about 4 days, about 5 days, about 6 days, about 7 days, about 8 days, about 9 days, about 10 days, about 11 days, about 12 days, about 13 days, about 14 days, about 3 weeks, about 4 weeks, about 5 weeks, about 6 weeks, about 7 weeks, about 8 weeks, about 9 weeks, about 10 weeks, about 11 weeks, or about 12 weeks. In other aspects of this embodiment, a first period of time during which a compound and/or a pharmaceutical composition disclosed herein may administered to an individual may be for, e.g., at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 3 weeks, at least 4 weeks, at least 5 weeks, at least 6 weeks, at least 7 weeks, at least 8 weeks, at least 9 weeks, at least 10 weeks, at least 11 weeks, or at least 12 weeks. In yet other aspects of this embodiment, a first period of time during which a compound and/or a pharmaceutical composition disclosed herein may administered to an individual may be for, e.g., at most 1 day, at most 2 days, at most 3 days, at most 4 days, at most 5 days, at most 6 days, at most 7 days, at most 8 days, at most 9 days, at most 10 days, at most 11 days, at most 12 days, at most 13 days, at most 14 days, at most 3 weeks, at most 4 weeks, at most 5 weeks, at most 6 weeks, at most 7 weeks, at most 8 weeks, at most 9 weeks, at most 10 weeks, at most 11 weeks, or at most 12 weeks.

In still other aspects of this embodiment, a first period of time during which a compound and/or a pharmaceutical composition disclosed herein may administered to an individual may be for, e.g., about 1 day to about 3 days, about 1 day to about 5 days, about 1 day to about 7 days, about 1 day to about 9 days, about 1 day to about 10 days, about 1 day to about 12 days, about 1 day to about 14 days, about 1 day to about 15 days, about 1 day to about 18 days, about 1 day to about 21 days, about 1 day to about 24 days, about 1 day to about 27 days, about 1 day to about 28 days, about 3 days to about 5 days, about 3 days to about 7 days, about 3 days to about 9 days, about 3 days to about 10 days, about 3 days to about 12 days, about 3 days to about 14 days, about 3 days to about 15 days, about 3 days to about 18 days, about 3 days to about 21 days, about 3 days to about 24 days, about 3 days to about 27 days, about 3 days to about 28 days, about 5 days to about 7 days, about 5 days to about 9 days, about 5 days to about 10 days, about 5 days to about 12 days, about 5 days to about 14 days, about 5 days to about 15 days, about 5 days to about 18 days, about 5 days to about 21 days, about 5 days to about 24 days, about 5 days to about 27 days, about 5 days to about 28 days, about 7 days to about 9 days, about 7 days to about 10 days, about 7 days to about 12 days, about 7 days to about 14 days, about 7 days to about 17 days, about 7 days to about 18 days, about 7 days to about 21 days, about 7 days to about 24 days, about 7 days to about 27 days, about 7 days to about 28 days, about 10 days to about 12 days, about 10 days to about 14 days, about 10 days to about 17 days, about 10 days to about 18 days, about 10 days to about 21 days, about 10 days to about 24 days, about 10 days to about 27 days, or about 10 days to about 28 days.

In still other aspects of this embodiment, a first period of time during which a compound and/or a pharmaceutical composition disclosed herein may administered to an individual may be for, e.g., about 1 week to about 2 weeks, about 1 week to about 3 weeks, about 1 week to about 4 weeks, about 1 week to about 5 weeks, about 1 week to about 6 weeks, about 1 week to about 7 weeks, about 1 week to about 8 weeks, about 1 week to about 9 weeks, about 1 week to about 10 weeks, about 1 week to about 11 weeks, about 1 week to about 12 weeks, about 2 weeks to about 3 weeks, about 2 weeks to about 4 weeks, about 2 weeks to about 5 weeks, about 2 weeks to about 6 weeks, about 2 weeks to about 7 weeks, about 2 weeks to about 8 weeks, about 2 weeks to about 9 weeks, about 2 weeks to about 10 weeks, about 2 weeks to about 11 weeks, about 2 weeks to about 12 weeks, about 3 weeks to about 4 weeks, about 3 weeks to about 5 weeks, about 3 weeks to about 6 weeks, about 3 weeks to about 7 weeks, about 3 weeks to about 8 weeks, about 3 weeks to about 9 weeks, about 3 weeks to about 10 weeks, about 3 weeks to about 11 weeks, about 3 weeks to about 12 weeks, about 4 weeks to about 5 weeks, about 4 weeks to about 6 weeks, about 4 weeks to about 7 weeks, about 4 weeks to about 8 weeks, about 4 weeks to about 9 weeks, about 4 weeks to about 10 weeks, about 4 weeks to about 11 weeks, about 4 weeks to about 12 weeks, about 5 weeks to about 6 weeks, about 5 weeks to about 7 weeks, about 5 weeks to about 8 weeks, about 5 weeks to about 9 weeks, about 5 weeks to about 10 weeks, about 5 weeks to about 11 weeks, about 5 weeks to about 12 weeks, about 6 weeks to about 7 weeks, about 6 weeks to about 8 weeks, about 6 weeks to about 9 weeks, about 6 weeks to about 10 weeks, about 6 weeks to about 11 weeks, about 6 weeks to about 12 weeks, about 7 weeks to about 8 weeks, about 7 weeks to about 9 weeks, about 7 weeks to about 10 weeks, about 7 weeks to about 11 weeks, about 7 weeks to about 12 weeks, about 8 weeks to about 9 weeks, about 8 weeks to about 10 weeks, about 8 weeks to about 11 weeks, about 8 weeks to about 12 weeks, about 9 weeks to about 10 weeks, about 9 weeks to about 11 weeks, about 9 weeks to about 12 weeks, about 10 weeks to about 11 weeks, about 10 weeks to about 12 weeks, or about 11 weeks to about 12 weeks.

A second period of time during which a compound and/or a pharmaceutical composition disclosed herein may not be administered to an individual may be of any duration. In aspects of this embodiment, a second period of time during which a compound and/or a pharmaceutical composition disclosed herein may not be administered to an individual may be for, e.g., about 1 day, about 2 days, about 3 days, about 4 days, about 5 days, about 6 days, about 7 days, about 8 days, about 9 days, about 10 days, about 11 days, about 12 days, about 13 days, about 14 days, about 3 weeks, about 4 weeks, about 5 weeks, about 6 weeks, about 7 weeks, about 8 weeks, about 9 weeks, about 10 weeks, about 11 weeks, about 12 weeks, about 1 month, about 2 months, about 3 months, about 4 months, about 5 months, about 6 months, about 7 months, about 8 months, about 9 months, about 10 months, about 11 months, or about 12 months. In other aspects of this embodiment, a second period of time during which a compound and/or a pharmaceutical composition disclosed herein may not be administered to an individual may be for, e.g., at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 1 month, at least 2 months, at least 3 months, at least 4 months, at least 5 months, at least 6 months, at least 7 months, at least 8 months, at least 9 months, at least 10 months, at least 11 months, or at least 12 months. In yet other aspects of this embodiment, a first period of time during which a compound and/or a pharmaceutical composition disclosed herein may not be administered to an individual may be for, e.g., at most 1 day, at most 2 days, at most 3 days, at most 4 days, at most 5 days, at most 6 days, at most 7 days, at most 8 days, at most 9 days, at most 10 days, at most 11 days, at most 12 days, at most 13 days, at most 14 days, at most 3 weeks, at most 4 weeks, at most 5 weeks, at most 6 weeks, at most 7 weeks, at most 8 weeks, at most 9 weeks, at most 10 weeks, at most 11 weeks, at most 12 weeks, at most 1 month, at most 2 months, at most 3 months, at most 4 months, at most 5 months, at most 6 months, at most 7 months, at most 8 months, at most 9 months, at most 10 months, at most 11 months, or at most 12 months.

In still other aspects of this embodiment, a second period of time during which a compound and/or a pharmaceutical composition disclosed herein may not be administered to an individual may be for, e.g., about 1 day to about 3 days, about 1 day to about 5 days, about 1 day to about 7 days, about 1 day to about 9 days, about 1 day to about 10 days, about 1 day to about 12 days, about 1 day to about 14 days, about 1 day to about 15 days, about 1 day to about 18 days, about 1 day to about 21 days, about 1 day to about 24 days, about 1 day to about 27 days, about 1 day to about 28 days, about 3 days to about 5 days, about 3 days to about 7 days, about 3 days to about 9 days, about 3 days to about 10 days, about 3 days to about 12 days, about 3 days to about 14 days, about 3 days to about 15 days, about 3 days to about 18 days, about 3 days to about 21 days, about 3 days to about 24 days, about 3 days to about 27 days, about 3 days to about 28 days, about 5 days to about 7 days, about 5 days to about 9 days, about 5 days to about 10 days, about 5 days to about 12 days, about 5 days to about 14 days, about 5 days to about 15 days, about 5 days to about 18 days, about 5 days to about 21 days, about 5 days to about 24 days, about 5 days to about 27 days, about 5 days to about 28 days, about 7 days to about 9 days, about 7 days to about 10 days, about 7 days to about 12 days, about 7 days to about 14 days, about 7 days to about 17 days, about 7 days to about 18 days, about 7 days to about 21 days, about 7 days to about 24 days, about 7 days to about 27 days, about 7 days to about 28 days, about 10 days to about 12 days, about 10 days to about 14 days, about 10 days to about 17 days, about 10 days to about 18 days, about 10 days to about 21 days, about 10 days to about 24 days, about 10 days to about 27 days, or about 10 days to about 28 days.

In still other aspects of this embodiment, a second period of time during which a compound and/or a pharmaceutical composition disclosed herein may not be administered to an individual may be for, e.g., about 1 week to about 2 weeks, about 1 week to about 3 weeks, about 1 week to about 4 weeks, about 1 week to about 5 weeks, about 1 week to about 6 weeks, about 1 week to about 7 weeks, about 1 week to about 8 weeks, about 1 week to about 9 weeks, about 1 week to about 10 weeks, about 1 week to about 11 weeks, about 1 week to about 12 weeks, about 2 weeks to about 3 weeks, about 2 weeks to about 4 weeks, about 2 weeks to about 5 weeks, about 2 weeks to about 6 weeks, about 2 weeks to about 7 weeks, about 2 weeks to about 8 weeks, about 2 weeks to about 9 weeks, about 2 weeks to about 10 weeks, about 2 weeks to about 11 weeks, about 2 weeks to about 12 weeks, about 3 weeks to about 4 weeks, about 3 weeks to about 5 weeks, about 3 weeks to about 6 weeks, about 3 weeks to about 7 weeks, about 3 weeks to about 8 weeks, about 3 weeks to about 9 weeks, about 3 weeks to about 10 weeks, about 3 weeks to about 11 weeks, about 3 weeks to about 12 weeks, about 4 weeks to about 5 weeks, about 4 weeks to about 6 weeks, about 4 weeks to about 7 weeks, about 4 weeks to about 8 weeks, about 4 weeks to about 9 weeks, about 4 weeks to about 10 weeks, about 4 weeks to about 11 weeks, about 4 weeks to about 12 weeks, about 5 weeks to about 6 weeks, about 5 weeks to about 7 weeks, about 5 weeks to about 8 weeks, about 5 weeks to about 9 weeks, about 5 weeks to about 10 weeks, about 5 weeks to about 11 weeks, about 5 weeks to about 12 weeks, about 6 weeks to about 7 weeks, about 6 weeks to about 8 weeks, about 6 weeks to about 9 weeks, about 6 weeks to about 10 weeks, about 6 weeks to about 11 weeks, about 6 weeks to about 12 weeks, about 7 weeks to about 8 weeks, about 7 weeks to about 9 weeks, about 7 weeks to about 10 weeks, about 7 weeks to about 11 weeks, about 7 weeks to about 12 weeks, about 8 weeks to about 9 weeks, about 8 weeks to about 10 weeks, about 8 weeks to about 11 weeks, about 8 weeks to about 12 weeks, about 9 weeks to about 10 weeks, about 9 weeks to about 11 weeks, about 9 weeks to about 12 weeks, about 10 weeks to about 11 weeks, about 10 weeks to about 12 weeks, or about 11 weeks to about 12 weeks.

In still other aspects of this embodiment, a second period of time during which a compound and/or a pharmaceutical composition disclosed herein may not be administered to an individual may be for, e.g., about 1 month to about 2 months, about 1 month to about 3 months, about 1 month to about 4 months, about 1 month to about 5 months, about 1 month to about 6 months, about 1 month to about 7 months, about 1 month to about 8 months, about 1 month to about 9 months, about 1 month to about 10 months, about 1 month to about 11 months, about 1 month to about 12 months, about 2 months to about 3 months, about 2 months to about 4 months, about 2 months to about 5 months, about 2 months to about 6 months, about 2 months to about 7 months, about 2 months to about 8 months, about 2 months to about 9 months, about 2 months to about 10 months, about 2 months to about 11 months, about 2 months to about 12 months, about 3 months to about 4 months, about 3 months to about 5 months, about 3 months to about 6 months, about 3 months to about 7 months, about 3 months to about 8 months, about 3 months to about 9 months, about 3 months to about 10 months, about 3 months to about 11 months, about 3 months to about 12 months, about 4 months to about 5 months, about 4 months to about 6 months, about 4 months to about 7 months, about 4 months to about 8 months, about 4 months to about 9 months, about 4 months to about 10 months, about 4 months to about 11 months, about 4 months to about 12 months, about 5 months to about 6 months, about 5 months to about 7 months, about 5 months to about 8 months, about 5 months to about 9 months, about 5 months to about 10 months, about 5 months to about 11 months, about 5 months to about 12 months, about 6 months to about 7 months, about 6 months to about 8 months, about 6 months to about 9 months, about 6 months to about 10 months, about 6 months to about 11 months, about 6 months to about 12 months, about 7 months to about 8 months, about 7 months to about 9 months, about 7 months to about 10 months, about 7 months to about 11 months, about 7 months to about 12 months, about 8 months to about 9 months, about 8 months to about 10 months, about 8 months to about 11 months, about 8 months to about 12 months, about 9 months to about 10 months, about 9 months to about 11 months, about 9 months to about 12 months, about 10 months to about 11 months, about 10 months to about 12 months, or about 11 months to about 12 months.

In other aspects of this embodiment, a first period of time during which a compound and/or a pharmaceutical composition disclosed herein may administered to an individual may be for, e.g., about 1 day, about 2 days, about 3 days, about 4 days, about 5 days, about 6 days, about 7 days, about 8 days, about 9 days, about 10 days, about 11 days, about 12 days, about 13 days, about 14 days, about 3 weeks, about 4 weeks, about 5 weeks, about 6 weeks, about 7 weeks, about 8 weeks, about 9 weeks, about 10 weeks, about 11 weeks, or about 12 weeks and a second period of time during which a compound and/or a pharmaceutical composition disclosed herein may not be administered to an individual may be for, e.g., about 1 day, about 2 days, about 3 days, about 4 days, about 5 days, about 6 days, about 7 days, about 8 days, about 9 days, about 10 days, about 11 days, about 12 days, about 13 days, about 14 days, about 3 weeks, about 4 weeks, about 5 weeks, about 6 weeks, about 7 weeks, about 8 weeks, about 9 weeks, about 10 weeks, about 11 weeks, about 12 weeks, about 1 month, about 2 months, about 3 months, about 4 months, about 5 months, about 6 months, about 7 months, about 8 months, about 9 months, about 10 months, about 11 months, about 12 months, at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at most 1 day, at most 2 days, at most 3 days, at most 4 days, at most 5 days, at most 6 days, at most 7 days, at most 8 days, at most 9 days, at most 10 days, at most 11 days, at most 12 days, at most 13 days, at most 14 days, at most 3 weeks, at most 4 weeks, at most 5 weeks, at most 6 weeks, at most 7 weeks, at most 8 weeks, at most 9 weeks, at most 10 weeks, at most 11 weeks, at most 12 weeks, at most 1 month, at most 2 months, at most 3 months, at most 4 months, at most 5 months, at most 6 months, at most 7 months, at most 8 months, at most 9 months, at most 10 months, at most 11 months, or at most 12 months, about 1 day to about 3 days, about 1 day to about 5 days, about 1 day to about 7 days, about 1 day to about 9 days, about 1 day to about 10 days, about 1 day to about 12 days, about 1 day to about 14 days, about 1 day to about 15 days, about 1 day to about 18 days, about 1 day to about 21 days, about 1 day to about 24 days, about 1 day to about 27 days, about 1 day to about 28 days, about 3 days to about 5 days, about 3 days to about 7 days, about 3 days to about 9 days, about 3 days to about 10 days, about 3 days to about 12 days, about 3 days to about 14 days, about 3 days to about 15 days, about 3 days to about 18 days, about 3 days to about 21 days, about 3 days to about 24 days, about 3 days to about 27 days, about 3 days to about 28 days, about 5 days to about 7 days, about 5 days to about 9 days, about 5 days to about 10 days, about 5 days to about 12 days, about 5 days to about 14 days, about 5 days to about 15 days, about 5 days to about 18 days, about 5 days to about 21 days, about 5 days to about 24 days, about 5 days to about 27 days, about 5 days to about 28 days, about 7 days to about 9 days, about 7 days to about 10 days, about 7 days to about 12 days, about 7 days to about 14 days, about 7 days to about 17 days, about 7 days to about 18 days, about 7 days to about 21 days, about 7 days to about 24 days, about 7 days to about 27 days, about 7 days to about 28 days, about 10 days to about 12 days, about 10 days to about 14 days, about 10 days to about 17 days, about 10 days to about 18 days, about 10 days to about 21 days, about 10 days to about 24 days, about 10 days to about 27 days, about 10 days to about 28 days, about 1 week to about 2 weeks, about 1 week to about 3 weeks, about 1 week to about 4 weeks, about 1 week to about 5 weeks, about 1 week to about 6 weeks, about 1 week to about 7 weeks, about 1 week to about 8 weeks, about 1 week to about 9 weeks, about 1 week to about 10 weeks, about 1 week to about 11 weeks, about 1 week to about 12 weeks, about 2 weeks to about 3 weeks, about 2 weeks to about 4 weeks, about 2 weeks to about 5 weeks, about 2 weeks to about 6 weeks, about 2 weeks to about 7 weeks, about 2 weeks to about 8 weeks, about 2 weeks to about 9 weeks, about 2 weeks to about 10 weeks, about 2 weeks to about 11 weeks, about 2 weeks to about 12 weeks, about 3 weeks to about 4 weeks, about 3 weeks to about 5 weeks, about 3 weeks to about 6 weeks, about 3 weeks to about 7 weeks, about 3 weeks to about 8 weeks, about 3 weeks to about 9 weeks, about 3 weeks to about 10 weeks, about 3 weeks to about 11 weeks, about 3 weeks to about 12 weeks, about 4 weeks to about 5 weeks, about 4 weeks to about 6 weeks, about 4 weeks to about 7 weeks, about 4 weeks to about 8 weeks, about 4 weeks to about 9 weeks, about 4 weeks to about 10 weeks, about 4 weeks to about 11 weeks, about 4 weeks to about 12 weeks, about 5 weeks to about 6 weeks, about 5 weeks to about 7 weeks, about 5 weeks to about 8 weeks, about 5 weeks to about 9 weeks, about 5 weeks to about 10 weeks, about 5 weeks to about 11 weeks, about 5 weeks to about 12 weeks, about 6 weeks to about 7 weeks, about 6 weeks to about 8 weeks, about 6 weeks to about 9 weeks, about 6 weeks to about 10 weeks, about 6 weeks to about 11 weeks, about 6 weeks to about 12 weeks, about 7 weeks to about 8 weeks, about 7 weeks to about 9 weeks, about 7 weeks to about 10 weeks, about 7 weeks to about 11 weeks, about 7 weeks to about 12 weeks, about 8 weeks to about 9 weeks, about 8 weeks to about 10 weeks, about 8 weeks to about 11 weeks, about 8 weeks to about 12 weeks, about 9 weeks to about 10 weeks, about 9 weeks to about 11 weeks, about 9 weeks to about 12 weeks, about 10 weeks to about 11 weeks, about 10 weeks to about 12 weeks, about 11 weeks to about 12 weeks, about 1 month to about 2 months, about 1 month to about 3 months, about 1 month to about 4 months, about 1 month to about 5 months, about 1 month to about 6 months, about 1 month to about 7 months, about 1 month to about 8 months, about 1 month to about 9 months, about 1 month to about 10 months, about 1 month to about 11 months, about 1 month to about 12 months, about 2 months to about 3 months, about 2 months to about 4 months, about 2 months to about 5 months, about 2 months to about 6 months, about 2 months to about 7 months, about 2 months to about 8 months, about 2 months to about 9 months, about 2 months to about 10 months, about 2 months to about 11 months, about 2 months to about 12 months, about 3 months to about 4 months, about 3 months to about 5 months, about 3 months to about 6 months, about 3 months to about 7 months, about 3 months to about 8 months, about 3 months to about 9 months, about 3 months to about 10 months, about 3 months to about 11 months, about 3 months to about 12 months, about 4 months to about 5 months, about 4 months to about 6 months, about 4 months to about 7 months, about 4 months to about 8 months, about 4 months to about 9 months, about 4 months to about 10 months, about 4 months to about 11 months, about 4 months to about 12 months, about 5 months to about 6 months, about 5 months to about 7 months, about 5 months to about 8 months, about 5 months to about 9 months, about 5 months to about 10 months, about 5 months to about 11 months, about 5 months to about 12 months, about 6 months to about 7 months, about 6 months to about 8 months, about 6 months to about 9 months, about 6 months to about 10 months, about 6 months to about 11 months, about 6 months to about 12 months, about 7 months to about 8 months, about 7 months to about 9 months, about 7 months to about 10 months, about 7 months to about 11 months, about 7 months to about 12 months, about 8 months to about 9 months, about 8 months to about 10 months, about 8 months to about 11 months, about 8 months to about 12 months, about 9 months to about 10 months, about 9 months to about 11 months, about 9 months to about 12 months, about 10 months to about 11 months, about 10 months to about 12 months, or about 11 months to about 12 months.

In other aspects of this embodiment, a first period of time during which a compound and/or a pharmaceutical composition disclosed herein may administered to an individual may be for, e.g., at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 3 weeks, at least 4 weeks, at least 5 weeks, at least 6 weeks, at least 7 weeks, at least 8 weeks, at least 9 weeks, at least 10 weeks, at least 11 weeks, or at least 12 weeks, and a second period of time during which a compound and/or a pharmaceutical composition disclosed herein may not be administered to an individual may be for, e.g., about 1 day, about 2 days, about 3 days, about 4 days, about 5 days, about 6 days, about 7 days, about 8 days, about 9 days, about 10 days, about 11 days, about 12 days, about 13 days, about 14 days, about 3 weeks, about 4 weeks, about 5 weeks, about 6 weeks, about 7 weeks, about 8 weeks, about 9 weeks, about 10 weeks, about 11 weeks, about 12 weeks, about 1 month, about 2 months, about 3 months, about 4 months, about 5 months, about 6 months, about 7 months, about 8 months, about 9 months, about 10 months, about 11 months, about 12 months, at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at most 1 day, at most 2 days, at most 3 days, at most 4 days, at most 5 days, at most 6 days, at most 7 days, at most 8 days, at most 9 days, at most 10 days, at most 11 days, at most 12 days, at most 13 days, at most 14 days, at most 3 weeks, at most 4 weeks, at most 5 weeks, at most 6 weeks, at most 7 weeks, at most 8 weeks, at most 9 weeks, at most 10 weeks, at most 11 weeks, at most 12 weeks, at most 1 month, at most 2 months, at most 3 months, at most 4 months, at most 5 months, at most 6 months, at most 7 months, at most 8 months, at most 9 months, at most 10 months, at most 11 months, or at most 12 months, about 1 day to about 3 days, about 1 day to about 5 days, about 1 day to about 7 days, about 1 day to about 9 days, about 1 day to about 10 days, about 1 day to about 12 days, about 1 day to about 14 days, about 1 day to about 15 days, about 1 day to about 18 days, about 1 day to about 21 days, about 1 day to about 24 days, about 1 day to about 27 days, about 1 day to about 28 days, about 3 days to about 5 days, about 3 days to about 7 days, about 3 days to about 9 days, about 3 days to about 10 days, about 3 days to about 12 days, about 3 days to about 14 days, about 3 days to about 15 days, about 3 days to about 18 days, about 3 days to about 21 days, about 3 days to about 24 days, about 3 days to about 27 days, about 3 days to about 28 days, about 5 days to about 7 days, about 5 days to about 9 days, about 5 days to about 10 days, about 5 days to about 12 days, about 5 days to about 14 days, about 5 days to about 15 days, about 5 days to about 18 days, about 5 days to about 21 days, about 5 days to about 24 days, about 5 days to about 27 days, about 5 days to about 28 days, about 7 days to about 9 days, about 7 days to about 10 days, about 7 days to about 12 days, about 7 days to about 14 days, about 7 days to about 17 days, about 7 days to about 18 days, about 7 days to about 21 days, about 7 days to about 24 days, about 7 days to about 27 days, about 7 days to about 28 days, about 10 days to about 12 days, about 10 days to about 14 days, about 10 days to about 17 days, about 10 days to about 18 days, about 10 days to about 21 days, about 10 days to about 24 days, about 10 days to about 27 days, about 10 days to about 28 days, about 1 week to about 2 weeks, about 1 week to about 3 weeks, about 1 week to about 4 weeks, about 1 week to about 5 weeks, about 1 week to about 6 weeks, about 1 week to about 7 weeks, about 1 week to about 8 weeks, about 1 week to about 9 weeks, about 1 week to about 10 weeks, about 1 week to about 11 weeks, about 1 week to about 12 weeks, about 2 weeks to about 3 weeks, about 2 weeks to about 4 weeks, about 2 weeks to about 5 weeks, about 2 weeks to about 6 weeks, about 2 weeks to about 7 weeks, about 2 weeks to about 8 weeks, about 2 weeks to about 9 weeks, about 2 weeks to about 10 weeks, about 2 weeks to about 11 weeks, about 2 weeks to about 12 weeks, about 3 weeks to about 4 weeks, about 3 weeks to about 5 weeks, about 3 weeks to about 6 weeks, about 3 weeks to about 7 weeks, about 3 weeks to about 8 weeks, about 3 weeks to about 9 weeks, about 3 weeks to about 10 weeks, about 3 weeks to about 11 weeks, about 3 weeks to about 12 weeks, about 4 weeks to about 5 weeks, about 4 weeks to about 6 weeks, about 4 weeks to about 7 weeks, about 4 weeks to about 8 weeks, about 4 weeks to about 9 weeks, about 4 weeks to about 10 weeks, about 4 weeks to about 11 weeks, about 4 weeks to about 12 weeks, about 5 weeks to about 6 weeks, about 5 weeks to about 7 weeks, about 5 weeks to about 8 weeks, about 5 weeks to about 9 weeks, about 5 weeks to about 10 weeks, about 5 weeks to about 11 weeks, about 5 weeks to about 12 weeks, about 6 weeks to about 7 weeks, about 6 weeks to about 8 weeks, about 6 weeks to about 9 weeks, about 6 weeks to about 10 weeks, about 6 weeks to about 11 weeks, about 6 weeks to about 12 weeks, about 7 weeks to about 8 weeks, about 7 weeks to about 9 weeks, about 7 weeks to about 10 weeks, about 7 weeks to about 11 weeks, about 7 weeks to about 12 weeks, about 8 weeks to about 9 weeks, about 8 weeks to about 10 weeks, about 8 weeks to about 11 weeks, about 8 weeks to about 12 weeks, about 9 weeks to about 10 weeks, about 9 weeks to about 11 weeks, about 9 weeks to about 12 weeks, about 10 weeks to about 11 weeks, about 10 weeks to about 12 weeks, about 11 weeks to about 12 weeks, about 1 month to about 2 months, about 1 month to about 3 months, about 1 month to about 4 months, about 1 month to about 5 months, about 1 month to about 6 months, about 1 month to about 7 months, about 1 month to about 8 months, about 1 month to about 9 months, about 1 month to about 10 months, about 1 month to about 11 months, about 1 month to about 12 months, about 2 months to about 3 months, about 2 months to about 4 months, about 2 months to about 5 months, about 2 months to about 6 months, about 2 months to about 7 months, about 2 months to about 8 months, about 2 months to about 9 months, about 2 months to about 10 months, about 2 months to about 11 months, about 2 months to about 12 months, about 3 months to about 4 months, about 3 months to about 5 months, about 3 months to about 6 months, about 3 months to about 7 months, about 3 months to about 8 months, about 3 months to about 9 months, about 3 months to about 10 months, about 3 months to about 11 months, about 3 months to about 12 months, about 4 months to about 5 months, about 4 months to about 6 months, about 4 months to about 7 months, about 4 months to about 8 months, about 4 months to about 9 months, about 4 months to about 10 months, about 4 months to about 11 months, about 4 months to about 12 months, about 5 months to about 6 months, about 5 months to about 7 months, about 5 months to about 8 months, about 5 months to about 9 months, about 5 months to about 10 months, about 5 months to about 11 months, about 5 months to about 12 months, about 6 months to about 7 months, about 6 months to about 8 months, about 6 months to about 9 months, about 6 months to about 10 months, about 6 months to about 11 months, about 6 months to about 12 months, about 7 months to about 8 months, about 7 months to about 9 months, about 7 months to about 10 months, about 7 months to about 11 months, about 7 months to about 12 months, about 8 months to about 9 months, about 8 months to about 10 months, about 8 months to about 11 months, about 8 months to about 12 months, about 9 months to about 10 months, about 9 months to about 11 months, about 9 months to about 12 months, about 10 months to about 11 months, about 10 months to about 12 months, or about 11 months to about 12 months.

In yet other aspects of this embodiment, a first period of time during which a compound and/or a pharmaceutical composition disclosed herein may administered to an individual may be for, e.g., at most 1 day, at most 2 days, at most 3 days, at most 4 days, at most 5 days, at most 6 days, at most 7 days, at most 8 days, at most 9 days, at most 10 days, at most 11 days, at most 12 days, at most 13 days, at most 14 days, at most 3 weeks, at most 4 weeks, at most 5 weeks, at most 6 weeks, at most 7 weeks, at most 8 weeks, at most 9 weeks, at most 10 weeks, at most 11 weeks, or at most 12 weeks, and a second period of time during which a compound and/or a pharmaceutical composition disclosed herein may not be administered to an individual may be for, e.g., about 1 day, about 2 days, about 3 days, about 4 days, about 5 days, about 6 days, about 7 days, about 8 days, about 9 days, about 10 days, about 11 days, about 12 days, about 13 days, about 14 days, about 3 weeks, about 4 weeks, about 5 weeks, about 6 weeks, about 7 weeks, about 8 weeks, about 9 weeks, about 10 weeks, about 11 weeks, about 12 weeks, about 1 month, about 2 months, about 3 months, about 4 months, about 5 months, about 6 months, about 7 months, about 8 months, about 9 months, about 10 months, about 11 months, about 12 months, at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at most 1 day, at most 2 days, at most 3 days, at most 4 days, at most 5 days, at most 6 days, at most 7 days, at most 8 days, at most 9 days, at most 10 days, at most 11 days, at most 12 days, at most 13 days, at most 14 days, at most 3 weeks, at most 4 weeks, at most 5 weeks, at most 6 weeks, at most 7 weeks, at most 8 weeks, at most 9 weeks, at most 10 weeks, at most 11 weeks, at most 12 weeks, at most 1 month, at most 2 months, at most 3 months, at most 4 months, at most 5 months, at most 6 months, at most 7 months, at most 8 months, at most 9 months, at most 10 months, at most 11 months, or at most 12 months, about 1 day to about 3 days, about 1 day to about 5 days, about 1 day to about 7 days, about 1 day to about 9 days, about 1 day to about 10 days, about 1 day to about 12 days, about 1 day to about 14 days, about 1 day to about 15 days, about 1 day to about 18 days, about 1 day to about 21 days, about 1 day to about 24 days, about 1 day to about 27 days, about 1 day to about 28 days, about 3 days to about 5 days, about 3 days to about 7 days, about 3 days to about 9 days, about 3 days to about 10 days, about 3 days to about 12 days, about 3 days to about 14 days, about 3 days to about 15 days, about 3 days to about 18 days, about 3 days to about 21 days, about 3 days to about 24 days, about 3 days to about 27 days, about 3 days to about 28 days, about 5 days to about 7 days, about 5 days to about 9 days, about 5 days to about 10 days, about 5 days to about 12 days, about 5 days to about 14 days, about 5 days to about 15 days, about 5 days to about 18 days, about 5 days to about 21 days, about 5 days to about 24 days, about 5 days to about 27 days, about 5 days to about 28 days, about 7 days to about 9 days, about 7 days to about 10 days, about 7 days to about 12 days, about 7 days to about 14 days, about 7 days to about 17 days, about 7 days to about 18 days, about 7 days to about 21 days, about 7 days to about 24 days, about 7 days to about 27 days, about 7 days to about 28 days, about 10 days to about 12 days, about 10 days to about 14 days, about 10 days to about 17 days, about 10 days to about 18 days, about 10 days to about 21 days, about 10 days to about 24 days, about 10 days to about 27 days, about 10 days to about 28 days, about 1 week to about 2 weeks, about 1 week to about 3 weeks, about 1 week to about 4 weeks, about 1 week to about 5 weeks, about 1 week to about 6 weeks, about 1 week to about 7 weeks, about 1 week to about 8 weeks, about 1 week to about 9 weeks, about 1 week to about 10 weeks, about 1 week to about 11 weeks, about 1 week to about 12 weeks, about 2 weeks to about 3 weeks, about 2 weeks to about 4 weeks, about 2 weeks to about 5 weeks, about 2 weeks to about 6 weeks, about 2 weeks to about 7 weeks, about 2 weeks to about 8 weeks, about 2 weeks to about 9 weeks, about 2 weeks to about 10 weeks, about 2 weeks to about 11 weeks, about 2 weeks to about 12 weeks, about 3 weeks to about 4 weeks, about 3 weeks to about 5 weeks, about 3 weeks to about 6 weeks, about 3 weeks to about 7 weeks, about 3 weeks to about 8 weeks, about 3 weeks to about 9 weeks, about 3 weeks to about 10 weeks, about 3 weeks to about 11 weeks, about 3 weeks to about 12 weeks, about 4 weeks to about 5 weeks, about 4 weeks to about 6 weeks, about 4 weeks to about 7 weeks, about 4 weeks to about 8 weeks, about 4 weeks to about 9 weeks, about 4 weeks to about 10 weeks, about 4 weeks to about 11 weeks, about 4 weeks to about 12 weeks, about 5 weeks to about 6 weeks, about 5 weeks to about 7 weeks, about 5 weeks to about 8 weeks, about 5 weeks to about 9 weeks, about 5 weeks to about 10 weeks, about 5 weeks to about 11 weeks, about 5 weeks to about 12 weeks, about 6 weeks to about 7 weeks, about 6 weeks to about 8 weeks, about 6 weeks to about 9 weeks, about 6 weeks to about 10 weeks, about 6 weeks to about 11 weeks, about 6 weeks to about 12 weeks, about 7 weeks to about 8 weeks, about 7 weeks to about 9 weeks, about 7 weeks to about 10 weeks, about 7 weeks to about 11 weeks, about 7 weeks to about 12 weeks, about 8 weeks to about 9 weeks, about 8 weeks to about 10 weeks, about 8 weeks to about 11 weeks, about 8 weeks to about 12 weeks, about 9 weeks to about 10 weeks, about 9 weeks to about 11 weeks, about 9 weeks to about 12 weeks, about 10 weeks to about 11 weeks, about 10 weeks to about 12 weeks, about 11 weeks to about 12 weeks, about 1 month to about 2 months, about 1 month to about 3 months, about 1 month to about 4 months, about 1 month to about 5 months, about 1 month to about 6 months, about 1 month to about 7 months, about 1 month to about 8 months, about 1 month to about 9 months, about 1 month to about 10 months, about 1 month to about 11 months, about 1 month to about 12 months, about 2 months to about 3 months, about 2 months to about 4 months, about 2 months to about 5 months, about 2 months to about 6 months, about 2 months to about 7 months, about 2 months to about 8 months, about 2 months to about 9 months, about 2 months to about 10 months, about 2 months to about 11 months, about 2 months to about 12 months, about 3 months to about 4 months, about 3 months to about 5 months, about 3 months to about 6 months, about 3 months to about 7 months, about 3 months to about 8 months, about 3 months to about 9 months, about 3 months to about 10 months, about 3 months to about 11 months, about 3 months to about 12 months, about 4 months to about 5 months, about 4 months to about 6 months, about 4 months to about 7 months, about 4 months to about 8 months, about 4 months to about 9 months, about 4 months to about 10 months, about 4 months to about 11 months, about 4 months to about 12 months, about 5 months to about 6 months, about 5 months to about 7 months, about 5 months to about 8 months, about 5 months to about 9 months, about 5 months to about 10 months, about 5 months to about 11 months, about 5 months to about 12 months, about 6 months to about 7 months, about 6 months to about 8 months, about 6 months to about 9 months, about 6 months to about 10 months, about 6 months to about 11 months, about 6 months to about 12 months, about 7 months to about 8 months, about 7 months to about 9 months, about 7 months to about 10 months, about 7 months to about 11 months, about 7 months to about 12 months, about 8 months to about 9 months, about 8 months to about 10 months, about 8 months to about 11 months, about 8 months to about 12 months, about 9 months to about 10 months, about 9 months to about 11 months, about 9 months to about 12 months, about 10 months to about 11 months, about 10 months to about 12 months, or about 11 months to about 12 months.

In other aspects of this embodiment, a first period of time during which a compound and/or a pharmaceutical composition disclosed herein may administered to an individual may be for, e.g., about 1 day to about 3 days, about 1 day to about 5 days, about 1 day to about 7 days, about 1 day to about 9 days, about 1 day to about 10 days, about 1 day to about 12 days, about 1 day to about 14 days, about 1 day to about 15 days, about 1 day to about 18 days, about 1 day to about 21 days, about 1 day to about 24 days, about 1 day to about 27 days, about 1 day to about 28 days, about 3 days to about 5 days, about 3 days to about 7 days, about 3 days to about 9 days, about 3 days to about 10 days, about 3 days to about 12 days, about 3 days to about 14 days, about 3 days to about 15 days, about 3 days to about 18 days, about 3 days to about 21 days, about 3 days to about 24 days, about 3 days to about 27 days, about 3 days to about 28 days, about 5 days to about 7 days, about 5 days to about 9 days, about 5 days to about 10 days, about 5 days to about 12 days, about 5 days to about 14 days, about 5 days to about 15 days, about 5 days to about 18 days, about 5 days to about 21 days, about 5 days to about 24 days, about 5 days to about 27 days, about 5 days to about 28 days, about 7 days to about 9 days, about 7 days to about 10 days, about 7 days to about 12 days, about 7 days to about 14 days, about 7 days to about 17 days, about 7 days to about 18 days, about 7 days to about 21 days, about 7 days to about 24 days, about 7 days to about 27 days, or about 7 days to about 28 days, about 10 days to about 12 days, about 10 days to about 14 days, about 10 days to about 17 days, about 10 days to about 18 days, about 10 days to about 21 days, about 10 days to about 24 days, about 10 days to about 27 days, about 10 days to about 28 days, and a second period of time during which a compound and/or a pharmaceutical composition disclosed herein may not be administered to an individual may be for, e.g., about 1 day, about 2 days, about 3 days, about 4 days, about 5 days, about 6 days, about 7 days, about 8 days, about 9 days, about 10 days, about 11 days, about 12 days, about 13 days, about 14 days, about 3 weeks, about 4 weeks, about 5 weeks, about 6 weeks, about 7 weeks, about 8 weeks, about 9 weeks, about 10 weeks, about 11 weeks, about 12 weeks, about 1 month, about 2 months, about 3 months, about 4 months, about 5 months, about 6 months, about 7 months, about 8 months, about 9 months, about 10 months, about 11 months, about 12 months, at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at most 1 day, at most 2 days, at most 3 days, at most 4 days, at most 5 days, at most 6 days, at most 7 days, at most 8 days, at most 9 days, at most 10 days, at most 11 days, at most 12 days, at most 13 days, at most 14 days, at most 3 weeks, at most 4 weeks, at most 5 weeks, at most 6 weeks, at most 7 weeks, at most 8 weeks, at most 9 weeks, at most 10 weeks, at most 11 weeks, at most 12 weeks, at most 1 month, at most 2 months, at most 3 months, at most 4 months, at most 5 months, at most 6 months, at most 7 months, at most 8 months, at most 9 months, at most 10 months, at most 11 months, or at most 12 months, about 1 day to about 3 days, about 1 day to about 5 days, about 1 day to about 7 days, about 1 day to about 9 days, about 1 day to about 10 days, about 1 day to about 12 days, about 1 day to about 14 days, about 1 day to about 15 days, about 1 day to about 18 days, about 1 day to about 21 days, about 1 day to about 24 days, about 1 day to about 27 days, about 1 day to about 28 days, about 3 days to about 5 days, about 3 days to about 7 days, about 3 days to about 9 days, about 3 days to about 10 days, about 3 days to about 12 days, about 3 days to about 14 days, about 3 days to about 15 days, about 3 days to about 18 days, about 3 days to about 21 days, about 3 days to about 24 days, about 3 days to about 27 days, about 3 days to about 28 days, about 5 days to about 7 days, about 5 days to about 9 days, about 5 days to about 10 days, about 5 days to about 12 days, about 5 days to about 14 days, about 5 days to about 15 days, about 5 days to about 18 days, about 5 days to about 21 days, about 5 days to about 24 days, about 5 days to about 27 days, about 5 days to about 28 days, about 7 days to about 9 days, about 7 days to about 10 days, about 7 days to about 12 days, about 7 days to about 14 days, about 7 days to about 17 days, about 7 days to about 18 days, about 7 days to about 21 days, about 7 days to about 24 days, about 7 days to about 27 days, about 7 days to about 28 days, about 10 days to about 12 days, about 10 days to about 14 days, about 10 days to about 17 days, about 10 days to about 18 days, about 10 days to about 21 days, about 10 days to about 24 days, about 10 days to about 27 days, about 10 days to about 28 days, about 1 week to about 2 weeks, about 1 week to about 3 weeks, about 1 week to about 4 weeks, about 1 week to about 5 weeks, about 1 week to about 6 weeks, about 1 week to about 7 weeks, about 1 week to about 8 weeks, about 1 week to about 9 weeks, about 1 week to about 10 weeks, about 1 week to about 11 weeks, about 1 week to about 12 weeks, about 2 weeks to about 3 weeks, about 2 weeks to about 4 weeks, about 2 weeks to about 5 weeks, about 2 weeks to about 6 weeks, about 2 weeks to about 7 weeks, about 2 weeks to about 8 weeks, about 2 weeks to about 9 weeks, about 2 weeks to about 10 weeks, about 2 weeks to about 11 weeks, about 2 weeks to about 12 weeks, about 3 weeks to about 4 weeks, about 3 weeks to about 5 weeks, about 3 weeks to about 6 weeks, about 3 weeks to about 7 weeks, about 3 weeks to about 8 weeks, about 3 weeks to about 9 weeks, about 3 weeks to about 10 weeks, about 3 weeks to about 11 weeks, about 3 weeks to about 12 weeks, about 4 weeks to about 5 weeks, about 4 weeks to about 6 weeks, about 4 weeks to about 7 weeks, about 4 weeks to about 8 weeks, about 4 weeks to about 9 weeks, about 4 weeks to about 10 weeks, about 4 weeks to about 11 weeks, about 4 weeks to about 12 weeks, about 5 weeks to about 6 weeks, about 5 weeks to about 7 weeks, about 5 weeks to about 8 weeks, about 5 weeks to about 9 weeks, about 5 weeks to about 10 weeks, about 5 weeks to about 11 weeks, about 5 weeks to about 12 weeks, about 6 weeks to about 7 weeks, about 6 weeks to about 8 weeks, about 6 weeks to about 9 weeks, about 6 weeks to about 10 weeks, about 6 weeks to about 11 weeks, about 6 weeks to about 12 weeks, about 7 weeks to about 8 weeks, about 7 weeks to about 9 weeks, about 7 weeks to about 10 weeks, about 7 weeks to about 11 weeks, about 7 weeks to about 12 weeks, about 8 weeks to about 9 weeks, about 8 weeks to about 10 weeks, about 8 weeks to about 11 weeks, about 8 weeks to about 12 weeks, about 9 weeks to about 10 weeks, about 9 weeks to about 11 weeks, about 9 weeks to about 12 weeks, about 10 weeks to about 11 weeks, about 10 weeks to about 12 weeks, about 11 weeks to about 12 weeks, about 1 month to about 2 months, about 1 month to about 3 months, about 1 month to about 4 months, about 1 month to about 5 months, about 1 month to about 6 months, about 1 month to about 7 months, about 1 month to about 8 months, about 1 month to about 9 months, about 1 month to about 10 months, about 1 month to about 11 months, about 1 month to about 12 months, about 2 months to about 3 months, about 2 months to about 4 months, about 2 months to about 5 months, about 2 months to about 6 months, about 2 months to about 7 months, about 2 months to about 8 months, about 2 months to about 9 months, about 2 months to about 10 months, about 2 months to about 11 months, about 2 months to about 12 months, about 3 months to about 4 months, about 3 months to about 5 months, about 3 months to about 6 months, about 3 months to about 7 months, about 3 months to about 8 months, about 3 months to about 9 months, about 3 months to about 10 months, about 3 months to about 11 months, about 3 months to about 12 months, about 4 months to about 5 months, about 4 months to about 6 months, about 4 months to about 7 months, about 4 months to about 8 months, about 4 months to about 9 months, about 4 months to about 10 months, about 4 months to about 11 months, about 4 months to about 12 months, about 5 months to about 6 months, about 5 months to about 7 months, about 5 months to about 8 months, about 5 months to about 9 months, about 5 months to about 10 months, about 5 months to about 11 months, about 5 months to about 12 months, about 6 months to about 7 months, about 6 months to about 8 months, about 6 months to about 9 months, about 6 months to about 10 months, about 6 months to about 11 months, about 6 months to about 12 months, about 7 months to about 8 months, about 7 months to about 9 months, about 7 months to about 10 months, about 7 months to about 11 months, about 7 months to about 12 months, about 8 months to about 9 months, about 8 months to about 10 months, about 8 months to about 11 months, about 8 months to about 12 months, about 9 months to about 10 months, about 9 months to about 11 months, about 9 months to about 12 months, about 10 months to about 11 months, about 10 months to about 12 months, or about 11 months to about 12 months.

In other aspects of this embodiment, a first period of time during which a compound and/or a pharmaceutical composition disclosed herein may administered to an individual may be for, e.g., about 1 week to about 2 weeks, about 1 week to about 3 weeks, about 1 week to about 4 weeks, about 1 week to about 5 weeks, about 1 week to about 6 weeks, about 1 week to about 7 weeks, about 1 week to about 8 weeks, about 1 week to about 9 weeks, about 1 week to about 10 weeks, about 1 week to about 11 weeks, about 1 week to about 12 weeks, about 2 weeks to about 3 weeks, about 2 weeks to about 4 weeks, about 2 weeks to about 5 weeks, about 2 weeks to about 6 weeks, about 2 weeks to about 7 weeks, about 2 weeks to about 8 weeks, about 2 weeks to about 9 weeks, about 2 weeks to about 10 weeks, about 2 weeks to about 11 weeks, about 2 weeks to about 12 weeks, about 3 weeks to about 4 weeks, about 3 weeks to about 5 weeks, about 3 weeks to about 6 weeks, about 3 weeks to about 7 weeks, about 3 weeks to about 8 weeks, about 3 weeks to about 9 weeks, about 3 weeks to about 10 weeks, about 3 weeks to about 11 weeks, about 3 weeks to about 12 weeks, about 4 weeks to about 5 weeks, about 4 weeks to about 6 weeks, about 4 weeks to about 7 weeks, about 4 weeks to about 8 weeks, about 4 weeks to about 9 weeks, about 4 weeks to about 10 weeks, about 4 weeks to about 11 weeks, about 4 weeks to about 12 weeks, about 5 weeks to about 6 weeks, about 5 weeks to about 7 weeks, about 5 weeks to about 8 weeks, about 5 weeks to about 9 weeks, about 5 weeks to about 10 weeks, about 5 weeks to about 11 weeks, about 5 weeks to about 12 weeks, about 6 weeks to about 7 weeks, about 6 weeks to about 8 weeks, about 6 weeks to about 9 weeks, about 6 weeks to about 10 weeks, about 6 weeks to about 11 weeks, about 6 weeks to about 12 weeks, about 7 weeks to about 8 weeks, about 7 weeks to about 9 weeks, about 7 weeks to about 10 weeks, about 7 weeks to about 11 weeks, about 7 weeks to about 12 weeks, about 8 weeks to about 9 weeks, about 8 weeks to about 10 weeks, about 8 weeks to about 11 weeks, about 8 weeks to about 12 weeks, about 9 weeks to about 10 weeks, about 9 weeks to about 11 weeks, about 9 weeks to about 12 weeks, about 10 weeks to about 11 weeks, about 10 weeks to about 12 weeks, or about 11 weeks to about 12 weeks, and a second period of time during which a compound and/or a pharmaceutical composition disclosed herein may not be administered to an individual may be for, e.g., about 1 day, about 2 days, about 3 days, about 4 days, about 5 days, about 6 days, about 7 days, about 8 days, about 9 days, about 10 days, about 11 days, about 12 days, about 13 days, about 14 days, about 3 weeks, about 4 weeks, about 5 weeks, about 6 weeks, about 7 weeks, about 8 weeks, about 9 weeks, about 10 weeks, about 11 weeks, about 12 weeks, about 1 month, about 2 months, about 3 months, about 4 months, about 5 months, about 6 months, about 7 months, about 8 months, about 9 months, about 10 months, about 11 months, about 12 months, at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at most 1 day, at most 2 days, at most 3 days, at most 4 days, at most 5 days, at most 6 days, at most 7 days, at most 8 days, at most 9 days, at most 10 days, at most 11 days, at most 12 days, at most 13 days, at most 14 days, at most 3 weeks, at most 4 weeks, at most 5 weeks, at most 6 weeks, at most 7 weeks, at most 8 weeks, at most 9 weeks, at most 10 weeks, at most 11 weeks, at most 12 weeks, at most 1 month, at most 2 months, at most 3 months, at most 4 months, at most 5 months, at most 6 months, at most 7 months, at most 8 months, at most 9 months, at most 10 months, at most 11 months, or at most 12 months, about 1 day to about 3 days, about 1 day to about 5 days, about 1 day to about 7 days, about 1 day to about 9 days, about 1 day to about 10 days, about 1 day to about 12 days, about 1 day to about 14 days, about 1 day to about 15 days, about 1 day to about 18 days, about 1 day to about 21 days, about 1 day to about 24 days, about 1 day to about 27 days, about 1 day to about 28 days, about 3 days to about 5 days, about 3 days to about 7 days, about 3 days to about 9 days, about 3 days to about 10 days, about 3 days to about 12 days, about 3 days to about 14 days, about 3 days to about 15 days, about 3 days to about 18 days, about 3 days to about 21 days, about 3 days to about 24 days, about 3 days to about 27 days, about 3 days to about 28 days, about 5 days to about 7 days, about 5 days to about 9 days, about 5 days to about 10 days, about 5 days to about 12 days, about 5 days to about 14 days, about 5 days to about 15 days, about 5 days to about 18 days, about 5 days to about 21 days, about 5 days to about 24 days, about 5 days to about 27 days, about 5 days to about 28 days, about 7 days to about 9 days, about 7 days to about 10 days, about 7 days to about 12 days, about 7 days to about 14 days, about 7 days to about 17 days, about 7 days to about 18 days, about 7 days to about 21 days, about 7 days to about 24 days, about 7 days to about 27 days, about 7 days to about 28 days, about 10 days to about 12 days, about 10 days to about 14 days, about 10 days to about 17 days, about 10 days to about 18 days, about 10 days to about 21 days, about 10 days to about 24 days, about 10 days to about 27 days, about 10 days to about 28 days, about 1 week to about 2 weeks, about 1 week to about 3 weeks, about 1 week to about 4 weeks, about 1 week to about 5 weeks, about 1 week to about 6 weeks, about 1 week to about 7 weeks, about 1 week to about 8 weeks, about 1 week to about 9 weeks, about 1 week to about 10 weeks, about 1 week to about 11 weeks, about 1 week to about 12 weeks, about 2 weeks to about 3 weeks, about 2 weeks to about 4 weeks, about 2 weeks to about 5 weeks, about 2 weeks to about 6 weeks, about 2 weeks to about 7 weeks, about 2 weeks to about 8 weeks, about 2 weeks to about 9 weeks, about 2 weeks to about 10 weeks, about 2 weeks to about 11 weeks, about 2 weeks to about 12 weeks, about 3 weeks to about 4 weeks, about 3 weeks to about 5 weeks, about 3 weeks to about 6 weeks, about 3 weeks to about 7 weeks, about 3 weeks to about 8 weeks, about 3 weeks to about 9 weeks, about 3 weeks to about 10 weeks, about 3 weeks to about 11 weeks, about 3 weeks to about 12 weeks, about 4 weeks to about 5 weeks, about 4 weeks to about 6 weeks, about 4 weeks to about 7 weeks, about 4 weeks to about 8 weeks, about 4 weeks to about 9 weeks, about 4 weeks to about 10 weeks, about 4 weeks to about 11 weeks, about 4 weeks to about 12 weeks, about 5 weeks to about 6 weeks, about 5 weeks to about 7 weeks, about 5 weeks to about 8 weeks, about 5 weeks to about 9 weeks, about 5 weeks to about 10 weeks, about 5 weeks to about 11 weeks, about 5 weeks to about 12 weeks, about 6 weeks to about 7 weeks, about 6 weeks to about 8 weeks, about 6 weeks to about 9 weeks, about 6 weeks to about 10 weeks, about 6 weeks to about 11 weeks, about 6 weeks to about 12 weeks, about 7 weeks to about 8 weeks, about 7 weeks to about 9 weeks, about 7 weeks to about 10 weeks, about 7 weeks to about 11 weeks, about 7 weeks to about 12 weeks, about 8 weeks to about 9 weeks, about 8 weeks to about 10 weeks, about 8 weeks to about 11 weeks, about 8 weeks to about 12 weeks, about 9 weeks to about 10 weeks, about 9 weeks to about 11 weeks, about 9 weeks to about 12 weeks, about 10 weeks to about 11 weeks, about 10 weeks to about 12 weeks, about 11 weeks to about 12 weeks, about 1 month to about 2 months, about 1 month to about 3 months, about 1 month to about 4 months, about 1 month to about 5 months, about 1 month to about 6 months, about 1 month to about 7 months, about 1 month to about 8 months, about 1 month to about 9 months, about 1 month to about 10 months, about 1 month to about 11 months, about 1 month to about 12 months, about 2 months to about 3 months, about 2 months to about 4 months, about 2 months to about 5 months, about 2 months to about 6 months, about 2 months to about 7 months, about 2 months to about 8 months, about 2 months to about 9 months, about 2 months to about 10 months, about 2 months to about 11 months, about 2 months to about 12 months, about 3 months to about 4 months, about 3 months to about 5 months, about 3 months to about 6 months, about 3 months to about 7 months, about 3 months to about 8 months, about 3 months to about 9 months, about 3 months to about 10 months, about 3 months to about 11 months, about 3 months to about 12 months, about 4 months to about 5 months, about 4 months to about 6 months, about 4 months to about 7 months, about 4 months to about 8 months, about 4 months to about 9 months, about 4 months to about 10 months, about 4 months to about 11 months, about 4 months to about 12 months, about 5 months to about 6 months, about 5 months to about 7 months, about 5 months to about 8 months, about 5 months to about 9 months, about 5 months to about 10 months, about 5 months to about 11 months, about 5 months to about 12 months, about 6 months to about 7 months, about 6 months to about 8 months, about 6 months to about 9 months, about 6 months to about 10 months, about 6 months to about 11 months, about 6 months to about 12 months, about 7 months to about 8 months, about 7 months to about 9 months, about 7 months to about 10 months, about 7 months to about 11 months, about 7 months to about 12 months, about 8 months to about 9 months, about 8 months to about 10 months, about 8 months to about 11 months, about 8 months to about 12 months, about 9 months to about 10 months, about 9 months to about 11 months, about 9 months to about 12 months, about 10 months to about 11 months, about 10 months to about 12 months, or about 11 months to about 12 months.

Various routes of administration can be useful for administering a therapeutic compound disclosed herein, according to a method of treating a gender-biased immune disorder disclosed herein. A pharmaceutical composition may be administered to an individual by any of a variety of means depending, e.g., on the type of immune disorder to be treated, the location of immune disorder to be treated, the specific therapeutic compound or composition used, or other compound to be included in the composition, and the history, risk factors and symptoms of the individual. As such, topical, enteral or parenteral routes of administration may be suitable for of treating a gender-biased immune disorder disclosed herein and such routes include both local and systemic delivery of a therapeutic compound or composition disclosed herein. Compositions comprising either a single therapeutic compound disclosed herein, or two or more therapeutic compounds disclosed herein are intended for inhaled, topical, intranasal, sublingual, intravenous, rectal and/or vaginal use may be prepared according to any method known to the art for the manufacture of pharmaceutical compositions.

A pharmaceutical composition disclosed herein can be administered to an individual in a single formulation or in separate formulations, for combined, simultaneous or sequential administration. In one embodiment, an individual is administered a first composition comprising a progesterone agonist and a second composition comprising another therapeutic compound capable of modulating activity of a hormone. In aspects of this embodiment, an individual is administered a first composition comprising a progesterone and a second composition comprising at least one other therapeutic compound capable of modulating activity of a hormone. In other aspects of this embodiment, the at least one other therapeutic compound capable of modulating activity of a hormone includes, without limitation, a SPRM, an estrogen antagonist, a SERM, an estrogen biosynthesis enzyme inhibitor, an androgen agonist, a SARM, or any combination thereof. In yet other aspects of this embodiment, an individual is administered a first composition comprising a progesterone and a second composition comprising an estrogen biosynthesis enzyme inhibitor. In still other aspects of this embodiment, an individual is administered a first composition comprising a progesterone and a second composition comprising an aromatase inhibitor.

In another embodiment, an individual is administered a composition comprising a progesterone agonist and another therapeutic compound capable of modulating activity of a hormone. In aspects of this embodiment, an individual is administered a composition comprising a progesterone and at least one other therapeutic compound capable of modulating activity of a hormone. In other aspects of this embodiment, the at least one other therapeutic compound capable of modulating activity of a hormone includes, without limitation, a SPRM, an estrogen antagonist, a SERM, an estrogen biosynthesis enzyme inhibitor, an androgen agonist, a SARM, or any combination thereof. In yet other aspects of this embodiment, an individual is administered a composition comprising a progesterone and an estrogen biosynthesis enzyme inhibitor. In still other aspects of this embodiment, an individual is administered a composition comprising a progesterone and an aromatase inhibitor.

In another embodiment, an individual is administered a first composition comprising a progesterone agonist and a second composition comprising another therapeutic compound capable of modulating activity of a hormone for a plurality of cycles, wherein each of the plurality of cycles comprises administering the first and second compositions for a first period of time followed by a second period of time where the first and second compositions are not administered to the individual. In aspects of this embodiment, an individual is administered a first composition comprising a progesterone and a second composition comprising another therapeutic compound capable of modulating activity of a hormone for a plurality of cycles, wherein each of the plurality of cycles comprises administering the first and second compositions for a first period of time followed by a second period of time where the first and second compositions are not administered to the individual. In other aspects of this embodiment, the at least one other therapeutic compound capable of modulating activity of a hormone includes, without limitation, a SPRM, an estrogen antagonist, a SERM, an estrogen biosynthesis enzyme inhibitor, an androgen agonist, a SARM, or any combination thereof. In yet other aspects of this embodiment, an individual is administered a first composition comprising a progesterone and a second composition comprising an estrogen biosynthesis enzyme inhibitor for a plurality of cycles, wherein each of the plurality of cycles comprises administering the first and second compositions for a first period of time followed by a second period of time where the first and second compositions are not administered to the individual. In still other aspects of this embodiment, an individual is administered a first composition comprising a progesterone and a second composition comprising an aromatase inhibitor for a plurality of cycles, wherein each of the plurality of cycles comprises administering the first and second compositions for a first period of time followed by a second period of time where the first and second compositions are not administered to the individual. In an aspect of this embodiment, the first and second compositions are administered to an individual for a plurality of cycles that is maintained for a definite period of time. In another aspect of this embodiment, the first and second compositions are administered to an individual for a plurality of cycles that is perpetually maintained.

In aspects of this embodiment, an individual is administered a first composition comprising progesterone and a second composition comprising at least one other therapeutic compound capable of modulating activity of a hormone for a plurality of cycles, wherein each of the plurality of cycles comprises administering the first and second compositions for a first period of time of about 10 days to about 28 days followed by a second period of time of about 10 days to about 28 days where the first and second compositions are not administered to the individual. In other aspects of this embodiment, an individual is administered a first composition comprising progesterone and a second composition comprising at least one other therapeutic compound capable of modulating activity of a hormone for a plurality of cycles, wherein each of the plurality of cycles comprises administering the first and second compositions for a first period of time of about 10 days to about 21 days followed by a second period of time of about 10 days to about 21 days where the first and second compositions are not administered to the individual. In yet other aspects of this embodiment, an individual is administered a first composition comprising progesterone and a second composition comprising at least one other therapeutic compound capable of modulating activity of a hormone for a plurality of cycles, wherein each of the plurality of cycles comprises administering the first and second compositions for a first period of time of about 10 days to about 14 days followed by a second period of time of about 10 days to about 14 days where the first and second compositions are not administered to the individual. In still other aspects of this embodiment, an individual is administered a first composition comprising progesterone and a second composition comprising at least one other therapeutic compound capable of modulating activity of a hormone for a plurality of cycles, wherein each of the plurality of cycles comprises administering the first and second compositions for a first period of time of about 14 days followed by a second period of time of about 14 days where the first and second compositions are not administered to the individual. In an aspect of this embodiment, the first and second compositions are administered to an individual for a plurality of cycles that is maintained for a definite period of time. In another aspect of this embodiment, the first and second compositions are administered to an individual for a plurality of cycles that is perpetually maintained.

In aspects of this embodiment, an individual is administered a first composition comprising progesterone and a second composition comprising at least one of a SPRM, an estrogen antagonist, a SERM, an estrogen biosynthesis enzyme inhibitor, an androgen agonist, a SARM, or any combination thereof for a plurality of cycles, wherein each of the plurality of cycles comprises administering the first and second compositions for a first period of time of about 10 days to about 28 days followed by a second period of time of about 10 days to about 28 days where the first and second compositions are not administered to the individual. In other aspects of this embodiment, an individual is administered a first composition comprising progesterone and a second composition comprising at least one of a SPRM, an estrogen antagonist, a SERM, an estrogen biosynthesis enzyme inhibitor, an androgen agonist, a SARM, or any combination thereof for a plurality of cycles, wherein each of the plurality of cycles comprises administering the first and second compositions for a first period of time of about 10 days to about 21 days followed by a second period of time of about 10 days to about 21 days where the first and second compositions are not administered to the individual. In yet other aspects of this embodiment, an individual is administered a first composition comprising progesterone and a second composition comprising at least one of a SPRM, an estrogen antagonist, a SERM, an estrogen biosynthesis enzyme inhibitor, an androgen agonist, a SARM, or any combination thereof for a plurality of cycles, wherein each of the plurality of cycles comprises administering the first and second compositions for a first period of time of about 10 days to about 14 days followed by a second period of time of about 10 days to about 14 days where the first and second compositions are not administered to the individual. In still other aspects of this embodiment, an individual is administered a first composition comprising progesterone and a second composition comprising at least one of a SPRM, an estrogen antagonist, a SERM, an estrogen biosynthesis enzyme inhibitor, an androgen agonist, a SARM, or any combination thereof for a plurality of cycles, wherein each of the plurality of cycles comprises administering the first and second compositions for a first period of time of about 14 days followed by a second period of time of about 14 days where the first and second compositions are not administered to the individual. In an aspect of this embodiment, the first and second compositions are administered to an individual for a plurality of cycles that is maintained for a definite period of time. In another aspect of this embodiment, the first and second compositions are administered to an individual for a plurality of cycles that is perpetually maintained.

In aspects of this embodiment, an individual is administered a first composition comprising a progesterone and a second composition comprising an estrogen biosynthesis enzyme inhibitor for a plurality of cycles, wherein each of the plurality of cycles comprises administering the first and second compositions for a first period of time of about 10 days to about 28 days followed by a second period of time of about 10 days to about 28 days where the first and second compositions are not administered to the individual. In other aspects of this embodiment, an individual is administered a first composition comprising a progesterone and a second composition comprising an estrogen biosynthesis enzyme inhibitor for a plurality of cycles, wherein each of the plurality of cycles comprises administering the first and second compositions for a first period of time of about 10 days to about 21 days followed by a second period of time of about 10 days to about 21 days where the first and second compositions are not administered to the individual. In yet other aspects of this embodiment, an individual is administered a first composition comprising a progesterone and a second composition comprising an estrogen biosynthesis enzyme inhibitor for a plurality of cycles, wherein each of the plurality of cycles comprises administering the first and second compositions for a first period of time of about 10 days to about 14 days followed by a second period of time of about 10 days to about 14 days where the first and second compositions are not administered to the individual. In still other aspects of this embodiment, an individual is administered a first composition comprising a progesterone and a second composition comprising an estrogen biosynthesis enzyme inhibitor for a plurality of cycles, wherein each of the plurality of cycles comprises administering the first and second compositions for a first period of time of about 14 days followed by a second period of time of about 14 days where the first and second compositions are not administered to the individual. In an aspect of this embodiment, the first and second compositions are administered to an individual for a plurality of cycles that is maintained for a definite period of time. In another aspect of this embodiment, the first and second compositions are administered to an individual for a plurality of cycles that is perpetually maintained.

In aspects of this embodiment, an individual is administered a first composition comprising a progesterone and a second composition comprising an aromatase inhibitor for a plurality of cycles, wherein each of the plurality of cycles comprises administering the first and second compositions for a first period of time pf about 10 days to about 28 days followed by a second period of time of about 10 days to about 28 days where the first and second compositions are not administered to the individual. In other aspects of this embodiment, an individual is administered a first composition comprising a progesterone and a second composition comprising an aromatase inhibitor for a plurality of cycles, wherein each of the plurality of cycles comprises administering the first and second compositions for a first period of time pf about 10 days to about 21 days followed by a second period of time of about 10 days to about 21 days where the first and second compositions are not administered to the individual. In yet other aspects of this embodiment, an individual is administered a first composition comprising a progesterone and a second composition comprising an aromatase inhibitor for a plurality of cycles, wherein each of the plurality of cycles comprises administering the first and second compositions for a first period of time pf about 10 days to about 14 days followed by a second period of time of about 10 days to about 14 days where the first and second compositions are not administered to the individual. In still other aspects of this embodiment, an individual is administered a first composition comprising a progesterone and a second composition comprising an aromatase inhibitor for a plurality of cycles, wherein each of the plurality of cycles comprises administering the first and second compositions for a first period of time pf about 14 days followed by a second period of time of about 14 days where the first and second compositions are not administered to the individual. In an aspect of this embodiment, the first and second compositions are administered to an individual for a plurality of cycles that is maintained for a definite period of time. In another aspect of this embodiment, the first and second compositions are administered to an individual for a plurality of cycles that is perpetually maintained.

In aspects of this embodiment, an individual is administered a first composition comprising a progesterone and a second composition comprising a Letrozole for a plurality of cycles, wherein each of the plurality of cycles comprises administering the first and second compositions for a first period of time pf about 10 days to about 28 days followed by a second period of time of about 10 days to about 28 days where the first and second compositions are not administered to the individual. In other aspects of this embodiment, an individual is administered a first composition comprising a progesterone and a second composition comprising a Letrozole for a plurality of cycles, wherein each of the plurality of cycles comprises administering the first and second compositions for a first period of time pf about 10 days to about 21 days followed by a second period of time of about 10 days to about 21 days where the first and second compositions are not administered to the individual. In yet other aspects of this embodiment, an individual is administered a first composition comprising a progesterone and a second composition comprising a Letrozole for a plurality of cycles, wherein each of the plurality of cycles comprises administering the first and second compositions for a first period of time pf about 10 days to about 14 days followed by a second period of time of about 10 days to about 14 days where the first and second compositions are not administered to the individual. In still other aspects of this embodiment, an individual is administered a first composition comprising a progesterone and a second composition comprising a Letrozole for a plurality of cycles, wherein each of the plurality of cycles comprises administering the first and second compositions for a first period of time pf about 14 days followed by a second period of time of about 14 days where the first and second compositions are not administered to the individual. In an aspect of this embodiment, the first and second compositions are administered to an individual for a plurality of cycles that is maintained for a definite period of time. In another aspect of this embodiment, the first and second compositions are administered to an individual for a plurality of cycles that is perpetually maintained.

A pharmaceutical composition disclosed herein can also be administered to an individual in combination with other therapeutic compounds to increase the overall therapeutic effect of the treatment. The use of multiple compounds to treat an indication can increase the beneficial effects while reducing the presence of side effects.

Aspects of the present specification may also be described as follows:
1. A composition comprising one or more therapeutic compound capable of modulating activity of a hormone.
2. The composition according to embodiment 1, wherein the therapeutic compound reduces a level of a hormone.
3. The composition according to embodiment 1 or embodiment 2, wherein the therapeutic compound reduces an activity of a hormone.
4. The composition according to any one of embodiments 1-3, wherein the therapeutic compound reduces a level of an estrogen.
5. The composition according to embodiment 4, wherein the therapeutic compound reduces a level of an estrogen by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90% or at most 95%, or about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.
6. The composition according to any one of embodiments 1-5, wherein the therapeutic compound reduces a level of a biosynthetic enzyme for an estrogen.
7. The composition according to embodiment 6, wherein the therapeutic compound reduces a level of a biosynthetic enzyme for an estrogen by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90% or at most 95%, or about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.
8. The composition according to any one of embodiments 1-7, wherein the therapeutic compound reduces an activity of a biosynthetic enzyme for an estrogen.
9. The composition according to embodiment 8, wherein the therapeutic compound reduces an activity of a biosynthetic enzyme for an estrogen by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90% or at most 95%, or about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.
10. The composition according to any one of embodiments 6-9, wherein the estrogen is an estrone (E1), an estradiol (E2), an estriol (E3), estetrol (E4), or any combination thereof.
11. The composition according to any one of embodiments 1-10, wherein the therapeutic compound reduces a level of an androgen.
12. The composition according to embodiment 11, wherein the therapeutic compound reduces a level of an androgen by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90% or at most 95%, or about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.
13. The composition according to any one of embodiments 1-12, wherein the therapeutic compound reduces a level of a biosynthetic enzyme for an androgen.
14. The composition according to embodiment 13, wherein the therapeutic compound reduces a level of a biosynthetic enzyme for an androgen by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90% or at most 95%, or about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.
15. The composition according to any one of embodiments 1-14, wherein the therapeutic compound reduces an activity of a biosynthetic enzyme for an androgen.
16. The composition according to embodiment 15, wherein the therapeutic compound reduces an activity of a biosynthetic enzyme for an androgen by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90% or at most 95%, or about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.
17. The composition according to any one of embodiments 11-16, wherein the androgen is an Androstenediol, an Androstenedione, an Androsterone, a Dehydroepiandrosterone (DHEA), a Dihydrotestosterone (DHT), a Testosterone, or any combination thereof.
18. The composition according to any one of embodiments 1-17, wherein the therapeutic compound reduces a level of a progesterone.
19. The composition according to embodiment 18, wherein the therapeutic compound reduces a level of a progesterone by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90% or at most 95%, or about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.
20. The composition according to any one of embodiments 1-19, wherein the therapeutic compound reduces a level of a biosynthetic enzyme for a progesterone.
21. The composition according to embodiment 20, wherein the therapeutic compound reduces a level of a biosynthetic enzyme for a progesterone by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90% or at most 95%, or about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.
22. The composition according to any one of embodiments 1-21, wherein the therapeutic compound reduces an activity of a biosynthetic enzyme for a progesterone.
23. The composition according to embodiment 22, wherein the therapeutic compound reduces an activity of a biosynthetic enzyme for a progesterone by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90% or at most 95%, or about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.
24. The composition according to any one of embodiments 18-23, wherein the progesterone is a 17-hydroxyprogesterone, a 17-hydroxypregnenolone, a pregnenolone, a progesterone, or any combination thereof.
25. The composition according to any one of embodiments 1-24, wherein the therapeutic compound reduces a level of a gender-biased immune disorder-causing antigen.
26. The composition according to embodiment 25, wherein the therapeutic compound reduces a level of an immune-disorder causing antigen by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90% or at most 95%, or about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.
27. The composition according to any one of embodiments 1-26, wherein the therapeutic compound reduces antigenicity of a gender-biased immune disorder-causing antigen.
28. The composition according to embodiment 27, wherein the therapeutic compound reduces antigenicity of a gender-biased immune disorder-causing antigen by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90% or at most 95%, or about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.
29. The composition according to any one of embodiments 1-28, wherein the therapeutic compound reduces the ability of an immune system to react to a gender-biased immune disorder-causing antigen.
30. The composition according to embodiment 29, wherein the therapeutic compound reduces the ability of an immune system to react to a gender-biased immune disorder-causing antigen by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90% or at most 95%, or about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.
31. The composition according to any one of embodiments 25-30, wherein the gender-biased immune disorder-causing antigen is a cytotoxic T lymphocyte (CTL) antigen, CD8⁺ T cell antigen and/or CD4⁺ T cell antigen.
32. The composition according to any one of embodiments 25-30, wherein the gender-biased immune disorder-causing antigen is a MHC II antigen.
33. The composition according to any one of embodiments 1-32, wherein the therapeutic compound reduces antigenicity of a SLE antigen and/or an α-SLE antibody.
34. The composition according to embodiment 33, wherein the therapeutic compound reduces antigenicity of a SLE antigen and/or an α-SLE antibody by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90% or at most 95%, or about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.
35. The composition according to any one of embodiments 1-32, wherein the therapeutic compound reduces antigenicity of a P1/P2 antigen and/or an α-P1/P2 ribosomal protein antibody.
36. The composition according to embodiment 35, wherein the therapeutic compound reduces antigenicity of a P1/P2 antigen and/or an α-P1/P2 ribosomal protein antibody by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90% or at most 95%, or about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.
37. The composition according to any one of embodiments 1-32, wherein the therapeutic compound reduces antigenicity of a ssDNA antigen and/or an α-ssDNA antibody.
38. The composition according to embodiment 37, wherein the therapeutic compound reduces antigenicity of a ssDNA antigen and/or an α-ssDNA antibody by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90% or at most 95%, or about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.
39. The composition according to any one of embodiments 1-38, wherein the therapeutic compound reduces a level of a Th1-type cytokine.
40. The composition according to embodiment 39, wherein the therapeutic compound reduces a level of a Th1-type cytokine by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90% or at most 95%, or about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.
41. The composition according to embodiment 39 or embodiment 40, wherein the Th1-type cytokine is an Interferon gamma (IFγ), a Tumor necrosis factor alpha (TNFα), an Interleukin-2 (IL-2), an Interleukin-6 (IL-6), an Interleukin-12 (IL-12), an Interleukin-23 (IL-23), or any combination thereof.
42. The composition according to any one of embodiments 1-38, wherein the therapeutic compound reduces a level of a Th2-type cytokine.
43. The composition according to embodiments 42, wherein the therapeutic compound reduces a level of a Th2-type cytokine by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90% or at most 95%, or about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.
44. The composition according to embodiment 42 or embodiment 43, wherein the Th1-type cytokine is an Interleukin-4 (IL-4), an Interleukin-5 (IL-5), an Interleukin-10 (IL-10), an Interleukin-13 (IL-13), or any combination thereof.
45. The composition according to any one of embodiments 1-44, wherein the therapeutic compound reduces the level of an inflammation inducing molecule.
46. The composition according to embodiment 45, wherein the inflammation inducing molecule comprises substance P (SP), calcitonin gene-related peptide (CGRP), glutamate, or a combination thereof.
47. The composition according to embodiment 46, wherein the therapeutic compound reduces the level of SP, CGRP, glutamate, or a combination thereof by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90% or at most 95%, or about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.
48. The composition according to any one of embodiments 1-47, wherein the therapeutic compound reduces the level of an inflammation inducing prostaglandin.
49. The composition according to embodiment 49, wherein the level of the inflammation inducing prostaglandin is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90% or at most 95%, or about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.
50. The composition according to any one of embodiments 1-49, wherein the therapeutic compound stimulates a PPAR signaling pathway.
51. The composition according to embodiment 50, wherein the PPAR signaling pathway is stimulated by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90% or at most 95%, or about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.
52. The composition according to any one of embodiments 1-51, wherein the therapeutic compound reduces apoptosis.
53. The composition according embodiment 52, wherein the therapeutic compound reduces apoptosis by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90% or at most 95%, or about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.
54. The composition according to any one of embodiments 1-51, wherein the therapeutic compound induces apoptosis of Macrophage M1 cells, promotes differentiation of Macrophage M2 cells, or both.
55. The composition according to any one of embodiments 1-54, wherein the therapeutic compound includes a progesterone agonist, a SPRM, a progesterone antagonist, an estrogen antagonist, a SERM, an estrogen biosynthesis enzyme inhibitor, an estrogen agonist, an androgen agonist, a SARM, an androgen antagonist, or any combination thereof.
56. The composition according to embodiment 55, wherein the progesterone agonist is a full progesterone agonist or a partial progesterone agonist.
57. The composition according to embodiment 55, wherein the progesterone agonist includes 11-Dehydroprogesterone, 11-Deoxycorticosterone, 17-Hydroxyprogesterone, 19-Norprogesterone, Algestone, Algestone acetonide, Algestone acetophenide, Allylestrenol, Altrenogest, Amadinone, Amadinone acetate, Anagestone, Anagestone acetate, Chlormadinone, Chlormadinone acetate, Cingestol, Cismadinone, Cismadinone acetate, Clogestone, Clogestone acetate, Clomegestone, Cyproterone, Cyproterone acetate, Delmadinone, Delmadinone acetate, Demegestone, Deoxycorticosterone, Desogestrel, Dienogest, Dimethisterone, Drospirenone, Dydrogesterone, Edogesterone, Ethisterone, Ethynerone, Ethynodiol, Ethynodiol diacetate, Etonogestrel, Etynodiol, Flugestone, Flugestone acetate, Gestaclone, Gestadienol, Gestodene, Gestonorone, Gestonorone caproate, Gestovister, Gestrinone, Gestronol, Haloprogesterone, Hydromadinone, Hydroxyprogesterone acetate, Hydroxyprogesterone caproate, Hydroxyprogesterone heptanoate, Hydroxyprogesterone hexanoate, Levonorgestrel, Lutenyl, Lynestrenol, Medrogestone, Medroxyprogesterone, Medroxyprogesterone acetate, Megestrol, Megestrol acetate, Melengestrol, Melengestrol acetate, Methylestrenolone, Methynodiol, Methynodiol diacetate, Metogest, Nandrolone, Nestorone, Nomegestrol, Nomegestrol acetate, Norelgestromin, Norethindrone, Norethindrone acetate, Norethisterone, Norethisterone acetate, Norethisterone acetate oxime, Norethisterone enanthate, Norethynodrel, Norethynodrel diacetate, Norgesterone, Norgestimate, Norgestomet, Norgestrel, Norgestrienone, Norvinisterone, Org-2058, Oxogestone, Oxogestone phenpropionate, Pentagestrone, Pregnane, Progesterone, Proligestone, Promegestone, Quingestanol, Quingestanol acetate, Quingestrone, Segesterone, Spironenone, Spironolactone, Tanaproget, Tibolone, Tigestol, Tosagestin, Trengestone, Trimegestone, ZM-182,345, or any combination thereof.
58. The composition according to embodiment 55, wherein the SPRM includes Asoprisnil, Asoprisnil ecamate, J1042, LG-120,838, Telapristone, or any combination thereof.
59. The composition according to embodiment 55, wherein the progesterone antagonist is a full progesterone antagonist or a partial progesterone antagonist.
60. The composition according to embodiment 55, wherein the progesterone antagonist includes Aglepristone, Lilopristone, Lonaprisan, Mifepristone, Onapristone, Toripristone, ZM-150,271, or ZM-172,406.
61. The composition according to embodiment 55, wherein the estrogen antagonist is a full estrogen antagonist, a partial estrogen antagonist, or an inverse estrogen agonist.
62. The composition according to embodiment 55, wherein the estrogen antagonist includes 2-Hydroxyestrone, 16-Hydroxyestrone, Acefluranol, Clometerone, Delmadinone, Dimepregnen, Epitiostanol, Fulvestrant, Gestrinone, ICI-164,384, Mepitiostane, MPP, PHTPP, RU-58,668, SS1020, ZK-164,015, ZK-191,703, or any combination thereof.
63. The composition according to embodiment 55, wherein the SERM includes 2-Hydroxyestrone, Acolbifene, Afimoxifene, Arzoxifene, Bazedoxifene, Clomifene, Clomifenoxide, Cyclofenil, Droloxifene, Enclomifene, Endoxifen, Epimestrol, Femarelle, Fispemifene, GW5638, Idoxifene, Lasofoxifene, Levormeloxifene, Menerba, Mepitiostane, Miproxifene, Nafoxidine, Nitromifene, Ormeloxifene, Ospemifene, Panomifene, Pipendoxifene, Prinaberel, Raloxifene, Resveratrol, SS1010, Sivifene, Tamoxifen, TAS-108, Tesmilifene, Tibolone, Toremifene, Trioxifene, Y-134, Zindoxifene, Zuclomifene, or any combination thereof.
64. The composition according to embodiment 55, wherein the estrogen biosynthesis enzyme inhibitor is an aromatase inhibitor, a CYP17A1 inhibitor, a 3-β-HSD inhibitor, a 17β-HSD inhibitor, or any combination thereof.
65. The composition according to embodiment 64, wherein the aromatase inhibitor includes 1,4,6-Androstatriene-3,17-dione (ATD), 4-Androstene-3,6,17-trione (6-OXO), 4-Cyclohexylaniline, 4-Hydroxyand rostenedione, 4-Hydroxytestosterone, 5α-DHNET, Abyssinone II, Aminoglutethimide, Anastrozole, Ascorbic acid (Vitamin C), Atamestane, Bifonazole, CGP-45,688, CGS-47,645, Clotrimazole, DHT, Difeconazole, Econazole, Exemestane, Fadrozole, Fenarimol, Finrozole, Formestane, Imazalil, Isoconazole, Ketoconazole, Letrozole, Liarozole, MEN-11066, Miconazole, Minamestane, Nimorazole, NKS01, Norethindrone, ORG-33,201, Penconazole, Plomestane, Prochloraz, Propioconazole, Pyridoglutethimide, Rogletimide, Rotenone, Talarozole, Testolactone, Tioconazole, Triadimefon, Triadimenol, Troglitazone, Vorozole, YM511, Zinc, or any combination thereof.
66. The composition according to embodiment 64, wherein the CYP17A1 inhibitor includes a 17α-Hydroxylase inhibitor, a 17,20-Lyase inhibitor, and a 20,22-Desmolase inhibitor, or any combination thereof.
67. The composition according to embodiment 66, wherein the 17α-Hydroxylase inhibitor includes 22-ABC, 22-Oxime, Abiraterone, Bifonazole, Clotrimazole, Cyanoketone, Cyproterone, Danazol, Desogestrel, Econazole, Galeterone, Gestrinone, Isoconazole, Ketoconazole, L-39, Levonorgestrel, Liarozole, or any combination thereof.
68. The composition according to embodiment 66, wherein the 17,20-Lyase inhibitor includes 22-ABC, 22-Oxime, Abiraterone, Bifonazole, Clotrimazole, Cyanoketone, Cyproterone, Danazol, Desogestrel, Econazole, Galeterone, Gestrinone, Isoconazole, Ketoconazole, L-39, Levonorgestrel, Liarozole, LY-207,320, MDL-27,302, Miconazole, Mifepristone, Norethisterone, Orteronel, Pioglitazone, Rosiglitazone, Spironolactone, Stanozolol, SU-10,603, Tioconazole, TGF-β, Troglitazone, VN/87-1, YM116, or any combination thereof.
69. The composition according to embodiment 66, wherein the 20,22-Desmolase inhibitor includes 22-ABC, 3,3′-Dimethoxybenzidine, 3-Methoxybenzidine, Aminoglutethimide, Cyanoketone, Danazol, Etomidate, Mitotane, Trilostane, or any combination thereof.
70. The composition according to embodiment 64, wherein the 3-β-HSD inhibitor includes 4-MA, Azastene, Cyanoketone, Danazol, Desogestrel, Epostane, Genistein, Gestrinone, Levonorgestrel, Medroxyprogesterone, Med roxyprogesterone acetate, Metyrapone, Norethisterone, Oxymetholone, Pioglitazone, Rosiglitazone, Trilostane, Troglitazone, or any combination thereof.
71. The composition according to embodiment 64, wherein the 17β-HSD inhibitor includes Danazol, Simvastatin, or any combination thereof.
72. The composition according to embodiment 55, wherein the estrogen agonist is a full estrogen agonist or a partial estrogen agonist.
73. The composition according to embodiment 55, wherein the estrogen agonist includes 5α-Androstane-3β,17β-diol, 16α-Hydroxyestrone, Alestramustine, Almestrone, Benzestrol, Bifluranol, Broparestrol, Carbestrol, Chlorotrianisene, Cloxestradiol, Daidzein, DHEA, Dienestrol, Diethylstilbestrol, Diethylstilbestrol diphosphate, Diosgenin, Doisynoestrol, DPN, Epiestriol, Epimestrol, Eptamestrol/Etamestrol, ERB-041, Equilenin, Equilin, Estetrol, Estradiol, Estradiol benzoate, Estradiol butyrylacetate, Estradiol cypionate, Estradiol dienanthate, Estradiol dipropionate, Estradiol diundecylate, Estradiol diundecylenate, Estradiol enanthate, Estradiol furoate, Estradiol hemihydrate, Estradiol hemisuccinate, Estradiol hexahydrobenzoate, Estradiol monopropionate, Estradiol palmitate, Estradiol pivalate, Estradiol propoxyphenylpropionate, Estradiol stearate, Estradiol succinate, Estradiol undecylate, Estradiol valerate, Estramustine, Estramustine phosphate, Estrapronicate, Estrazinol, Estriol, Estriol succinate, Estriol tripropionate, Estrofurate, Estromustine, Estrone, Estrone acetate, Estrone cyanate, Estrone sulfate, Estrone tetraacetylglucoside, Estropipate, Etamestrol, Ethinylestradiol, Ethinylestradiol 3-isopropylsulfonate, Etynodiol diacetate, FERb 033, Fenestrel, Fosfestrol, Furostilbestrol, Hexestrol, Hexestrol diacetate, Hexestrol dicaprylate, Hexestrol diphosphate, Hexestrol dipropionate, Hydroxyestrone diacetate, Liquiritigenin, Mestranol, Methallenestril, Methestrol, Methestrol dipropionate, Moxestrol, Nilestriol, Orestrate, Polyestradiol phosphate, PPT, Promestriene, Promethoestrol, Quinestradol, Quinestrol, β-Sitosterol, or WAY-200,070.
74. The composition according to embodiment 55, wherein the androgen agonist is a full androgen agonist or a partial androgen agonist.
75. The composition according to embodiment 55, wherein the androgen agonist is 1-Androstenediol, 1-Testosterone, 1,4-Androstenedione, 4-Androstenediol, 4-Chlordehydromethyltestosterone, 4-Hydroxytestosterone, 5-Androstenediol, 5α-Androst-1-ene-3,17-dione, 11-Ketotestosterone, 17-((1-Oxoheptyl)oxy)androstan-3-one, 17-((1-Oxopentypoxy)androstan-3-one, 17-(1-Oxopropoxy)androstan-3-one, 17β-Hydroxyandrost-2-ene-2-carbonitrile, 19-Norandrostenediol, 19-Norandrostenedione, 19-Norandrosterone, 19-Nordehydrotestosterone, Adrenosterone, Androisoxazole, Androstanolone, Androstenediol, Androstenedione, Androsterone, Bolandiol, Bolasterone, Bolazine, Boldenone, Boldione, Bolenol, Bolmantalate, Calusterone, Clostebol, Cloxotestosterone, Danazol, DHEA, Dehydroepiandrosterone sulfate (DHEA-S), Desogestrel, Desoxymethyltestosterone, DHT, Drostanolone, Enestebol, Epitiostanol, Ethisterone, Ethyldienolone, Ethylestrenol, Fluoxymesterone, Formebolone, Furazabol, Hexabolan, Human Growth Hormone (HGH), Hydroxystenozole, Levonorgestrel, Mebolazine, Mepitiostane, Mesabolone, Mestanolone, Mesterolone, Metandienone, Methandrostenolone, Metenolone, Metenolone acetate, Metenolone enanthate, Methandriol, Methandriol propionate, Methasterone, Methylestrenolone, Methyltestosterone, Metribolone, Mibolerone, Nandrolone, Nandrolone caproate, Nandrolone cypionate, Nandrolone cyclohexane carboxylate, Nandrolone cyclohexylpropionate, Nandrolone cyclotate, Nandrolone decanoate, Nandrolone furylpropionate, Nandrolone hexyloxyphenylpropionate, Nandrolone hydrogen succinate, Nandrolone laurate, Nandrolone phenylpropionate, Nandrolone propionate, Nandrolone undecyclate, Norbolethone, Norclostebol, Norethandrolone, Norethisterone, Norgestrel, Oxabolone, Oxabolone cypionate, Oxandrolone, Oxendolone, Oxymesterone, Oxymetholone, Penmesterol, Propetandrol, Quinbolone, Roxibolone, Silandrone, Stanozolol, Stenbolone, Stenbolone acetate, Testolactone, Testosterone, Testosterone enanthate, Testosterone ketolaurate, Testosterone nicotinate, Testosterone propionate, Testosterone undecanoate, THG, Thiomesterone, Tibolone, Tiomesterone, Trenbolone, Trenbolone acetate, Trestolone, or any combination thereof.
76. The composition according to embodiment 55, wherein the SARM includes AC-262,356, Andarine, BMS-564,929, Enobosarm, LGD-2226, LGD-3303, S-23, S-40503, or any combination thereof.
77. The composition according to embodiment 55, wherein the androgen antagonist is a full androgen antagonist or a partial androgen antagonist.
78. The composition according to embodiment 55, wherein the androgen antagonist includes ARN-509, Benorterone, Bicalutamide, BMS-641,988, BOMT, Canrenoic acid, Canrenone, Chlormadinone acetate, Cimetidine, Cioteronel, Cyproterone, Cyproterone acetate, Delanterone, Dienogeste, Enzalutamide, Epitestosterone, Flutamide, Galeterone, Hydroxyflutamide, Hydroxyprogesterone caproate, Inocoterone, Ketoconazole, Megestrol acetate, Metogest, Mifepristone, Nilutamide, Nomegestrol, Nordinone, Norgestimate, Osaterone, Oxendolone, Potassium canrenoate, Progesterone, R2956, Rosterolone, RU-58642, RU-58841, Spironolactone, Topterone, or Zanoterone.
79. The composition according to any one of embodiments 1-78, wherein the pharmaceutical composition reduces an unwanted side.
80. The composition according to embodiment 79, wherein the unwanted side includes sedation, cognitive fogging, dizziness, drowsiness, postural hypertension, coordination problems, weakness, tremors, respiratory depression, psychotropic effects, sleep disturbances, unwanted waitfulness, CNS stimulation, weight gain, appetite change, change in sexual function, constipation, dry mouth, gut erosion, gastric ulcerations, renal inflammation, cardiovascular hypertension, cardiovascular stimulation, hyperchlimina, not going into public, chest pain, stress incontinence, or any combination thereof.
81. The composition according to any one of embodiments 1-80, wherein modulating activity of a hormone reduces a symptom associated with a gender-biased immune disorder.
82. The composition according to embodiment 81, wherein modulating activity of a hormone reduces a symptom associated with a gender-biased immune disorder by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%.
83. The composition according to embodiment 81, wherein the symptom includes the frequency of a symptom, the severity of a symptom, inflammation, immune problem, drowsiness, fainting, nausea, fatigue, fever and high body temperature, extreme sensitivity to cold, malaise, burning pain, abdominal cramping, abdominal swelling, abdominal tenderness, abdominal pain, muscle tone loss, muscle cramping, muscle weakness, muscle stiffness, muscle swelling, muscle tenderness, muscle pain, joint weakness, joint stiffness, joint swelling, joint tenderness, joint inflammation, joint pain, arthritis, temporary loss of cognitive abilities, cognitive fogging, dizziness, drowsiness, chronic anxiety, chronic bloating, chronic constipation, chronic depression, chronic diarrhea, chronic fatigue syndrome (CFS), chronic headache, chronic indigestion, chronic insomnia, chronic irritability, chronic migraine, chronic nausea, chronic pain, malar blush, disoid rash, alopecia, Raynaud phenomenon, livedo reticularis, panniculitis (lupus profundus), bullous lesions, vasculitic purpura, telangiectasias, urticaria, photosensitivity, a skin lesion, skin disorder, nasal inflammation, hemorrhoids, rectal bleeding, blood in stool, constipation, intestinal bleeding, intestinal obstruction, acid reflux, diarrhea, digestive problems, indigestion, fistula, mesenteric vasculitis, bowel infarction, flatulence, excess gas, hematuria, proteinuria, uremia, nephritis, glomerulonephritis, edema, bloating, weight gain, hyperlipidemia, high cholesterol, pericarditis, hypertension, vasculitis, myocarditis, endocarditis, atherosclerosis, dyspnea, angina, encephalopathy, cerebritis, headache, migraine, cognitive dysfunction, mood disorder, confusion, insomnia, irritability, depression, memory loss, cerebrovascular disease, seizures, polyneuropathy, anxiety disorder, agitation, psychosis, personality disorder, intracranial pressure, papilledema, nerve paresis, paranoia, hallucination, Guillain-Barré syndrome, aseptic meningitis, autonomic disorder, demyelinating syndrome, mononeuropathy, movement disorder, myasthenia gravis, myelopathy, cranial neuropathy, plexopathy, aseptic meningitis, myelopathy, optic neuropathy, a demyelinating disorder, myelitis, paraparesis, ischemia, transient ischemic attack, stroke, pleuritis, pleurisy, pleural effusion, pneumonitis, hemoptysis, chronic diffuse interstitial lung disease, pulmonary hypertension, low or high blood pressure, pulmonary emboli, pulmonary hemorrhage, shrinking lung syndrome, anemia, thrombocytopenia, leucopenia, lymphopenia, phlebitis, menstrual problem, miscarriage, tissue degeneration, ulcer, dry eye, vision loss, peritonitis, gland or lymph problem, hormonal imbalance, pancreatitis, hormonal imbalance, infertility or reduced sex drive (low libido), low blood sugar level, high blood sugar level, blood sugar changes, jaundice, abnormal growths, abnormal tissue formation, polyps, abscess, hair loss, general infection, bacterial infection, fungal infection, parasitic infection, and/or yeast infection, an increase in the size of an organ or tissue, or the destruction of an organ or tissue, or any combination thereof.
84. The composition according to any one of embodiments 1-83, wherein the composition is formulated for inhalatory administration.
85. The composition according to any one of any one of embodiments 1-84, wherein the composition is formulated for oral administration.
86. The composition according to any one of embodiments 1-85, wherein the composition includes a progesterone agonist and a SPRM.
87. The composition according to any one of embodiments 1-85, wherein the composition includes a progesterone agonist and an estrogen antagonist.
88. The composition according to any one of embodiments 1-85, wherein the composition includes a progesterone agonist and a SERM.
89. The composition according to any one of embodiments 1-85, wherein the composition includes a progesterone agonist and an estrogen biosynthesis enzyme inhibitor.
90. The composition according to any one of embodiments 1-85, wherein the composition includes a progesterone agonist and an androgen agonist.
91. The composition according to any one of embodiments 1-85, wherein the composition includes a progesterone agonist and a SARM.
92. The composition according to any one of embodiments 1-85, wherein the composition includes an estrogen agonist and a progesterone antagonist.
93. The composition according to any one of embodiments 1-85, wherein the composition includes an estrogen agonist and a SPRM.
94. The composition according to any one of embodiments 1-85, wherein the composition includes an estrogen agonist and an androgen antagonist.
95. The composition according to any one of embodiments 1-85, wherein the composition includes an estrogen agonist and a SARM.
96. A method of treating a gender-biased disorder, the method comprising the step of administering a composition according to embodiments 1-95 to an individual, wherein administration reduces a symptom associated with the gender-biased immune disorder.
97. Use of a composition according to any one of embodiments 1-95 in the manufacture of a medicament for the treatment of a gender-biased immune disorder.
98. Use of a composition according to any one of embodiments 1-95 in the treatment of a gender-biased immune disorder.
99. The method according to embodiment 96 or use according to embodiment 97 or 98, wherein administration of the composition reduces the occurrence of an unwanted side.
100. The method or use according to embodiment 99, wherein the unwanted side effect includes masculinization, increased muscle mass, increased fat deposition, menorrhea, increased hair growth on face, torso, and/or extremities, feminization, mammary gland development, cancer, growth hormone effects, diabetes, mood swings, hair loss or growth on face, torso, and/or extremities, change in voice resonance or pitch, reduced sexual desire, reduced sexual arousal, reduced sexual orgasm, erectile dysfunction, impotence, infertility, or any combination thereof.
101. The method or use according to any one of embodiments 96-100, wherein the gender-biased immune disorder is a Th1-based immune disorder.
102. The method or use according to any one of embodiments 96-100, wherein the gender-biased immune disorder is a Th2-based immune disorder.
103. The method or use according to any one of embodiments 96-102, wherein the gender-biased immune disorder is an autoimmune disorder.
104. The method or use according to embodiment 101, wherein the Th1-based immune disorder is rheumatoid arthritis, reactive arthritis, multiple sclerosis, Chagus disease, Crohn's disease, psoriasis, psoriatic arthritis, juvenile-onset rheumatoid arthritis, uvetis, age-related macular degeneration, pandemic flu, respiratory syncytical virus, cystic fibrosis, genital herpes, sepsis, septic shock, dengue hemorrhagic fever, type 1 diabetes, endometrosis, or prostatis
105. The method or use according to embodiment 102, wherein the Th2-based immune disorder is asthma, allergic asthma, systemic lupus erythematosus, cutaneous lupus, atopic disease, eczema, allergic rhinitis, ulcerative colitis, scleroderma, or sarcoidosis.
106. The method or use according to any one of embodiments 96-100, wherein the gender-biased immune disorder is an acute disseminated encephalomyelitis (ADEM), an Addison's disease, an allergy, allergic rhinitis, an Alzheimer's disease, alopecia areata, amyotrophic lateral sclerosis, anemia, ankylosing spondylitis, an anti-GBM nephritis, an anti-TBM nephritis, an anti-phospholipid antibody syndrome (APS), aplastic anemia, an arthritis, an asthma, atopic allergy, an autoimmune deficiency syndrome (AIDS), an autoimmune hemolytic anemia, an autoimmune hepatitis, an autoimmune inner ear disease, an autoimmune lymphoproliferative syndrome (ALPS), a Balo disease, a Barrett's esophagus, a Behcet's disease, a Berger's disease (IgA nephropathy), a bullous pemphigoid, a bursitis, a cardiomyopathy, a celiac disease, a Chagas disease, a chronic obstructive pulmonary disease (COPD), Churg Strauss syndrome, cicatricial pemphigoid, Cogan's syndrome, cold agglutunin disease, a cranial arteritis, a CREST syndrome, a Cushing's syndrome, a Dego's disease, a dermatitis, a dermatomyositis, a devic disease, a diabetes mellitus type 1 (IDDM), a diabetes mellitus type 2, Dressler's syndrome, eczema, eosinophilic fasciitis, epidermolysis bullosa acquisita, an endometriosis, essential mixed cryoglobulinemia, Evan's syndrome, fibromyalgia, fibrosing alveolitis, a gastrointestinal disorder such as, e.g., digestive dysfunction, diverticulitis, diverticulosis, an irritable bowel disease or an inflammatory bowel disease like Crohn's disease or an ulcerative colitis, a giant cell arteritis, a glomerulonephritis, a Goodpasture's syndrome, a Graves' disease, a Guillain-Barré syndrome (GBS), a Hashimoto's thyroiditis, a hemolytic anemia, a hemorrhoids, a Henoch-Schönlein purpura, a hepatitis, a hiatal hernia, a hidradenitis suppurativa, a Hughes syndrome, an idiopathic adrenal atrophy, an idiopathic pulmonary fibrosis, an idiopathic thrombocytopenia purpura, inflammatory demylinating polyneuropathy, an interstitial cystitis, a Kawasaki's disease, a leaky gut syndrome, a lichen planus, a lupoid hepatitis, a lupus, a Lyme disease, a Meniere's disease, a mixed connective tissue disease, a morphea, a multiple myeloma, a multiple sclerosis (MS), a myasthenia gravis, a myopathy, a myositis, a narcolepsy, a neuromyotonia, an ocular cicatricial pemphigoid, an osteoporosis, a Parkinson's disease, a pars planitis, a pemphigus vulgaris, a pernicious anaemia, a polyglandular autoimmune syndrome, a polymyalgia rheumatic, a polymyositis, a primary biliary cirrhosis, a primary sclerosing cholangitis, a proctitis, a Raynaud's phenomenon, a Reiter's syndrome, a recurrent disseminated encephalomyelitis, a rheumatic fever, a schizophrenia, a scleritis, a scleroderma, a Sjögren's syndrome, a skin disorder such as, e.g., dermatitis, an eczema, a statis dermatitis, a hidradenitis suppurativa, a psoriasis, a rosacea or a sarcoidosis, a sticky blood syndrome, a stiff man syndrome, a Still's disease, a sydenham chorea, a tenosynovitis, a uveitis, a vasculitis, a vitiligo, a Wilson's syndrome, or a transplant rejection include a hyperacute rejection, an acute rejection, or a chronic rejection, as well as, a graft-versus-host-disease.
107. The method or use according to embodiments 106, wherein the arthritis is a monoarthritis, an oligoarthritis, or a polyarthritis like an osteoarthritis, a rheumatoid arthritis, a juvenile idiopathic arthritis, a septic arthritis, a spondyloarthropathy, a gout, a pseudogout, or Still's disease,
108. The method or use according to embodiments 106, wherein the lupus is a discoid lupus erythematosus, a drug-induced lupus erythematosus, a lupus nephritis, a neonatal lupus, a subacute cutaneous lupus erythematosus, or a systemic lupus erythematosus,
109. The method or use according to embodiments 106, wherein the vasculitis is a Buerger's disease, a cerebral vasculitis, a Churg-Strauss arteritis, a cryoglobulinemia, an essential cryoglobulinemic vasculitis, a giant cell arteritis, a Golfers vasculitis, a Henoch-Schönlein purpura, a hypersensitivity vasculitis, a Kawasaki disease, a microscopic polyarteritis/polyangiitis, a polyarteritis nodosa, a polymyalgia rheumatica (PMR), a rheumatoid vasculitis, a Takayasu arteritis, a temporal arteritis, or a Wegener's granulomatosis,
110. The method or use according to any one of embodiments 96-109, wherein the symptom includes the frequency of a symptom, the severity of a symptom, inflammation, immune problem, drowsiness, fainting, nausea, fatigue, fever and high body temperature, extreme sensitivity to cold, malaise, burning pain, abdominal cramping, abdominal swelling, abdominal tenderness, abdominal pain, muscle tone loss, muscle cramping, muscle weakness, muscle stiffness, muscle swelling, muscle tenderness, muscle pain, joint weakness, joint stiffness, joint swelling, joint tenderness, joint inflammation, joint pain, arthritis, temporary loss of cognitive abilities, cognitive fogging, dizziness, drowsiness, chronic anxiety, chronic bloating, chronic constipation, chronic depression, chronic diarrhea, chronic fatigue syndrome (CFS), chronic headache, chronic indigestion, chronic insomnia, chronic irritability, chronic migraine, chronic nausea, chronic pain, malar blush, disoid rash, alopecia, Raynaud phenomenon, livedo reticularis, panniculitis (lupus profundus), bullous lesions, vasculitic purpura, telangiectasias, urticaria, photosensitivity, a skin lesion, skin disorder, nasal inflammation, hemorrhoids, rectal bleeding, blood in stool, constipation, intestinal bleeding, intestinal obstruction, acid reflux, diarrhea, digestive problems, indigestion, fistula, mesenteric vasculitis, bowel infarction, flatulence, excess gas, hematuria, proteinuria, uremia, nephritis, glomerulonephritis, edema, bloating, weight gain, hyperlipidemia, high cholesterol, pericarditis, hypertension, vasculitis, myocarditis, endocarditis, atherosclerosis, dyspnea, angina, encephalopathy, cerebritis, headache, migraine, cognitive dysfunction, mood disorder, confusion, insomnia, irritability, depression, memory loss, cerebrovascular disease, seizures, polyneuropathy, anxiety disorder, agitation, psychosis, personality disorder, intracranial pressure, papilledema, nerve paresis, paranoia, hallucination, Guillain-Barré syndrome, aseptic meningitis, autonomic disorder, demyelinating syndrome, mononeuropathy, movement disorder, myasthenia gravis, myelopathy, cranial neuropathy, plexopathy, aseptic meningitis, myelopathy, optic neuropathy, a demyelinating disorder, myelitis, paraparesis, ischemia, transient ischemic attack, stroke, pleuritis, pleurisy, pleural effusion, pneumonitis, hemoptysis, chronic diffuse interstitial lung disease, pulmonary hypertension, low or high blood pressure, pulmonary emboli, pulmonary hemorrhage, shrinking lung syndrome, anemia, thrombocytopenia, leucopenia, lymphopenia, phlebitis, menstrual problem, miscarriage, tissue degeneration, ulcer, dry eye, vision loss, peritonitis, gland or lymph problem, hormonal imbalance, pancreatitis, hormonal imbalance, infertility or reduced sex drive (low libido), low blood sugar level, high blood sugar level, blood sugar changes, jaundice, abnormal growths, abnormal tissue formation, polyps, abscess, hair loss, general infection, bacterial infection, fungal infection, parasitic infection, and/or yeast infection, an increase in the size of an organ or tissue, or the destruction of an organ or tissue, or any combination thereof.
111. The method or use according to embodiment 110, wherein the inflammation symptom is edema, hyperemia, erythema, bruising, tenderness, stiffness, swollenness, fever, a chill, congestion of the respiratory tract including nose, and bronchi, congestion of a sinus, a breathing problem, fluid retention, a blood clot, a loss of appetite, an increased heart rate, a formation of granulomas, fibrinous, pus, or non-viscous serous fluid, a formation of an ulcer, pain, or any combination thereof.
112. The method or use according to any one of embodiments 96-111, wherein the composition includes a therapeutically effective amount of a therapeutic compound capable of modulating activity of a hormone.
113. The method or use according to embodiment 112, wherein the therapeutically effective amount of the therapeutic compound is in the range of about 0.001 mg/kg/day to about 10 mg/kg/day.
114. The method or use according to embodiment 112, wherein the therapeutically effective amount of the therapeutic compound is in the range of about 0.01 mg/kg/day to about 100 mg/kg/day.
115. The method or use according to embodiment 112, wherein the therapeutically effective amount of the therapeutic compound is in the range of about 0.1 mg/day to about 800 mg/day.
116. The method or use according to embodiment 112, wherein the therapeutically effective amount of the therapeutic compound is in the range of about 1 mg/day to about 8,000 mg/day.
117. The method or use according to any one of embodiments 96-116, wherein the composition is administered for one or more cycles.
118. The method or use according to embodiment 117, wherein the one or more cycles is about 1 cycle to about 2 cycles, about 1 cycle to about 3 cycles, about 1 cycle to about 4 cycles, about 1 cycle to about 5 cycles, about 1 cycle to about 6 cycles, about 1 cycle to about 7 cycles, about 1 cycle to about 8 cycles, about 1 cycle to about 9 cycles, about 1 cycle to about 10 cycles, about 1 cycle to about 11 cycles, about 1 cycle to about 12 cycles, about 1 cycle to about 13 cycles, about 1 cycle to about 14 cycles, or about 1 cycle to about 15 cycles.
119. The method or use according to embodiment 117 or embodiment 118, wherein the one or more cycles each comprise administering the composition to an individual for a first period of time followed by a second period of time where the composition disclosed herein is not administered to the individual.
120. The method or use according to embodiment 119, wherein the one or more cycles is at least 2 cycles, at least 3 cycles, at least 4 cycles, at least 5 cycles, at least 6 cycles, at least 7 cycles, at least 8 cycles, at least 9 cycles, at least 10 cycles, at least 11 cycles, at least 12 cycles, at least 13 cycles, at least 14 cycles, or at least 15 cycles, or at most 2 cycles, at most 3 cycles, at most 4 cycles, at most 5 cycles, at most 6 cycles, at most 7 cycles, at most 8 cycles, at most 9 cycles, at most 10 cycles, at most 11 cycles, at most 12 cycles, at most 13 cycles, at most 14 cycles, or at most 15 cycles, or about 2 cycles to about 3 cycles, about 2 cycles to about 4 cycles, about 2 cycles to about 5 cycles, about 2 cycles to about 6 cycles, about 2 cycles to about 7 cycles, about 2 cycles to about 8 cycles, about 2 cycles to about 9 cycles, about 2 cycles to about 10 cycles, about 2 cycles to about 11 cycles, about 2 cycles to about 12 cycles, about 2 cycles to about 13 cycles, about 2 cycles to about 14 cycles, about 2 cycles to about 15 cycles, about 3 cycles to about 4 cycles, about 3 cycles to about 5 cycles, about 3 cycles to about 6 cycles, about 3 cycles to about 7 cycles, about 3 cycles to about 8 cycles, about 3 cycles to about 9 cycles, about 3 cycles to about 10 cycles, about 3 cycles to about 11 cycles, about 3 cycles to about 12 cycles, about 3 cycles to about 13 cycles, about 3 cycles to about 14 cycles, about 3 cycles to about 15 cycles, about 4 cycles to about 5 cycles, about 4 cycles to about 6 cycles, about 4 cycles to about 7 cycles, about 4 cycles to about 8 cycles, about 4 cycles to about 9 cycles, about 4 cycles to about 10 cycles, about 4 cycles to about 11 cycles, about 4 cycles to about 12 cycles, about 4 cycles to about 13 cycles, about 4 cycles to about 14 cycles, about 4 cycles to about 15 cycles, about 5 cycles to about 6 cycles, about 5 cycles to about 7 cycles, about 5 cycles to about 8 cycles, about 5 cycles to about 9 cycles, about 5 cycles to about 10 cycles, about 5 cycles to about 11 cycles, about 5 cycles to about 12 cycles, about 5 cycles to about 13 cycles, about 5 cycles to about 14 cycles, about 5 cycles to about 15 cycles, about 6 cycles to about 7 cycles, about 6 cycles to about 8 cycles, about 6 cycles to about 9 cycles, about 6 cycles to about 10 cycles, about 6 cycles to about 11 cycles, about 6 cycles to about 12 cycles, about 6 cycles to about 13 cycles, about 6 cycles to about 14 cycles, about 6 cycles to about 15 cycles, about 7 cycles to about 8 cycles, about 7 cycles to about 9 cycles, about 7 cycles to about 10 cycles, about 7 cycles to about 11 cycles, about 7 cycles to about 12 cycles, about 7 cycles to about 13 cycles, about 7 cycles to about 14 cycles, about 7 cycles to about 15 cycles, about 8 cycles to about 9 cycles, about 8 cycles to about 10 cycles, about 8 cycles to about 11 cycles, about 8 cycles to about 12 cycles, about 8 cycles to about 13 cycles, about 8 cycles to about 14 cycles, about 8 cycles to about 15 cycles, about 9 cycles to about 10 cycles, about 9 cycles to about 11 cycles, about 9 cycles to about 12 cycles, about 9 cycles to about 13 cycles, about 9 cycles to about 14 cycles, about 9 cycles to about 15 cycles, about 10 cycles to about 11 cycles, about 10 cycles to about 12 cycles, about 10 cycles to about 13 cycles, about 10 cycles to about 14 cycles, about 10 cycles to about 15 cycles, about 11 cycles to about 12 cycles, about 11 cycles to about 13 cycles, about 11 cycles to about 14 cycles, about 11 cycles to about 15 cycles, about 12 cycles to about 13 cycles, about 12 cycles to about 14 cycles, about 12 cycles to about 15 cycles, about 13 cycles to about 14 cycles, about 13 cycles to about 15 cycles, or about 14 cycles to about 15 cycles.
121. The method or use according to embodiment 119 and embodiment 120, wherein the first period of time is about 1 day, about 2 days, about 3 days, about 4 days, about 5 days, about 6 days, about 7 days, about 8 days, about 9 days, about 10 days, about 11 days, about 12 days, about 13 days, about 14 days, about 3 weeks, about 4 weeks, about 5 weeks, about 6 weeks, about 7 weeks, about 8 weeks, about 9 weeks, about 10 weeks, about 11 weeks, or about 12 weeks, or at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 3 weeks, at least 4 weeks, at least 5 weeks, at least 6 weeks, at least 7 weeks, at least 8 weeks, at least 9 weeks, at least 10 weeks, at least 11 weeks, or at least 12 weeks, or at most 1 day, at most 2 days, at most 3 days, at most 4 days, at most 5 days, at most 6 days, at most 7 days, at most 8 days, at most 9 days, at most 10 days, at most 11 days, at most 12 days, at most 13 days, at most 14 days, at most 3 weeks, at most 4 weeks, at most 5 weeks, at most 6 weeks, at most 7 weeks, at most 8 weeks, at most 9 weeks, at most 10 weeks, at most 11 weeks, or at most 12 weeks, about 1 day to about 3 days, about 1 day to about 5 days, about 1 day to about 7 days, about 1 day to about 9 days, about 1 day to about 10 days, about 1 day to about 12 days, about 1 day to about 14 days, about 1 day to about 15 days, about 1 day to about 18 days, about 1 day to about 21 days, about 1 day to about 24 days, about 1 day to about 27 days, about 1 day to about 28 days, about 3 days to about 5 days, about 3 days to about 7 days, about 3 days to about 9 days, about 3 days to about 10 days, about 3 days to about 12 days, about 3 days to about 14 days, about 3 days to about 15 days, about 3 days to about 18 days, about 3 days to about 21 days, about 3 days to about 24 days, about 3 days to about 27 days, about 3 days to about 28 days, about 5 days to about 7 days, about 5 days to about 9 days, about 5 days to about 10 days, about 5 days to about 12 days, about 5 days to about 14 days, about 5 days to about 15 days, about 5 days to about 18 days, about 5 days to about 21 days, about 5 days to about 24 days, about 5 days to about 27 days, about 5 days to about 28 days, about 7 days to about 9 days, about 7 days to about 10 days, about 7 days to about 12 days, about 7 days to about 14 days, about 7 days to about 17 days, about 7 days to about 18 days, about 7 days to about 21 days, about 7 days to about 24 days, about 7 days to about 27 days, or about 7 days to about 28 days, or about 1 week to about 2 weeks, about 1 week to about 3 weeks, about 1 week to about 4 weeks, about 1 week to about 5 weeks, about 1 week to about 6 weeks, about 1 week to about 7 weeks, about 1 week to about 8 weeks, about 1 week to about 9 weeks, about 1 week to about 10 weeks, about 1 week to about 11 weeks, about 1 week to about 12 weeks, about 2 weeks to about 3 weeks, about 2 weeks to about 4 weeks, about 2 weeks to about 5 weeks, about 2 weeks to about 6 weeks, about 2 weeks to about 7 weeks, about 2 weeks to about 8 weeks, about 2 weeks to about 9 weeks, about 2 weeks to about 10 weeks, about 2 weeks to about 11 weeks, about 2 weeks to about 12 weeks, about 3 weeks to about 4 weeks, about 3 weeks to about 5 weeks, about 3 weeks to about 6 weeks, about 3 weeks to about 7 weeks, about 3 weeks to about 8 weeks, about 3 weeks to about 9 weeks, about 3 weeks to about 10 weeks, about 3 weeks to about 11 weeks, about 3 weeks to about 12 weeks, about 4 weeks to about 5 weeks, about 4 weeks to about 6 weeks, about 4 weeks to about 7 weeks, about 4 weeks to about 8 weeks, about 4 weeks to about 9 weeks, about 4 weeks to about 10 weeks, about 4 weeks to about 11 weeks, about 4 weeks to about 12 weeks, about 5 weeks to about 6 weeks, about 5 weeks to about 7 weeks, about 5 weeks to about 8 weeks, about 5 weeks to about 9 weeks, about 5 weeks to about 10 weeks, about 5 weeks to about 11 weeks, about 5 weeks to about 12 weeks, about 6 weeks to about 7 weeks, about 6 weeks to about 8 weeks, about 6 weeks to about 9 weeks, about 6 weeks to about 10 weeks, about 6 weeks to about 11 weeks, about 6 weeks to about 12 weeks, about 7 weeks to about 8 weeks, about 7 weeks to about 9 weeks, about 7 weeks to about 10 weeks, about 7 weeks to about 11 weeks, about 7 weeks to about 12 weeks, about 8 weeks to about 9 weeks, about 8 weeks to about 10 weeks, about 8 weeks to about 11 weeks, about 8 weeks to about 12 weeks, about 9 weeks to about 10 weeks, about 9 weeks to about 11 weeks, about 9 weeks to about 12 weeks, about 10 weeks to about 11 weeks, about 10 weeks to about 12 weeks, or about 11 weeks to about 12 weeks.
122. The method or use according to any one of embodiments 119-121, wherein the second period of time is about 1 day, about 2 days, about 3 days, about 4 days, about 5 days, about 6 days, about 7 days, about 8 days, about 9 days, about 10 days, about 11 days, about 12 days, about 13 days, about 14 days, about 3 weeks, about 4 weeks, about 5 weeks, about 6 weeks, about 7 weeks, about 8 weeks, about 9 weeks, about 10 weeks, about 11 weeks, about 12 weeks, about 1 month, about 2 months, about 3 months, about 4 months, about 5 months, about 6 months, about 7 months, about 8 months, about 9 months, about 10 months, about 11 months, or about 12 months, at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 1 month, at least 2 months, at least 3 months, at least 4 months, at least 5 months, at least 6 months, at least 7 months, at least 8 months, at least 9 months, at least 10 months, at least 11 months, or at least 12 months, or at most 1 day, at most 2 days, at most 3 days, at most 4 days, at most 5 days, at most 6 days, at most 7 days, at most 8 days, at most 9 days, at most 10 days, at most 11 days, at most 12 days, at most 13 days, at most 14 days, at most 3 weeks, at most 4 weeks, at most 5 weeks, at most 6 weeks, at most 7 weeks, at most 8 weeks, at most 9 weeks, at most 10 weeks, at most 11 weeks, at most 12 weeks, at most 1 month, at most 2 months, at most 3 months, at most 4 months, at most 5 months, at most 6 months, at most 7 months, at most 8 months, at most 9 months, at most 10 months, at most 11 months, or at most 12 months, or about 1 day to about 3 days, about 1 day to about 5 days, about 1 day to about 7 days, about 1 day to about 9 days, about 1 day to about 10 days, about 1 day to about 12 days, about 1 day to about 14 days, about 1 day to about 15 days, about 1 day to about 18 days, about 1 day to about 21 days, about 1 day to about 24 days, about 1 day to about 27 days, about 1 day to about 28 days, about 3 days to about 5 days, about 3 days to about 7 days, about 3 days to about 9 days, about 3 days to about 10 days, about 3 days to about 12 days, about 3 days to about 14 days, about 3 days to about 15 days, about 3 days to about 18 days, about 3 days to about 21 days, about 3 days to about 24 days, about 3 days to about 27 days, about 3 days to about 28 days, about 5 days to about 7 days, about 5 days to about 9 days, about 5 days to about 10 days, about 5 days to about 12 days, about 5 days to about 14 days, about 5 days to about 15 days, about 5 days to about 18 days, about 5 days to about 21 days, about 5 days to about 24 days, about 5 days to about 27 days, about 5 days to about 28 days, about 7 days to about 9 days, about 7 days to about 10 days, about 7 days to about 12 days, about 7 days to about 14 days, about 7 days to about 17 days, about 7 days to about 18 days, about 7 days to about 21 days, about 7 days to about 24 days, about 7 days to about 27 days, or about 7 days to about 28 days, or about 1 week to about 2 weeks, about 1 week to about 3 weeks, about 1 week to about 4 weeks, about 1 week to about 5 weeks, about 1 week to about 6 weeks, about 1 week to about 7 weeks, about 1 week to about 8 weeks, about 1 week to about 9 weeks, about 1 week to about 10 weeks, about 1 week to about 11 weeks, about 1 week to about 12 weeks, about 2 weeks to about 3 weeks, about 2 weeks to about 4 weeks, about 2 weeks to about 5 weeks, about 2 weeks to about 6 weeks, about 2 weeks to about 7 weeks, about 2 weeks to about 8 weeks, about 2 weeks to about 9 weeks, about 2 weeks to about 10 weeks, about 2 weeks to about 11 weeks, about 2 weeks to about 12 weeks, about 3 weeks to about 4 weeks, about 3 weeks to about 5 weeks, about 3 weeks to about 6 weeks, about 3 weeks to about 7 weeks, about 3 weeks to about 8 weeks, about 3 weeks to about 9 weeks, about 3 weeks to about 10 weeks, about 3 weeks to about 11 weeks, about 3 weeks to about 12 weeks, about 4 weeks to about 5 weeks, about 4 weeks to about 6 weeks, about 4 weeks to about 7 weeks, about 4 weeks to about 8 weeks, about 4 weeks to about 9 weeks, about 4 weeks to about 10 weeks, about 4 weeks to about 11 weeks, about 4 weeks to about 12 weeks, about 5 weeks to about 6 weeks, about 5 weeks to about 7 weeks, about 5 weeks to about 8 weeks, about 5 weeks to about 9 weeks, about 5 weeks to about 10 weeks, about 5 weeks to about 11 weeks, about 5 weeks to about 12 weeks, about 6 weeks to about 7 weeks, about 6 weeks to about 8 weeks, about 6 weeks to about 9 weeks, about 6 weeks to about 10 weeks, about 6 weeks to about 11 weeks, about 6 weeks to about 12 weeks, about 7 weeks to about 8 weeks, about 7 weeks to about 9 weeks, about 7 weeks to about 10 weeks, about 7 weeks to about 11 weeks, about 7 weeks to about 12 weeks, about 8 weeks to about 9 weeks, about 8 weeks to about 10 weeks, about 8 weeks to about 11 weeks, about 8 weeks to about 12 weeks, about 9 weeks to about 10 weeks, about 9 weeks to about 11 weeks, about 9 weeks to about 12 weeks, about 10 weeks to about 11 weeks, about 10 weeks to about 12 weeks, or about 11 weeks to about 12 weeks, or about 1 month to about 2 months, about 1 month to about 3 months, about 1 month to about 4 months, about 1 month to about 5 months, about 1 month to about 6 months, about 1 month to about 7 months, about 1 month to about 8 months, about 1 month to about 9 months, about 1 month to about 10 months, about 1 month to about 11 months, about 1 month to about 12 months, about 2 months to about 3 months, about 2 months to about 4 months, about 2 months to about 5 months, about 2 months to about 6 months, about 2 months to about 7 months, about 2 months to about 8 months, about 2 months to about 9 months, about 2 months to about 10 months, about 2 months to about 11 months, about 2 months to about 12 months, about 3 months to about 4 months, about 3 months to about 5 months, about 3 months to about 6 months, about 3 months to about 7 months, about 3 months to about 8 months, about 3 months to about 9 months, about 3 months to about 10 months, about 3 months to about 11 months, about 3 months to about 12 months, about 4 months to about 5 months, about 4 months to about 6 months, about 4 months to about 7 months, about 4 months to about 8 months, about 4 months to about 9 months, about 4 months to about 10 months, about 4 months to about 11 months, about 4 months to about 12 months, about 5 months to about 6 months, about 5 months to about 7 months, about 5 months to about 8 months, about 5 months to about 9 months, about 5 months to about 10 months, about 5 months to about 11 months, about 5 months to about 12 months, about 6 months to about 7 months, about 6 months to about 8 months, about 6 months to about 9 months, about 6 months to about 10 months, about 6 months to about 11 months, about 6 months to about 12 months, about 7 months to about 8 months, about 7 months to about 9 months, about 7 months to about 10 months, about 7 months to about 11 months, about 7 months to about 12 months, about 8 months to about 9 months, about 8 months to about 10 months, about 8 months to about 11 months, about 8 months to about 12 months, about 9 months to about 10 months, about 9 months to about 11 months, about 9 months to about 12 months, about 10 months to about 11 months, about 10 months to about 12 months, or about 11 months to about 12 months.
123. The method or use according to any one of embodiments 96-122, wherein the individual is a female, a male or either.
124. The method or use according to any one of embodiments 96-122, wherein the gender-biased immune disorder primarily afflicts females.
125. The method or use according to embodiment 124, wherein the gender-biased immune disorder is Hashimoto's thyroiditis, systemic lupus erythematosus (SLE), Sjogren syndrome, Graves' disease, autoimmune hepatitis, rheumatoid arthritis, osteoarthritis, osteoporosis, chronic fatigue syndrome, fibromyalgia, lupus, irritable bowel syndrome, cataracts, asthma, interstitial cystitis, Thrombocytopenia purpura, myasthenia gravis, multiple sclerosis, systemic sclerosis, or dermatomyositis
126. The method or use according to any one of embodiments 96-122, wherein the gender-biased immune disorder primarily afflicts males.
127. The method or use according to embodiment 126, wherein the gender-biased immune disorder is primary sclerosing cholangitis, ankylosing spondylitis, myocarditis, dilated cardiomyopathy, pernicious anemia, type 1 diabetes mellitus, gastritis, Wegener's granulomatosis, idiopathic pulmonary fibrosis, Crohn's disease, or psoriasis.

### EXAMPLES

The following non-limiting examples are provided for illustrative purposes only in order to facilitate a more complete understanding of representative embodiments now contemplated. These examples should not be construed to limit any of the embodiments described in the present specification, including those pertaining to the compounds, pharmaceutical compositions, methods or uses of treating a gender-biased immune disorder.

### Example 1

### Testosterone and Progesterone Reduce Antigenicity of SLE Causing Proteins

This example illustrates the therapeutic benefit of a hormone in modulating an antibody response.

Male and female mice (7-9 weeks old) were given a single vaccination with a peptide from the P1/P2 ribosomal protein of the Chagas parasite Trypanosome cruzi, designated the CH47 peptide. These mice were then randomly placed in one of four groups and treated as follows: Group 1 mice, three male mice challenged with a CH47 peptide; Group 2 mice, three male mice not challenged with the CH47 peptide and served the control; Group 3 mice, three female mice challenged the CH47 peptide and Group 4 mice, three female mice not challenged with the CH47 peptide and served as the control.

The results are shown in Table 1. Female mice not challenged with the CH47 peptide already had very high levels of pre-existing specific to CH47 (P1/P2), antigen present (Table 1). This indicates that female mice naturally produce high levels of endogenously P1/P2 like antigens, and supports our hypothesis that all women have low level presentation of self antigens that generates a non-pathogenic level of autoantibodies. When challenged with the CH47 peptide, the amount of antigen present paradoxically when down by half, indicating that challenging is causing an internalisation of antigen, a phenomenon that often occurs in systems which are already close to saturation. This clearly demonstrates that female mice are persistently presenting P1/P2 like antigen at high levels, and suggests the reason that female mice are susceptible to SLE disease.

These data also show that male mice not challenged with the CH47 peptide (Group 3) had a very low level of antigen presentation, indicating that male mice have no or very low endogenous P1/P2 presentation (Table 1). This data indicates that, in contrast to female mice, male mice have no endogenous pre-existing P1/P2 like antigen presentation. Although male mice challenged with CH47 (Groups 1 & 2) had appreciably greater amount of antigen present when compared to Group 3 controls, the increment in response was small regardless of the dose used (Table 1). Again, in contract to female mice, this data reveal that male mice are not capable of processing and/or presenting CH47 to B cells in order to elicit an antibody response. This reflects the clinical situation in humans suffering from SLE, where men have a much lower incidence, related to their low level of self antigen presentation.

TABLE 1
Gender Effect on P1/P2-Specific Antobody Production

Antibody Amount (OD₄₅₀ nm)

| Group | Vaccine only | Vaccine + P1/P2 |
|---|---|---|
| Male | 0.10 | 0.12 |
| Female | 0.89 | 0.46 |

Further analysis of this finding above revealed that independent of immunization, male mice have an intrinsic Th1-like cellular mediated immune response to CH47 (P1/P2) (IFN-g data) with no associated antibody production. For example, male mice from the Control Group exhibit a Th1 immune response as demonstrated by the presence of INF-γ production in the absence of CH47 peptide pre-exposure (Table 2). However, no IgG1 or IgG2a antibodies are present in these control male mice indicating that neither Th2-like nor Th1-like antibodies, respectively, were produced (Tables 4 & 5). In contrast, female mice have an intrinsic Th2-like antibody response to CH47 (P1/P2). For example, female mice from the Control Group exhibited a Th1 immune response as demonstrated by the presence of INF-γ production in the absence of CH47 peptide pre-exposure (Table 2). In addition female mice from the Control Group also produced significant amounts of IgG1 antibodies, but not IgG2a antibodies, indicating that these female mice had high levels of Th2-like antibodies (but not Th1-like antibodies) prior to any exposure to the CH47 peptide (Tables 4 & 5).

After vaccination with the CH47 peptide, males do not change the pattern of that immune response. Thus, male mice from the CH47 Group exhibit a Th1 immune response based on INF-γ production (Table 2), but no Th2-like or Th1-like antibody response based on the lack of IgG1 and IgG2a antibody production (Tables 4 & 5). Female mice from the CH47 Group, on the other hand, exhibit a slightly altered pattern of immune response by showing signs of an incipient cellular response. For example, exhibit a small increase in the Th1 immune response based on INF-γ production (Table 2), an increase in IgG2a antibody production (Table 4), but a drop in IgG1 antibody production (Table 5).

Overall, these data suggest that a likely cause to an immune complex mediated disease like SLE is due to the fact that female mice are pre-primed to respond to an antigen challenge with a Th2-like antibody response, whereas male mice are not. The fact that female mice are persistently presenting P1/P2-like antigens at high levels provides the reason why female are susceptible to SLE (a female-biased immune disorder), but resistant to Chagas disease. Therefore, by “changing” females into males (e.g. adding Progesterone or Testosterone) one can treat a female-biased immune disorder like SLE.

In contrast, a likely cause to a T cell infiltration mediated disease like Chagas disease is due to the fact that male mice are pre-primed to respond to antigen challenge with a Th1-like antibody response, whereas female mice are not. The fact that male mice are constantly providing a Th1-like antibody response against P1/P2-like antigens reveals the reason why male are susceptible to Chagas disease (a male-biased immune disorder), but resistant to SLE. Therefore, by “changing” males into females (e.g. adding Estrogen) one can treat a male-biased immune disorder like Chagas disease.

TABLE 2
Th1-Like Cellular Mediated Immune Response to CH47 Peptide

| | | INF-γ Production (pg/mL) | | | | | |
|---|---|---|---|---|---|---|---|
| Group | Gender | CH47 | | Media | | ConA | |
| Control | Male | 333.5 | 198.5 | 31.3 | 0.0 | 1000 | 0.0 |
| | Female | 59.8 | 13.0 | 37.4 | 6.2 | 1000 | 0.0 |
| CH47 | Male | 189.0 | 112.8 | 32.0 | 0.8 | 1000 | 0.0 |
| | Female | 96.7 | 23.4 | 37.5 | 4.7 | 1000 | 0.0 |

TABLE 3
Total Immune Response

| | | Total Ig (OD_{450 nm}) | | | |
|---|---|---|---|---|---|
| | | 1:100 serum | 1:200 serum | 1:400 serum | 1:800 serum |
| Group | Gender | dilution | dilution | dilution | dilution |
| Control | Male | 0.17 ± 0.02 | 0.09 ± 0.01 | 0.06 ± 0.00 | 0.04 ± 0.00 |
| | Female | 2.16 ± 0.36 | 1.16 ± 0.25 | 0.65 ± 0.14 | 0.33 ± 0.07 |
| CH47 | Male | 0.33 ± 0.01 | 0.11 ± 0.00 | 0.06 ± 0.00 | 0.04 ± 0.00 |
| | Female | 1.76 ± 0.13 | 0.86 ± 0.11 | 0.45 ± 0.06 | 0.23 ± 0.03 |

TABLE 4
Th1-Like Antibody Production Against CH47 Peptide

| | | IgG2a (OD_{450 nm}) | | | |
|---|---|---|---|---|---|
| | | 1:100 serum | 1:200 serum | 1:400 serum | 1:800 serum |
| Group | Gender | dilution | dilution | dilution | dilution |
| Control | Male | 0.00 ± 0.00 | 0.00 ± 0.00 | 0.00 ± 0.00 | −0.01 ± 0.00 |
| | Female | 0.05 ± 0.03 | 0.03 ± 0.02 | 0.01 ± 0.01 | 0.00 ± 0.01 |
| CH47 | Male | 0.00 ± 0.00 | 0.00 ± 0.00 | 0.00 ± 0.00 | 0.00 ± 0.00 |
| | Female | 0.73 ± 0.73 | 0.54 ± 0.54 | 0.33 ± 0.33 | 0.17 ± 0.17 |

TABLE 5
Th2-Like Antibody Production Against CH47 Peptide

| | | IgG1 (OD_{450 nm}) | | | |
|---|---|---|---|---|---|
| | | 1:100 serum | 1:200 serum | 1:400 serum | 1:800 serum |
| Group | Gender | dilution | dilution | dilution | dilution |
| Control | Male | 0.00 ± 0.00 | 0.00 ± 0.00 | 0.00 ± 0.00 | −0.01 ± 0.00 |
| | Female | 0.93 ± 0.32 | 0.55 ± 0.20 | 0.30 ± 0.11 | 0.16 ± 0.06 |
| CH47 | Male | 0.05 ± 0.04 | 0.03 ± 0.02 | 0.01 ± 0.01 | 0.00 ± 0.00 |
| | Female | 0.65 ± 0.48 | 0.41 ± 031 | 0.22 ± 0.17 | 0.12 ± 0.09 |

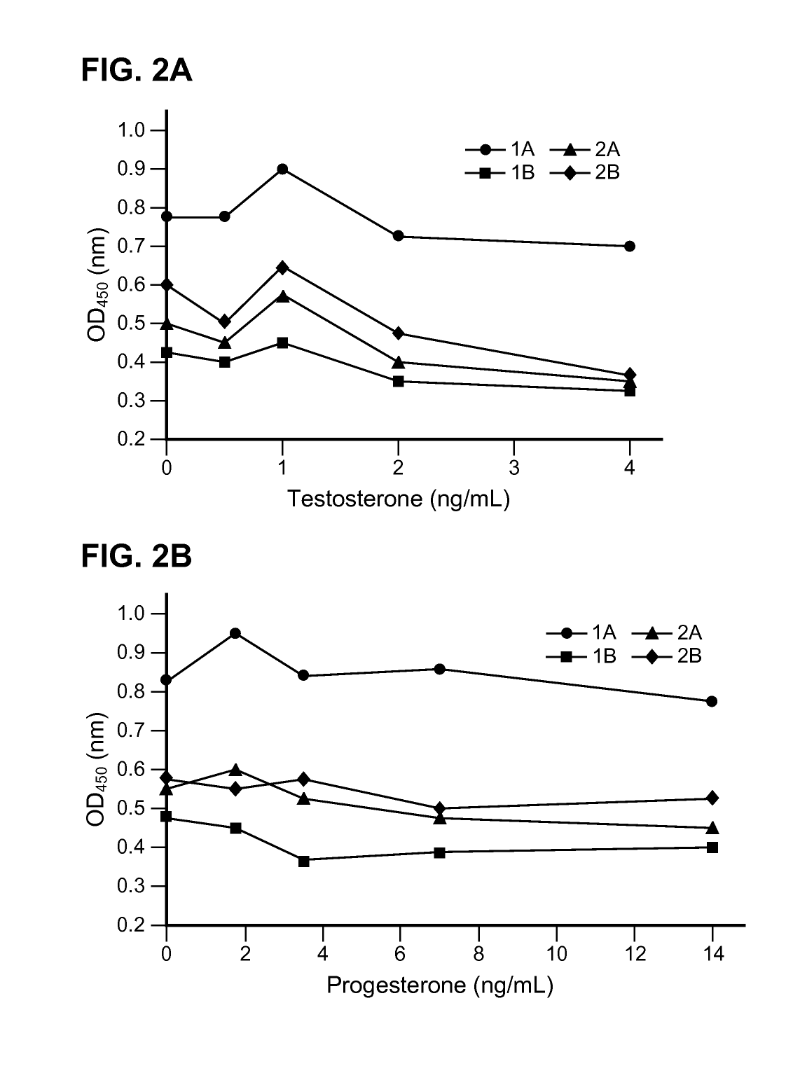

In a second series of experiments, Testosterone (0 ng/mL, 0.5 ng/mL, 1 ng/mL, 2 ng/mL, and 4 ng/mL) or Progesterone (0 ng/mL, 2 ng/mL, 4 ng/mL, 8 ng/mL, and 14 ng/mL) was added to the sera of female mice immunized with a variety of different Chagas peptides (Formulation 1 or 2) that either include (B) or excluded (A) specifically peptide CH47. A decrease in the signal for the CH47 peptide was demonstrated with increased concentration of the hormones (FIGS. 2A & 2B), indicating that these hormones affect the affinity of the antibodies to its target (the region of the P1/P2 protein found in CH47). At low hormone concentrations there was an increase in the antibody signal with a subsequent decrease as the concentration of hormone further increased. Overall, in females, there was a 24.4% decrease in the antibody response with Testosterone and a 26.2% decrease in the IgG response with Progesterone.

In addition, this decrease in antibody response is most likely due to slight activation of a Th1-like response to CH47 challenge since in female mice of the Control Group there is only an IgG1 (Th2-like) response to CH47 (Table 5), whereas in female mice in the CH47 Group there is a drop in the IgG1 (Th2-like) response (Table 5) and a slight increase in the IgG2a (Th1-like) response (Table 4). Furthermore, it appears to be a more IgG related process as IgM is not as susceptible to these changes.

Furthermore, natural responses to CH47 (P1/P2 peptide) are more susceptible to modulation by sex hormones than artificial responses (i.e. by vaccination). For example, the antibody response peak and then drop is more evident in the Control Group, which corresponds to the natural response (i.e. non-challenged induced). This differential could explain why the same antigen is associated with two completely different pathogenic auto-immune responses according to gender.

These results indicate that Testosterone and Progesterone are capable of reducing or inhibiting the affinity of antibodies directed to the CH47 region of the P1/P2 ribosomal protein. The P1/P2 ribosomal protein is highly homologous to human P1/P2 ribosomal proteins and autoantibodies against ribosomal proteins P1/P2 has been frequently detected in SLE patients. The reduction of the antigenicity of SLE causing antigens suggests that Testosterone and/or Progesterone can be beneficial for the treatment of SLE. This is surprising given the fact that Progesterone has been implicated in the precipitation of SLE. Furthermore, the fact that Progesterone and Testosterone both reduce the antigenicity of CH47, after an initial marginal increase, suggest that Estrogen can be causing the exaggerated P1/P2 like antigen presentation.

### Example 2

### Clinical Study in SLE Individuals

A clinical study will be performed involving about 140 individuals suffering from SLE. Participants in the study will need to meet inclusion criteria including being a female at least 18 years old with a negative pregnancy test; being diagnosed with SLE after satisfying four of the criteria listed by the Systemic Lupus International Collaborating Clinics (SLICC) Classification 2012 including at least one clinical criterion and one immunologic criterion; or that the patient has biopsy-proven nephritis compatible with SLE and with ANA or anti-dsDNA antibodies; previouslt suffered at least three SLE flares, each with a SELENA-SLEDAI score greater than or equal to 4. Pregnant or lactating subjects will be excluded from the study, as well as those having been administered Belimumab or Rituximab within the past 6 months and those currently receiving bromocriptine, cyclosporine, ketoconazole, rifamycin, due to drug interaction with progesterone.

Subjects will be administered 200 mg/day of micronized progesterone, with or without 2.5 mg/day of the aromatase inhibitor Letrozole. Both administrative regimes will be administered as a daily oral dose for 14 consecutive days a month (i.e., a two-week on, two-week off dosign regime) for a one year period. Alternatively, the daily oral dose of micronized progesterone can be 300 mg/day for 10 days a month or 100 mg/day for 25 days a month. This therapy is well tolerated, with the only specific side effect being mild and transient drowsiness, an effect minimized by taking the drug at bedtime. The primary endpoints of the study will be to measure changes before and after treatment of the following parameters: 1) blood levels of autoantibodies and estradiol, DHEA and estradiol/DHEA ratio; 2) number of flares and reduction of flare intensity using SELENA-SLEDAI index; 3) amount of corticoid steroid needed to reduce flares; and 4) quality of life scores using EQ-5S Questionnaire. Secondary end points will measure the levels of estrogen and progesterone, autoantibodies and complement in plasma. The effect of treatment on the different variables described (i.e. variation post-treatment minus pre-treatment) will be assessed by Wilcoxon Sign Rank Test with a power of 80% and a two-sided alpha of at least 5%, adjusted as required for multiple variable analysis. Possible associations/correlations between variables significantly affected by the treatment will be assessed using non parametric methods (e.g. McNemar's test, Spearman Rank Correlation Test, etc) to a two-sided alpha of at least 5%.

We believe that the study will show that progesterone with or without an aromatase inhibitor is an affordable treatment for all SLE sufferers with minimal side-effects and would be very easy to be included as the standard of care for SLE.

### Example 3

### Treatment of Gender-Biased Immune Disorders

A 38 year old female complains of puffiness in face, joint and muscle pain, and weight gain. After routine history and physical examination, a physician diagnosis the woman with Hashimoto's thyroiditis, a gender-biased immune disorder. The woman is treated by oral administration a liquid pharmaceutical composition comprising a progesterone agonist as disclosed herein taken once daily. The woman's condition is monitored and after about one week of treatment the woman indicates there is improvement in her health, her face is not as swollen and her joint and muscle pain is less. At one and three month check-ups, the woman indicates that her face is not puffy, she does not suffer joint and muscle pain, and she has experienced some weight loss. This reduction in symptoms in Hashimoto's thyroiditis indicates successful treatment with the pharmaceutical composition disclosed herein. In a similar manner, a pharmaceutical composition any of the other therapeutic compounds disclosed herein, such as, e.g., a SPRM, an estrogen antagonist, a SERM, an estrogen biosynthesis enzyme inhibitor, an androgen agonist, a SARM, or any combination thereof, may be formulated into a pharmaceutical composition and administered to the patient as described above either with or without the progesterone agonist. Furthermore, a pharmaceutical composition any of the other therapeutic compounds disclosed herein may be used to treat other female-biased immune disorders, such as, e.g., osteoarthritis, osteoporosis, chronic fatigue syndrome, fibromyalgia, irritable bowel syndrome, cataracts, interstitial cystitis, Thrombocytopenia purpura, myasthenia gravis, systemic sclerosis, or dermatomyositis.

A 36 year old female complains of fever, joint pains, and fatigue. After routine history and physical examination, a physician diagnosis the woman with Systemic lupus erythematosus, a gender-biased immune disorder. The woman is treated by oral administration a solid pharmaceutical composition comprising a progesterone agonist as disclosed herein taken twice daily. The woman's condition is monitored and after about 1 week of treatment the woman indicates there is improvement in her health, her fever is gone, the pain in her joints is reduced and she is not as tired. At one and three month check-ups, the woman indicates that she continues to have reduced joint pain and does not suffer from fevers or fatigue. This reduction in symptoms in SLE indicates successful treatment with the pharmaceutical composition disclosed herein. In a similar manner, a pharmaceutical composition any of the other therapeutic compounds disclosed herein, such as, e.g., a SPRM, an estrogen antagonist, a SERM, an estrogen biosynthesis enzyme inhibitor, an androgen agonist, a SARM, or any combination thereof, may be formulated into a pharmaceutical composition and administered to the patient as described above either with or without the progesterone agonist. Furthermore, a pharmaceutical composition any of the other therapeutic compounds disclosed herein may be used to treat other female-biased immune disorders, such as, e.g., osteoarthritis, osteoporosis, chronic fatigue syndrome, fibromyalgia, irritable bowel syndrome, cataracts, interstitial cystitis, Thrombocytopenia purpura, myasthenia gravis, systemic sclerosis, or dermatomyositis.

A 29 year old female complains of dye eyes and mouth, joint pain, and skin rash. After routine history and physical examination, a physician diagnosis the woman with Sjogren syndrome, a gender-biased immune disorder. The woman is treated by oral administration a pharmaceutical composition comprising a progesterone agonist as disclosed herein taken twice daily. The woman's condition is monitored and after about 3 days of treatment the woman indicates there is improvement in her health, her mouth and eyes are not as dry, the pain in her joints is reduces and her skin rash has subsided. At one and three month check-ups, the woman indicates that her mouth, eyes, and skin are normal, and she continues to have reduced joint pain. This reduction in symptoms in Sjogren syndrome indicates successful treatment with the pharmaceutical composition disclosed herein. In a similar manner, a pharmaceutical composition any of the other therapeutic compounds disclosed herein, such as, e.g., a SPRM, an estrogen antagonist, a SERM, an estrogen biosynthesis enzyme inhibitor, an androgen agonist, a SARM, or any combination thereof, may be formulated into a pharmaceutical composition and administered to the patient as described above either with or without the progesterone agonist. Furthermore, a pharmaceutical composition any of the other therapeutic compounds disclosed herein may be used to treat other female-biased immune disorders, such as, e.g., osteoarthritis, osteoporosis, chronic fatigue syndrome, fibromyalgia, irritable bowel syndrome, cataracts, interstitial cystitis, Thrombocytopenia purpura, myasthenia gravis, systemic sclerosis, or dermatomyositis.

A 31 year old female complains of enlarged thyroid, hand tremors, heat sensitivity and changed menstrual cycles. After routine history and physical examination, a physician diagnosis the woman with Graves' Disease, a gender-biased immune disorder. The woman is treated by oral administration a liquid pharmaceutical composition comprising a progesterone agonist as disclosed herein taken thrice daily. The woman's condition is monitored and after about one week of treatment the woman indicates there is improvement in her health, her thyroid is not as swollen and her hand tremors are less. At one and three month check-ups, the woman indicates that her thyroid is of normal size, she does not suffer from hand tremors, and her menstrual cycle is more regular. This reduction in symptoms in Graves' Disease indicates successful treatment with the pharmaceutical composition disclosed herein. In a similar manner, a pharmaceutical composition any of the other therapeutic compounds disclosed herein, such as, e.g., a SPRM, an estrogen antagonist, a SERM, an estrogen biosynthesis enzyme inhibitor, an androgen agonist, a SARM, or any combination thereof, may be formulated into a pharmaceutical composition and administered to the patient as described above either with or without the progesterone agonist. Furthermore, a pharmaceutical composition any of the other therapeutic compounds disclosed herein may be used to treat other female-biased immune disorders, such as, e.g., osteoarthritis, osteoporosis, chronic fatigue syndrome, fibromyalgia, irritable bowel syndrome, cataracts, interstitial cystitis, Thrombocytopenia purpura, myasthenia gravis, systemic sclerosis, or dermatomyositis.

A 39 year old female complains of breathing difficulty and chest pain. After routine history and physical examination, a physician diagnosis the woman with asthma, a gender-biased immune disorder. The woman is treated by inhalatory administration a pharmaceutical composition comprising a progesterone agonist as disclosed herein taken thrice daily. The woman's condition is monitored and after about one week of treatment the woman indicates there is improvement in her health, she can breathe more easily and her chest pains have subsided. At one and three month check-ups, the woman indicates that she does not suffer from breathing difficulty and chest pain. This reduction in symptoms in asthma indicates successful treatment with the pharmaceutical composition disclosed herein. In a similar manner, a pharmaceutical composition any of the other therapeutic compounds disclosed herein, such as, e.g., a SPRM, an estrogen antagonist, a SERM, an estrogen biosynthesis enzyme inhibitor, an androgen agonist, a SARM, or any combination thereof, may be formulated into a pharmaceutical composition and administered to the patient as described above either with or without the progesterone agonist. Furthermore, a pharmaceutical composition any of the other therapeutic compounds disclosed herein may be used to treat other female-biased immune disorders, such as, e.g., osteoarthritis, osteoporosis, chronic fatigue syndrome, fibromyalgia, irritable bowel syndrome, cataracts, interstitial cystitis, Thrombocytopenia purpura, myasthenia gravis, systemic sclerosis, or dermatomyositis.

A 46 year old female complains of nausea and vomiting, abdominal pains, and fatigue. After routine history and physical examination, a physician diagnosis the woman with autoimmune hepatitis, a gender-biased immune disorder. The woman is treated by oral administration a solid pharmaceutical composition comprising a progesterone agonist as disclosed herein taken twice daily. The woman's condition is monitored and after about 1 week of treatment the woman indicates there is improvement in her health, her nausea and vomiting have subsided, the pain in her abdominal is reduced and she is not as tired. At one and three month check-ups, the woman indicates that she has had no nausea or vomiting incidents, continues to have reduced abdominal pain and does not suffer from fatigue. This reduction in symptoms in autoimmune hepatitis indicates successful treatment with the pharmaceutical composition disclosed herein. In a similar manner, a pharmaceutical composition any of the other therapeutic compounds disclosed herein, such as, e.g., a SPRM, an estrogen antagonist, a SERM, an estrogen biosynthesis enzyme inhibitor, an androgen agonist, a SARM, or any combination thereof, may be formulated into a pharmaceutical composition and administered to the patient as described above either with or without the progesterone agonist. Furthermore, a pharmaceutical composition any of the other therapeutic compounds disclosed herein may be used to treat other female-biased immune disorders, such as, e.g., osteoarthritis, osteoporosis, chronic fatigue syndrome, fibromyalgia, irritable bowel syndrome, cataracts, interstitial cystitis, Thrombocytopenia purpura, myasthenia gravis, systemic sclerosis, or dermatomyositis.

A 49 year old female complains of joint pain, joint swelling and stiffness, and fatigue. After routine history and physical examination, a physician diagnosis the woman with Rheumatoid arthritis, a gender-biased immune disorder. The woman is treated by oral administration a pharmaceutical composition comprising a progesterone agonist as disclosed herein taken twice daily. The woman's condition is monitored and after about one week of treatment the woman indicates there is improvement in her health, the swelling and stiffness in the joints have subsided, the pain in her joints is reduce and she is not as tired. At one and three month check-ups, the woman indicates that her joints are normal and there is no pain and she continues to have energy. This reduction in symptoms in Rheumatoid arthritis indicates successful treatment with the pharmaceutical composition disclosed herein. In a similar manner, a pharmaceutical composition any of the other therapeutic compounds disclosed herein, such as, e.g., a SPRM, an estrogen antagonist, a SERM, an estrogen biosynthesis enzyme inhibitor, an androgen agonist, a SARM, or any combination thereof, may be formulated into a pharmaceutical composition and administered to the patient as described above either with or without the progesterone agonist. Furthermore, a pharmaceutical composition any of the other therapeutic compounds disclosed herein may be used to treat other female-biased immune disorders, such as, e.g., osteoarthritis, osteoporosis, chronic fatigue syndrome, fibromyalgia, irritable bowel syndrome, cataracts, interstitial cystitis, Thrombocytopenia purpura, myasthenia gravis, systemic sclerosis, or dermatomyositis.

A 51 year old female complains of hand tremors, eye pain and blurred vision, and fatigue. After routine history and physical examination, a physician diagnosis the woman with multiple sclerosis, a gender-biased immune disorder. The woman is treated by oral administration a liquid pharmaceutical composition comprising a progesterone agonist as disclosed herein taken once daily. The woman's condition is monitored and after about one week of treatment the woman indicates there is improvement in her health, her eye pain and blurred vision has subsided, her hand tremors are less and some energy has returned. At one and three month check-ups, the woman indicates that her eye pain and blurred vision is gone, she does not suffer from hand tremors, and she is not tired. This reduction in symptoms in multiple sclerosis indicates successful treatment with the pharmaceutical composition disclosed herein. In a similar manner, a pharmaceutical composition any of the other therapeutic compounds disclosed herein, such as, e.g., a SPRM, an estrogen antagonist, a SERM, an estrogen biosynthesis enzyme inhibitor, an androgen agonist, a SARM, or any combination thereof, may be formulated into a pharmaceutical composition and administered to the patient as described above either with or without the progesterone agonist. Furthermore, a pharmaceutical composition any of the other therapeutic compounds disclosed herein may be used to treat other female-biased immune disorders, such as, e.g., osteoarthritis, osteoporosis, chronic fatigue syndrome, fibromyalgia, irritable bowel syndrome, cataracts, interstitial cystitis, Thrombocytopenia purpura, myasthenia gravis, systemic sclerosis, or dermatomyositis.

A 38 year old male complains of frequent pain and stiffness in the lower back and buttocks. After routine history and physical examination, a physician diagnosis the man with ankylosing spondylitis, a gender-biased immune disorder. The man is treated by topical administration a semi-solid pharmaceutical composition comprising an estrogen agonist as disclosed herein taken thrice daily. The man's condition is monitored and after about one week of treatment the man indicates there is improvement in his health since the pain is going away. At one and three month check-ups, the man indicates that he does not suffer from having any pain. This reduction in symptoms in ankylosing spondylitis indicates successful treatment with the pharmaceutical composition disclosed herein. In a similar manner, a pharmaceutical composition any of the other therapeutic compounds disclosed herein, such as, e.g., a progesterone antagonist, a SPRM, an androgen antagonist, a SARM, a SERM, or any combination thereof, may be formulated into a pharmaceutical composition and administered to the patient as described above either with or without the estrogen agonist. Furthermore, a pharmaceutical composition any of the other therapeutic compounds disclosed herein may be used to treat other male-biased immune disorders, such as, e.g., dilated cardiomyopathy, pernicious anemia, type 1 diabetes mellitus, gastritis, Wegener's granulomatosis, idiopathic pulmonary fibrosis, Crohn's disease, or psoriasis.

A 48 year old male complains of abdominal pain, diarrhea, chills and fatigue. After routine history and physical examination, a physician diagnosis the man with primary sclerosing cholangitis, a gender-biased immune disorder. The man is treated by oral administration a solid pharmaceutical composition comprising an estrogen agonist as disclosed herein taken twice daily. The man's condition is monitored and after about one week of treatment the man indicates there is improvement in his health since the abdominal pain is reduced and the diarrhea, chills and fatigue have subsided. At one and three month check-ups, the man indicates that he does not suffer from having any abdominal pain and continues not experiencing diarrhea, chills or fatigue. This reduction in symptoms in primary sclerosing cholangitis indicates successful treatment with the pharmaceutical composition disclosed herein. In a similar manner, a pharmaceutical composition any of the other therapeutic compounds disclosed herein, such as, e.g., a progesterone antagonist, a SPRM, an androgen antagonist, a SARM, a SERM, or any combination thereof, may be formulated into a pharmaceutical composition and administered to the patient as described above either with or without the estrogen agonist. Furthermore, a pharmaceutical composition any of the other therapeutic compounds disclosed herein may be used to treat other male-biased immune disorders, such as, e.g., dilated cardiomyopathy, pernicious anemia, type 1 diabetes mellitus, gastritis, Wegener's granulomatosis, idiopathic pulmonary fibrosis, Crohn's disease, or psoriasis.

A 32 year old male complains of shortness of breath, abnormal heart beat, and light-headedness. After routine history and physical examination, a physician diagnosis the man with myocarditis, a gender-biased immune disorder. The man is treated by oral administration a liquid pharmaceutical composition comprising an estrogen agonist as disclosed herein taken twice daily. The man's condition is monitored and after about one week of treatment the man indicates there is improvement in his health since he is breathing normally and the abnormal heart beat and light-headedness have subsided. At one and three month check-ups, the man indicates that he does not suffer from shortness of breath, abnormal heart beat, or light-headedness. This reduction in symptoms in myocarditis indicates successful treatment with the pharmaceutical composition disclosed herein. In a similar manner, a pharmaceutical composition any of the other therapeutic compounds disclosed herein, such as, e.g., a progesterone antagonist, a SPRM, an androgen antagonist, a SARM, a SERM, or any combination thereof, may be formulated into a pharmaceutical composition and administered to the patient as described above either with or without the estrogen agonist. Furthermore, a pharmaceutical composition any of the other therapeutic compounds disclosed herein may be used to treat other male-biased immune disorders, such as, e.g., dilated cardiomyopathy, pernicious anemia, type 1 diabetes mellitus, gastritis, Wegener's granulomatosis, idiopathic pulmonary fibrosis, Crohn's disease, or psoriasis.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. For instance, as mass spectrometry instruments can vary slightly in determining the mass of a given analyte, the term “about” in the context of the mass of an ion or the mass/charge ratio of an ion refers to +/−0.50 atomic mass unit.

At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A method of treating a gender-biased disorder, the method comprising the step of administering a first composition comprising a progesterone agonist and the second composition comprising another therapeutic compound capable of modulating activity of a hormone for a plurality of cycles, wherein each of the plurality of cycles comprises administering the first and second compositions for a first period of time followed by a second period of time where the first and second compositions are not administered to the individual, and wherein administration of the first and second compositions reduce one or more symptoms associated with the gender-biased immune disorder.

2. The method according to claim 1, wherein the plurality of cycles is at least 2 cycles.

3. The method according to claim 1, wherein the first period of time is about 7 days to about 28 days.

4. The method according to claim 1, wherein the second period of time is about 7 days to about 28 days.

5. The method according to claim 1, wherein the gender-biased immune disorder is an autoimmune disorder.

6. The method according to claim 1, wherein the gender-biased immune disorder primarily afflicts females.

7. The method according to claim 6, wherein the gender-biased immune disorder is Hashimoto's thyroiditis, systemic lupus erythematosus (SLE), Sjogren syndrome, Graves' disease, autoimmune hepatitis, rheumatoid arthritis, osteoarthritis, osteoporosis, chronic fatigue syndrome, fibromyalgia, lupus, irritable bowel syndrome, cataracts, asthma, interstitial cystitis, Thrombocytopenia purpura, myasthenia gravis, multiple sclerosis, systemic sclerosis, or dermatomyositis

8. The method according to claim 1, wherein the gender-biased immune disorder primarily afflicts males.

9. The method according to claim 8, wherein the gender-biased immune disorder is primary sclerosing cholangitis, ankylosing spondylitis, myocarditis, dilated cardiomyopathy, Chagas disease, pernicious anemia, type 1 diabetes mellitus, gastritis, Wegener's granulomatosis, idiopathic pulmonary fibrosis, Crohn's disease, or psoriasis.

10. The method according to claim 1, wherein administration of the composition reduces the occurrence of an unwanted side.

11. The method according to claim 10, wherein the unwanted side effect includes masculinization, increased muscle mass, increased fat deposition, menorrhea, increased hair growth on face, torso, and/or extremities, feminization, mammary gland development, cancer, growth hormone effects, diabetes, mood swings, hair loss or growth on face, torso, and/or extremities, change in voice resonance or pitch, reduced sexual desire, reduced sexual arousal, reduced sexual orgasm, erectile dysfunction, impotence, infertility, or any combination thereof.

12. The method according to claim 1, wherein the first composition includes a therapeutically effective amount of the progesterone agonist.

13. The method according to claim 28, wherein the therapeutically effective amount of the therapeutic compound is in the range of about 1 mg/day to about 8,000 mg/day.

14. The method according to claim 1, wherein the second composition includes a therapeutically effective amount of the second therapeutic compound capable of modulating activity of a hormone.

15. The method according to claim 14, wherein the therapeutically effective amount of the therapeutic compound is in the range of about 1 mg/day to about 8,000 mg/day.

16. The method according to claim 1, wherein the progesterone agonist includes 11-Dehydroprogesterone, 11-Deoxycorticosterone, 17-Hydroxyprogesterone, 19-Norprogesterone, Algestone, Algestone acetonide, Algestone acetophenide, Allylestrenol, Altrenogest, Amadinone, Amadinone acetate, Anagestone, Anagestone acetate, Chlormadinone, Chlormadinone acetate, Cingestol, Cismadinone, Cismadinone acetate, Clogestone, Clogestone acetate, Clomegestone, Cyproterone, Cyproterone acetate, Delmadinone, Delmadinone acetate, Demegestone, Deoxycorticosterone, Desogestrel, Dienogest, Dimethisterone, Drospirenone, Dydrogesterone, Edogesterone, Ethisterone, Ethynerone, Ethynodiol, Ethynodiol diacetate, Etonogestrel, Etynodiol, Flugestone, Flugestone acetate, Gestaclone, Gestadienol, Gestodene, Gestonorone, Gestonorone caproate, Gestovister, Gestrinone, Gestronol, Haloprogesterone, Hydromadinone, Hydroxyprogesterone acetate, Hydroxyprogesterone caproate, Hydroxyprogesterone heptanoate, Hydroxyprogesterone hexanoate, Levonorgestrel, Lutenyl, Lynestrenol, Medrogestone, Medroxyprogesterone, Medroxyprogesterone acetate, Megestrol, Megestrol acetate, Melengestrol, Melengestrol acetate, Methylestrenolone, Methynodiol, Methynodiol diacetate, Metogest, Nandrolone, Nestorone, Nomegestrol, Nomegestrol acetate, Norelgestromin, Norethindrone, Norethindrone acetate, Norethisterone, Norethisterone acetate, Norethisterone acetate oxime, Norethisterone enanthate, Norethynodrel, Norethynodrel diacetate, Norgesterone, Norgestimate, Norgestomet, Norgestrel, Norgestrienone, Norvinisterone, Org-2058, Oxogestone, Oxogestone phenpropionate, Pentagestrone, Pregnane, Progesterone, Proligestone, Promegestone, Quingestanol, Quingestanol acetate, Quingestrone, Segesterone, Spironenone, Spironolactone, Tanaproget, Tibolone, Tigestol, Tosagestin, Trengestone, Trimegestone, ZM-182,345, or any combination thereof.

17. The method according to claim 16, wherein the progesterone agonist is Progesterone.

18. The method according to claim 1, wherein the estrogen biosynthesis enzyme inhibitor is an aromatase inhibitor, a CYP17A1 inhibitor, a 3-β-HSD inhibitor, a 17β-HSD inhibitor, or any combination thereof.

19. The method according to claim 18, wherein the aromatase inhibitor includes 1,4,6-Androstatriene-3,17-dione (ATD), 4-Androstene-3,6,17-trione (6-OXO), 4-Cyclohexylaniline, 4-Hydroxyandrostenedione, 4-Hydroxytestosterone, 5α-DHNET, Abyssinone II, Aminoglutethimide, Anastrozole, Ascorbic acid (Vitamin C), Atamestane, Bifonazole, CGP-45,688, CGS-47,645, Clotrimazole, DHT, Difeconazole, Econazole, Exemestane, Fadrozole, Fenarimol, Finrozole, Formestane, Imazalil, Isoconazole, Ketoconazole, Letrozole, Liarozole, MEN-11066, Miconazole, Minamestane, Nimorazole, NKS01, Norethindrone, ORG-33,201, Penconazole, Plomestane, Prochloraz, Propioconazole, Pyridoglutethimide, Rogletimide, Rotenone, Talarozole, Testolactone, Tioconazole, Triadimefon, Triadimenol, Troglitazone, Vorozole, YM511, Zinc, or any combination thereof.

20. The method according to claim 18, wherein the aromatase inhibitor is Letrozole.
